SEQUENCE LISTING (54 sequences)

SEQ ID NO: 1 - protein, 132 aa, Homo sapiens
MLTAAVLSCALLLALPATRGAQMGLAPMEGIRRPDQALLPELPGLGLRAPLKKTTAEQAEEDLLQEAQALAEVLDLQDREPRSSRRCVRLHESCLGQQVPCCDPCATCYCRFFNAFCYCRKLGTAMNPCSRT

SEQ ID NO: 2 - protein, 46 aa, Homo sapiens
CVRLHESCLGQQVPCCDPCATCYCRFFNAFCYCRKLGTAMNPCSRT

SEQ ID NO: 3 - protein, 33 aa, Homo sapiens
CVRLHESCLGQQVPCCDPAATCYCRFFNAFCYC

SEQ ID NO: 4 - protein, 34 aa, Artificial Sequence
  synthetic mini-AGRP
CVRLHESCLGQQVPCCDPAATCYCRFFNAFCYCR

SEQ ID NO: 5 - protein, 34 aa, Artificial Sequence; identical to SEQ ID NO: 4
  synthetic AGRP fragment

SEQ ID NO: 6 - protein, 6 aa, Artificial Sequence
  AGRP fragment
VRLHES

SEQ ID NO: 7 - protein, 6 aa, Artificial Sequence
  AGRP fragment
LGQQVP

SEQ ID NO: 8 - protein, 3 aa, Artificial Sequence
  AGRP fragment
RFF

SEQ ID NO: 9 - protein, 34 aa, Artificial Sequence
  AGRP fragment
CXXXXXXCXXXXXXCCDPXATCYCXXXNAFCYCR

SEQ ID NO: 10 - protein, 6 aa, Artificial Sequence
  AGRP fragment
XXXXXX

SEQ ID NO: 11 - protein, 6 aa, Artificial Sequence
  AGRP fragment
VXXXXX

SEQ ID NO: 12 - protein, 6 aa, Artificial Sequence
  AGRP fragment
XRXXXX

SEQ ID NO: 13 - protein, 6 aa, Artificial Sequence
  AGRP fragment
XXLXXX

SEQ ID NO: 14 - protein, 6 aa, Artificial Sequence
  AGRP fragment
XXXHXX

SEQ ID NO: 15 - protein, 6 aa, Artificial Sequence
  AGRP fragment
XXXXXS

SEQ ID NO: 16 - protein, 6 aa, Artificial Sequence
  AGRP fragment
VRXXXX

SEQ ID NO: 17 - protein, 6 aa, Artificial Sequence
  AGRP fragment
VXLXXX

SEQ ID NO: 18 - protein, 6 aa, Artificial Sequence
  AGRP fragment
VXXHXX

SEQ ID NO: 19 - protein, 6 aa, Artificial Sequence
  AGRP fragment
VXXXEX

SEQ ID NO: 20 - protein, 6 aa, Artificial Sequence
  AGRP fragment
VXXXXS

SEQ ID NO: 21 - protein, 6 aa, Artificial Sequence
  AGRP fragment
XRLXXX

SEQ ID NO: 22 - protein, 6 aa, Artificial Sequence
  AGRP fragment
XRXHXX

SEQ ID NO: 23 - protein, 6 aa, Artificial Sequence
  AGRP fragment
XRXXEX

SEQ ID NO: 24 - protein, 6 aa, Artificial Sequence
  AGRP fragment
XRXXXS

SEQ ID NO: 25 - protein, 6 aa, Artificial Sequence
  AGRP fragment
XXLHXX

SEQ ID NO: 26 - protein, 6 aa, Artificial Sequence
  AGRP fragment
XXLXXX

SEQ ID NO: 27 - protein, 6 aa, Artificial Sequence
  AGRP fragment
XXLXEX

SEQ ID NO: 28 - protein, 6 aa, Artificial Sequence
  AGRP fragment
XXLXXS

SEQ ID NO: 29 - protein, 6 aa, Artificial Sequence
  AGRP fragment
XXXHEX

SEQ ID NO: 30 - protein, 6 aa, Artificial Sequence
  AGRP fragment
XXXHXS

SEQ ID NO: 31 - protein, 6 aa, Artificial Sequence
  AGRP fragment
XXXXES

SEQ ID NO: 32 - protein, 6 aa, Artificial Sequence
  AGRP fragment
VRLXXX

SEQ ID NO: 33 - protein, 6 aa, Artificial Sequence
  AGRP fragment
VXLHXX

SEQ ID NO: 34 - protein, 6 aa, Artificial Sequence
  AGRP fragment
VRLHES

SEQ ID NO: 35 - protein, 6 aa, Artificial Sequence
  AGRP fragment
XXXXXX

SEQ ID NO: 36 - protein, 6 aa, Artificial Sequence
  AGRP fragment
LGQQVP

SEQ ID NO: 37 - protein, 6 aa, Artificial Sequence
  AGRP fragment
LXXXXX

SEQ ID NO: 38 - protein, 6 aa, Artificial Sequence
  AGRP fragment
XGXXXX

SEQ ID NO: 39 - protein, 6 aa, Artificial Sequence
  AGRP fragment
XXQXXX

SEQ ID NO: 40 - protein, 6 aa, Artificial Sequence
  AGRP fragment
XXXQXX

SEQ ID NO: 41 - protein, 6 aa, Artificial Sequence
  AGRP fragment
XXXXVX

SEQ ID NO: 42 - protein, 6 aa, Artificial Sequence
  AGRP fragment
XXXXXP

SEQ ID NO: 43 - protein, 6 aa, Artificial Sequence
  AGRP fragment
LGXXXX

SEQ ID NO: 44 - protein, 6 aa, Artificial Sequence
  AGRP fragment
LXQXXX

SEQ ID NO: 45 - protein, 6 aa, Artificial Sequence
  AGRP fragment
LXXQXX

SEQ ID NO: 46 - protein, 6 aa, Artificial Sequence
  AGRP fragment
LXXXVX

SEQ ID NO: 47 - protein, 6 aa, Artificial Sequence
  AGRP fragment
LXXXXP

SEQ ID NO: 48 - protein, 6 aa, Artificial Sequence
  AGRP fragment
LGQXXX

SEQ ID NO: 49 - protein, 3 aa, Artificial Sequence
  AGRP fragment
RXX

SEQ ID NO: 50 - protein, 3 aa, Artificial Sequence
  AGRP fragment
XFX

SEQ ID NO: 51 - protein, 3 aa, Artificial Sequence
  AGRP fragment
RFX

SEQ ID NO: 52 - protein, 3 aa, Artificial Sequence
  AGRP fragment
RXF

SEQ ID NO: 53 - protein, 3 aa, Artificial Sequence
  AGRP fragment
XFF

SEQ ID NO: 54 - protein, 6 aa, Artificial Sequence
  AGRP fragment
RFFNAF

(19) United States
(12) Patent Application Publication
(10) Pub. No.: US 2003/0064921 A1
(43) Pub. Date: Apr. 3, 2003

(54) Methods and compounds for modulating melanocortin receptor ligand binding and activity

(75) Inventors: Glenn L. Millhauser, Santa Cruz, CA (US); Darren Thompson, Santa Cruz, CA (US); Kimberly Bolin, Santa Cruz, CA (US); D. Joe Anderson, Ames, IA (US); Joseph C. McNulty, Santa Cruz, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 09/851,586

(22) Filed: May 8, 2001

Related U.S. Application Data
(63) Continuation-in-part of application No. PCT/US99/25201, filed on Oct. 27, 1999.
(60) Provisional application No. 60/203,071, filed on May 9, 2000.
(60) Provisional application No. 60/226,047, filed on Aug. 16, 2000.

(30) Foreign Application Priority Data
Oct. 27, 1999 (WO) PCT/US99/25201

(51) Int. Cl. A61K 38/17; A61K 38/08

(52) U.S. Cl. 514/12; 514/17

(57) ABSTRACT

This invention relates to methods and agonist/antagonist compounds for modulating melanocortin receptor-ligand binding. Also included is a method of identifying agonists and/or antagonists that bind to a ligand binding site for a melanocortin receptor of interest. Agonists and antagonists of ligand binding to melanocortin receptors also are provided. The invention is exemplified by identification and manipulation of the C-terminus of the human agouti related protein, which binds melanocortin receptors 3 and 4, and the production of AGRP peptidomimetics that are melanocortin receptor ligands. The methods can be applied to other melanocortin receptor agonists and antagonists.

### CROSS-REFERENCE TO RELATED APPLICATIONS

This application is a continuation-in-part of PCT application no: PCT/US99125201, filed on Oct. 27, 1999, designating the United States of America, and also claims benefit of and priority to U.S. provisional application No: 601203,071, filed on May 9, 2000, and U.S. provisional application No: 60/226,047, filed on Aug. 16, 2000, all of which are incorporated herein by reference, in their entirety, for all purposes.

### STATEMENT AS TO RIGHTS TO INVENTIONS MADE UNDER FEDERALLY SPONSORED RESEARCH AND DEVELOPMENT

This invention was made with Government support under Grant Nos. GM46870, and DK58606 awarded by the National Institutes of Health. The Government of the United States of America may have certain rights in this invention.

### FIELD OF THE INVENTION

The present invention relates to methods and compounds for modulating melanocortin receptor-ligand binding.

### BACKGROUND OF THE INVENTION

The melanocortin (MC) receptors are a group of cell surface proteins that mediate a variety of physiological effects, including: regulation of adrenal gland function, such as production of glucocorticoid, cortisol and aldosterone; control of melanocyte growth and pigment production; hemoregulation; immunomodulation; and analgesia. Five distinct MC receptors have been cloned and are expressed in a variety of tissues, including melanocytes, adrenal cortex, brain, gut, placenta, skeletal muscle, lung, spleen, thymus, bone marrow, pituitary, gonads and adipose tissue. Two MC eceptors, MC3R and MC4R, are expressed in brain tissue, while MC1r is found in the skin. A variety of ligands termed melanocortins function as agonists that stimulate the activity of MC receptors. The melanocortins include melanocyte-stimulating hormones (MSH) such as alph-MSH, beta-MSH and gamma-MSH, as well as adrenocorticotropic hormone. Another MCR ligand that has been discovered is the Agouti Related Protein (AGRP), which is an antagonist.

AGRP is a natural antagonist of melanocortin receptors 3 and 4 (MC3R and MC4R). Recent biochemical investigations have identified AGRP as playing a major role in the regulation of mammalian feeding behavior. The human AGRP is a 132 residue polypeptide (SEQ ID NO:1) that is a naturally occurring competitive antagonist of melanocortin receptors 3 and 4 (“MC3r” and “MC4r”), the overexpression of which results in adult onset obesity and diabetes in mice (Shutter et al. (1997) Genes Dev. 11:593-602; Huszar et al. (1997) Cell 88:131-141; Hahn, et al. (1997) Nature Neurosci. 1:271-171).

AGRP binding to MC4r in particular is the subject of intense interest since knockout mice that do not express MC4r exhibit the same phenotype as caused by overexpression of AGRP (Huszar, et al., supra). There is also evidence for the parallel expression of AGRP and neuropeptide Y in the arcuate nucleus of the hypothalamus, with neuropeptide Y known to stimulate feeding (Hahn, et al., supra). This region of the brain also expresses MC4r and is involved in energy homeostasis. Research has also focused on other melanocortin receptors, their antagonists and methods for modulating receptor activity. See for example, Wei et al., WO9943709.

The growing body of evidence linking AGRP to weight control has yet to elucidate its exact mechanism of action. However, studies on AGRP do benefit from analogy to the much more widely studied agouti protein, as AGRP was originally identified through the homology of its C-terminal region with the same region of the agouti protein (Shutter et al., supra). The agouti protein has been a focal point in obesity research for a number of years, since ectopic expression of the wild-type protein in mice leads to obesity and related disorders, i.e. the same symptoms as the overexpression of the more recently identified AGRP (Klebig, et al., (1995) Proc. Natl. Acad. Sci. USA 92:4728-4732; Michaud, et al. (1997) J. Endocrinol 155:207-209. However, unlike AGRP, agouti has distinct expression patterns in mice and humans, making in vivo work with mice less applicable to human obesity disorders. AGRP, like agouti, is selective for MC3r and MC4r but has approximately 100-fold greater binding affinity than agouti at these receptors (Fong, et al. (1997) Biochem. Res. Commun. 237:629 611).

While full length agouti and AGRP are only 25% homologous, in their 46 residue Cys-rich C-terminal regions, nine of the 10 Cys residues are spatially conserved and there are a further 10 identical residues giving ˜40% sequence identity. Three consecutive, conserved residues RFF (111-113 in human AGRP) were determined to be essential to the biological activity of both agouti (Kiefer, et al. (1997) Biochemistry 36:2084-2090; Kiefer, et al. (1998) Biochemistry 37: 991-997) and AGRP (Tota et al. (1999) Biochemistry 38:897-904). Two recent investigations have shown that the chemically synthesized C-terminal region of AGRP competitively antagonizes α-melanocyte stimulating hormone (α-MSH) at melanocortin receptors with equal or greater potency than the full proteins (Quillan, et al. (1998) FEBS Lett. 428:59-62; Yang, et al. (1999) Mol. Edocrinol. 13:148-155), consistent with similar findings for agouti (Willard, et al. (1995) Biochemistry 34:12341-12346). Thus the Cys-rich C-terminal region of AGRP, is referred to as minimized agouti related protein (“MARP”, CVRLH₅ESCLG₁₀QQVPC₁₅CDPCA₂₀TCYCR₂₅FFNA F₃₀CYCR K₃₅LGTAM₄₀NPCSR₄₅T, SEQ ID NO:2, where subscripts denote amino acid position).

The covalent structure of MARP exhibits five disulfide bonds, which exist between the following ten Cys residues (Bures, et al. (1988) Biochemistry 37:12172-12177): Cys_{1 and CyS}₁₆; Cys₈ and Cys₂₂; Cys₁₅ and Cys₃₃; Cys₁₉ and Cys₄₃; Cys₂₄ and Cys₃₁.

Despite the important biological activities of AGRP, no experimental 3D structure has been available for this protein. The inhibitor cystine knot (“ICK”) family of proteins are also disulfide-rich and the structures of these invertebrate toxins have been used to suggest possible structures for the agouti and AGRP C-terminal regions (Kiefer, et al (1988) Biochemistry 37:991-997; Tota, et al., supra). Indeed, the recently reported disulfide map for AGRP and a construct containing the C-terminal domain demonstrate ICK-like pairings (Norton, et al. (1998) Toxicon 36:1573-1583) of the 10 Cys residues: 1-16, 8-22, 15-33, 19-43, 24-31 (using MARP numbering, Bures, et al., supra). Beyond such modeling, it is believed that the only structural data published on either agouti or AGRP are circular dichroism (CD) spectra which suggest that both proteins have little regular secondary structure, although there may be some β-sheet structure, consistent with ICK structural characteristics (Willard, et al., supra; Rosenfeld, et al. (1998) Biochemistry 37:16041-16052).

U.S. patents of interest in this area include: U.S. Pat. Nos. 5,994,087; 5,932,779; 5,869,257; 5,843,652; 5,817,787; 5,766,877; 5,731,408; 5,703,220; and 5,622,860. Also of interest are: WO 99/64002; WO 99157148; WO 99/55679; WO 99/55832; WO 99/54358; WO 99/50295; WO 99/43709; WO 99/31508; WO 99/21571; WO 98/56914; WO 98/10068; and WO 97/43412.

### SUMMARY OF THE INVENTION

This invention provides a detailed description of the three-dimensional (3D) structure (NMR structure) in solution of the human AGRP Cys-rich C-terminal region. In addition, further minimization of human AGRP is achieved and a minimal sequence (designated herein as MARP-33) is identified that provides both activity and receptor specificity against MC3r and MC4r. Information about this region permits design of compounds that bind to the ligand binding site of melanocortin receptors and modulate ligand binding to the receptor. The compounds include agonists and antagonists that modulate melanocortin receptor activity by promoting (agonists) or blocking (antagonists) ligand binding to the receptor and/or by activating the receptor themselves. In particular, using information provided herein, a class of peptidomimetics (e.g. non-peptide ligands) are provided that are ligands for the melanocortin receptor and that can block and/or modulate activity of the melanocortin receptors.

In one preferred embodiment, the peptides this embodiments provides a melanocortin receptor ligand that is a polypeptide comprising a peptide sequence having the formula: CX¹X²X³X⁴X⁵X⁶X⁷X⁸X⁹X¹⁰X¹¹X¹²CCDPX ¹³ ATCYCX¹⁴X¹⁵X¹⁶NAFCYCRₙ (Formula I), where X¹, X², X³, X⁴, X⁵, X⁶, X⁷, X⁸, X⁹, X¹⁰, X¹¹, X¹², X¹³, X¹⁴, X¹⁵, and X¹⁶ are independently selected amino acids, and n is zero or one. The amino acids include native amino acids, both D- and L-form amino acids, and modified or derivatized amino acids or amino acid analogues. In certain particularly preferred embodiments, X¹, X², X³, X⁴, X⁵, X⁶, X⁷, X⁸, X⁹, X¹⁰, X¹¹, X¹², X¹³, X¹⁴, X¹⁵, and X¹⁶ are independently selected from the group consisting of alanine, asparagine, arginine,aspartic acid, cysteine, glutamine, glutamic acid, glycine, histidine, isoleucine, leucine, lysine, methionine, phenylalanine, proline, serine, threonine, tryptophan, tyrosine, and valine. In certain embodiments, the polypeptide consists of a polypeptide having the formula shown above (Formula I).

In certain preferred embodiments, the peptide is not AGRP and said polypeptide is not MARP. In certain preferred embodiments, the polypeptide excludes one or more of the final 13 residues of MARP (residues 34-46 of MARP). In certain embodiments, the polypeptide is not CVRLHESCLGQQVPCCDPAATCYCRFFNAFCYC (SEQ ID NO:3) or does not comprise this sequence.

In certain embodiments, X¹X²X³X⁴X⁵X⁶ is VRLHES, or conservative substitutions thereof, and/or X⁷X⁸X⁹X¹⁰X¹¹X¹² is LGQQVP, or conservative substitutions thereof, and/or X¹⁴X¹⁵X¹⁶ is RFF or conservative substitutions thereof. In certain embodiments, X¹³ is not a cysteine and in particularly preferred embodiments, X¹³ is A.

In addition to the peptide melanocortin ligands described above, this invention provides a non-peptide melanocortin receptor ligand of the structural formula of Formula I shown herein in which B, U₁, U₂, R, R₁ and R₂ are independently selected from the group consisting of: hydrogen, alkyl, derivatized alkyl, cycloalkyl, derivatized cycloalkyl, halocycloalkyl, aloxycycloalkyl, heterocycloalkyl, aryl, arylalkyl, heteroaryl, and heteroarylalkyl; J is selected from the group consisting of carbon, nitrogen, silicon, and sulfur; X is selected from the group consisting of hydrogen, carbon, nitrogen, oxygen, silicon, and sulfur; and Z is selected from the group consisting of a continuing peptide bond, a hydroxyl; —NH₂—, —NH-(n), and —N-(n,n′), and —O-(y), where where n and n′ are independently selected from the group consisting of alkyl, alkenyl, alkynyl, cycloalkyl, aryl, arylalkyl, heteroaryl, heteroarylalkyl, or a derivatized form thereof, and y is selected from the group consisting of alkyl, alkenyl, alkynyl, cycloalkyl, aryl, arylalkyl, heteroaryl heteroarylalkyl, or a derivatized form thereof. In preferred embodiments, the ligand is a ligand for a MC3r and/or an MC4r melanocortin receptor. Particularly preferred non-peptide ligands have a molecular weight ranging from about 200 to 1000 daltons. In preferred embodiments, the ligand has a structure that mimics the backbone of the AGRP active loop. Preferred ligands comprise a terminal gaunidino moiety and/or at least one methylbenzyl moiety. Particularly preferred ligands include the ligands of Formulas III, IV, V, and VI, shown herein.

In another embodiment, this invention also provides libraries for screening for modulators of a melanocortin receptor. Preferred libraries comprise a plurality of polypeptide and/or non-polypeptide members that are ligands (e.g. as described above) that bind to a melanocortin receptor (e.g. MC3r, MC4r, etc.). Preferred libraries comprise at least 10, more preferably at least 100, and most preferably at least 1000 different members. In certain embodiments, the libraries are provided in multi-well plates.

Also provided are pharmaceutical compositions comprising one or more of the melanocortin-binding peptide and/or non-peptide ligands of this invention (e.g. as described above). The pharmaceutical compositions optionally further comprise a pharmaceutically acceptable excipient.

In certain embodiments, this invention provides methods of modulating the activity of a melanocortin receptor (e.g. MC3r, MC4r, etc.) and/or methods of modulating a melanocortin receptor mediated physiological process. The methods involve contacting the receptor with a peptide ligand and/or with a non-peptide ligand of this invention (e.g. as described above). In preferred embodiments, the contacting blockage of the receptor site and/or upregulation or downregulation of melanocortin receptor activity (e.g. as measured by cAMP assays). In certain embodiments the ligand is a melanocortin agonist. In certain embodiments, the ligand is a melanocortin receptor antagonist.

This invention also provides methods of prescreening for a modulator of a melanocortin receptor (e.g. MC3r, MC4r, etc.). The methods involve contacting a melanocortin receptor with one or more of the melanocortin receptor peptide ligands and/or non-peptide ligands of this invention (e.g. the ligands described above), and detecting binding of the peptide to the melanocortin receptor wherein specific binding of the peptide to the melanocortin receptor indicates that said peptide is a potential modulator of the melanocortin receptor.

In still another embodiment, this invention provides methods of screening for a modulator of melanocortin receptor activity. The methods involve contacting a melanocortin receptor (e.g. MC3r, MC4r, etc.) with a peptide ligand or a non-peptide ligand of this invention and detecting activity of the melanocortin receptor wherein a difference in activity of the receptor, as compared to a control, indicates that the ligand is a modulator of melanocortin receptor activity. In preferred embodiments, the control is a negative control comprising the same assay without the ligand.

This invention also provides a method of identifying a compound that modulates ligand binding to a melanocortin receptor, where the method involves modeling test compounds that fit spatially into a melanocortin receptor ligand binding site of interest using an atomic structural model of a melanocortin receptor binding region or portion thereof; screening the test compounds in an assay characterized by binding of a test compound to a melanocortin receptor ligand binding site; and identifying a test compound that modulates ligand binding to said melanocortin receptor. In preferred embodiments, the melanocortin receptor binding region comprises the minimized agouti related protein receptor binding region (mini-AGRP) or a portion thereof. In preferred embodiments, the atomic structural model comprises atomic coordinates of amino acid residues corresponding to residues 1-18 of the N-terminal loop of the minimized agouti related protein (residues 1-18 of SEQ ID NO:2), residues 19-13 of the central loop of the minimized agouti related protein (residues 19-34 of SEQ ID NO:2), and residues 35-46 of the C-terminal loop of the minimized agouti related protein (residues 35-46 of SEQ ID NO:2). In certain preferred embodiments, the atomic structural model comprises atomic coordinates of amino acid residues corresponding to residues 19-34 of the central loop of the minimized agouti related protein (residues 19-34 of SEQ ID NO:2) and at least residues 15-18 of the N-terminal loop of the minimized agouti related protein (residues 15-18 of SEQ ID NO:2). In certain preferred embodiments, the atomic structural model comprises atomic coordinates of amino acid residues corresponding to residues 19-34 of the central loop of the minimized agouti related protein (residues 19-34 of SEQ ID NO:2) and at least 20% of the contiguous or non-contiguous residues or their atoms are selected from residues 1-18 of the N-terminal loop of the minimized agouti related protein (residues 1-18 of SEQ ID NO:2). The atomic structural model can comprises atomic coordinates of amino acid residues corresponding to residues 24-31 of the active loop of the minimized agouti related protein (residues 24-31 of SEQ ID NO:2). In certain embodiments, the atomic structural model comprises atomic coordinates of amino acid residues corresponding to residues 25-27 of the active loop of the minimized agouti related protein (residues 25-27 of SEQ ID NO:2). The screening is preferably in vitro. In certain embodiments, the screening is high throughput screening (HTS). Preferred assays include, but are not limited to biological assays. The test compound can be provided from a library of compounds and preferred test compounds are agonists or antagonists of ligand binding. Particularly preferred test compounds include a small organic molecule, a peptide, or peptidomimetic.

In another embodiment this invention provides methods for identifying an agonist or antagonist of ligand binding to a melanocortin receptor, said method comprising the steps of: providing the atomic coordinates of a melanocortin receptor binding region or portion thereof to a computerized modeling system; modeling compounds which match or mimic the receptor binding region and thus fit spatially into the melanocortin receptor ligand binding site; and identifying in an assay for melanocortin receptor activity a compound that increases or decreases the activity of said melanocortin receptor by binding the ligand binding site of said melanocortin receptor, whereby an agonist or antagonist of ligand binding is identified. In preferred embodiments, the melanocortin receptor binding region comprises the minimized agouti related protein receptor binding region or portion thereof.

Also provided is a machine-readable data storage medium, comprising a data storage material encoded with machine readable data which, when using a machine programmed with instructions for using said data, is capable of displaying a graphical three-dimensional representation of a molecule that binds a melanocortin receptor comprising structure coordinates of amino acid residues corresponding to residues 1-18 of the N-terminal loop of the minimized agouti related protein (residues 1-18 of SEQ ID NO:2), residues 19-13 of the central loop of the minimized agouti related protein (residues 19-34 of SEQ ID NO:2), and residues 35-46 of the C-terminal loop of the minimized agouti related protein (residues 35-46 of SEQ ID NO:2), or a homologue of said molecule. In certain embodiments, the molecule is a melanocortin receptor agonist or antagonist. In particularly preferred embodiments, the molecule is defined by the set of structure coordinates depicted in Table 4 or Table 5, or a homologue of the molecule, the homologue having a root mean square deviation from the backbone atoms of said amino acids of not more than 10, preferably no more than 5 and most preferably no more than 2.54 Å.

A machine-readable data storage medium is also provided that comprises a data storage material encoded with a first set of machine readable data that, when combined with a second set of machine readable data, using a machine programmed with instructions for using said first set of data and said second set of data, can determine at least a portion of the structure coordinates corresponding to the second set of machine readable data, wherein the first set of data comprises a Fourier transform of at least a portion of the structural coordinates selected from the group consisting of coordinates depicted in Table 4 or Table 5; and the second set of data comprises an X-ray diffraction pattern of a molecule.

In still another embodiment this invention provides an NMR structure of a minimized agouti related protein (mini-AGRP), embodied in a computer readable media.

This invention also provides a polypeptide comprising the amino acid sequence: CVRLHESCLGQQVPCCDPAATCYCRFFNAFCYC (SEQ ID NO:3) or a modified form thereof, where the polypeptide is not a full-length AGRP and the polypeptide is not a MARP. Preferred such polypeptides are chemically synthesized.

Also provided is a method of treating a disease state in mammals that is alleviated by treatment with a polypeptide having an amino acid sequence: CVRLHESCLGQQVP CCDPAATCYCRFFNAFCYC (SEQ ID NO:3) where the method comprises administering to a mammal in need of such a treatment a therapeutically effective amount of said polypeptide, or a pharmaceutically acceptable salt thereof. In certain embodiments, the disease state is a wasting syndrome. This invention also includes a pharmaceutical composition comprising a therapeutically effective amount of a polypeptide of the sequence: CVRLHESCLGQQVPC CDPAATCYCRFFNAFCYC (SEQ ID NO:3) or a pharmaceutically acceptable salt thereof.

### DEFINITIONS

The terms “residue” or “amino acid” as used herein refers to natural, synthetic, or modified amino acids (amino acid analogues). Such amino acids include both “D” and “L” forms. Various amino acid analogues include, but are not limited to, 2-aminoadipic acid, 3-aminoadipic acid, beta-alanine, beta-aminopropionic acid, 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4- diaminobutyric acid, desmosine, 2,2′-diaminopimelic acid, 2,3-diaminopropionic acid, N-ethylglycine, N-ethylasparagine, hydroxylysine, allo-hydroxylysine, 3-hydroxyproline, 4-hydroxyproline, isodesmosine, allo-isoleucine, N-methylglycine, sarcosine, N-methylisoleucine, 6-N-methyllysine, norvaline, norleucine, ornithine, etc.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The term also includes variants on the traditional peptide linkage joining the amino acids making up the polypeptide.

The term “peptidomimetic” as used herein refers to a molecule that structurally and chemically resembles a peptide of two or more amino acids with respect to the features critical for a particular desired activity (e.g. receptor specificity, activity, etc.). The term “peptidomimetic” is includes peptide analogs that serve as appropriate substitutes for peptides in interactions with e.g., receptors and enzymes. Peptidomimetics can be “peptides” incorporating modified residues and/or backbones, e.g. that may have improved pharmacokinetic properties as a result of proteolytic stability, or unique structural and/or hydrogen bonding motifs, etc. Peptidomimetics also include organic molecules that are capable of mimicing one more properties (e.g. binding specificity, affinity, etc.) of a peptide. Such organic molecule typically comprise a “scaffold” that mimics part or all of the 3-dimensional structure of the subject peptide and places appropriate functional groups in a spatial orientation sufficient to achieve the desired functional properties. Particularly preferred peptidomimetics include small organic molecules.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term database refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

The term “conservative substitution” is used in reference to proteins, peptides, etc. to reflect amino acid substitutions that do not substantially alter the activity (specificity or binding affinity) of the molecule. Typically, conservative amino acid substitutions involve substitution of one amino acid for another amino acid with similar chemical properties (e.g. charge or hydrophobicity). The following six groups each contain amino acids that are typical conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

The terms “binding partner”, or “capture agent”, or a member of a “binding pair” refers to molecules that specifically bind other molecules to form a binding complex such as antibody-antigen, lectin-carbohydrate, nucleic acid-nucleic acid, biotin-avidin, etc.

The term “specifically binds”, as used herein, when referring to a biomolecule (e.g., protein, nucleic acid, antibody, receptor, etc.), refers to a binding reaction which is determinative of the presence biomolecule in heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl, pentyl, and the like.

“Alkenyl” means a linear monovalent hydrocarbon radical of two to six carbon atoms or a branched monovalent hydrocarbon radical of three to six carbon atoms containing at least one double bond, e.g., ethenyl, 2-propenyl, and the like.

“Alkynyl” means a linear monovalent hydrocarbon radical of two to six carbon atoms or a branched monovalent hydrocarbon radical of three to six carbon atoms containing at least one triple bond, e.g., ethynyl, propynyl, butynyl, and the like.

“Cycloalkyl” means a cyclic saturated monovalent hydrocarbon radical of three to seven carbon atoms, e.g., cyclopropyl, cyclohexyl, and the like.

“Halo” means fluoro, chloro, bromo, and iodo.

“Haloalkyl” means alkyl substituted with one or more halogen atoms, including those substituted with different halogens, e.g., —CH₂Cl, —CF₃, —CH₂CF₃, —CF₂CF₃, —CH₂CCl₃, and the like.

“Alkoxy”, “alkenyloxy”, “cycloalkyloxy”, or “haloalkyloxy” means a radical —OR where R is alkyl, alkenyl, cycloalkyl, or haloalkyl respectively as defined above, e.g., methoxy, ethoxy, propoxy, 2-propoxy, ethenyloxy, cyclopropyloxy, cyclobutyloxy, —OCH₂ Cl, —OCF₃, and the like.

“Alkylthio” or “cycloalkylthio” means a radical —SR where R is alkyl or cycloalkyl respectively as defined above, e.g., methylthio, butylthio, cyclopropylthio, and the like.

“Acyl” means a radical —C(O)R where R is hydrogen, alkyl, or haloalkyl as defined above, e.g., formyl, acetyl, trifluoroacetyl, butanoyl, and the like.

“Amino” means a radical —NH₂

“Monosubstituted amino” means a radical —NHR where R is alkyl or acyl, e.g., methylamino, (1-methylethyl)amino, and the like.

“Disubstituted amino” means a radical —NRR′ where R and R′ are independently alkyl or acyl, e.g.,dimethylamino, methylethylamino, di(1-methylethyl)amino, and the like.

“Hydroxyalkyl” means a linear monovalent hydrocarbon radical of two to six carbon atoms or a branched monovalent hydrocarbon radical of three to six carbons substituted with one or two hydroxy groups, provided that if two hydroxy groups are present they are not both on the same carbon atom. Representative examples include, but are not limited to, 2-hydroxyethyl, 2-hydroxypropyl, 3-hydroxypropyl, 1-(hydroxymethyl)-2-methylpropyl, 2-hydroxybutyl, 3-hydroxybutyl, 4hydroxybutyl, 2,3-dihydroxypropyl, 1-(hydroxymethyl)-2-hydroxyethyl, 2,3-dihydroxybutyl, 3,4dihydroxybutyl and 2-(hydroxymethyl)-3-hydroxypropyl, 2-hydroxyethyl, 2,3-dihydroxypropyl, and 1-(hydroxymethyl)-2-hydroxyethyl.

“Alkoxyalkyl” means a linear monovalent hydrocarbon radical of one to six carbon atoms or a branched monovalent hydrocarbon radical of three to six carbons substituted with at least one alkoxy group as defined above, e.g., 2-methoxyethyl, 2-methoxypropyl, and the like.

“Hydroxyalkyloxy” or “alkoxyalkyloxy” means a radical-OR where R is hydroxyalkyl or alkoxyalkyl respectively as defined above, e.g., 2-hydroxyethyloxy, 2-methoxyethyloxy, and the like.

“Aminoalkyl” means a linear monovalent hydrocarbon radical of two to six carbon atoms or a branched monovalent hydrocarbon radical of three to six carbons substituted with at least one —NRR′ where R and R′ are independently selected from hydrogen, alkyl, or acyl, e.g., 2-aminoethyl, 2-N,N-diethylaminopropyl, 2-N-acetylaminoethyl, and the like.

“Aryl” means a monovalent monocyclic or bicyclic aromatic hydrocarbon radical of 6 to 12 ring atoms, and optionally substituted independently with one or more substituents selected from alkyl, haloalkyl, cycloalkyl, alkoxy, alkylthio, halo, nitro, acyl, cyano, amino, monosubstituted amino, disubstituted amino, hydroxy, carboxy, or alkoxycarbonyl. Representative examples include, but are not limited to, phenyl, biphenyl, 1-naphthyl, and 2-naphthyl and the derivatives thereof.

“Heteroaryl” means a monovalent monocyclic or bicyclic aromatic radical of 5 to 10 ring atoms containing one or more, sometimes one or two ring heteroatoms selected from N, O, or S, the remaining ring atoms being C. The heteroaryl ring is optionally substituted independently with one or more substituents, sometimes one or two substituents, selected from alkyl, haloalkyl, cycloalkyl, alkoxy, alkylthio, halo, nitro, acyl, cyano, amino, monosubstituted amino, disubstituted amino, hydroxy, carboxy, or alkoxycarbonyl. Specifically the term heteroaryl includes, but is not limited to, pyridyl, pyrrolyl, thienyl, furanyl, indolyl, quinolyl, benzopyranyl, and thiazolyl, and the derivatives thereof.

“Heterocycloamino” means a saturated monovalent cyclic group of 3 to 8 ring atoms, wherein at least one ring atom is N and optionally contains a second ring heteroatom selected from the group consisting of N, O, or S(O)ₙ (where n is an integer from 0 to 2), the remaining ring atoms being C. The heterocycloamino ring may be optionally fused to a benzene ring or it may be optionally substituted independently with one or more substituents, sometimes one or two substituents, selected from alkyl, haloalkyl, cycloalkyl, cycloalkylalkyl, aryl, aralkyl, heteroaryl, heteroaralkyl, halo, cyano, acyl, amino, monosubstituted amino, disubstituted amino, carboxy, or alkoxycarbonyl. More specifically the term heterocycloamino includes, but is not limited to, pyrrolidino, piperidino, morpholino, piperazino, indolino, and thiomorpholino, and the derivatives thereof.

“Heterocyclo” means a saturated monovalent cyclic group of 3 to 8 ring atoms in which one or two ring atoms are heteroatoms selected from N, O, or S(O)ₙ, where n is an integer from 0 to 2, the remaining ring atoms being C. The heterocyclo ring may be optionally fused to a benzene ring or it may be optionally substituted independently with one or more substituents, sometimes one or two substituents, selected from alkyl, haloalkyl, cycloalkyl, cycloalkylalkyl, aryl, aralkyl, heteroaralkyl, halo, cyano, acyl, monosubstituted amino, disubstituted amino, carboxy, or alkoxycarbonyl. More specifically the term heterocyclo includes, but is not limited to, pyrrolidino, piperidino, morpholino, piperazino, tetrahydropyranyl, and thiomorpholino, and the derivatives thereof.

“Cycloalkylalkyl” means a radical —R^{a}R^{b} where R^{a} is an alkylene group and R^{b} is a cycloalkyl group as defined above e.g., cyclopropylmethyl, cyclohexylpropyl, 3-cyclohexyl-2-methylpropyl, and the like.

“Cycloalkylalkyloxy” means a radical —OR where R is a cycloalkylalkyl group as defined above e.g., cyclopropylmethyloxy, 3-cyclohexylpropyloxy, and the like.

“Aralkyl” means a radical —R^{a}R^{b} where R^{a} is an alkylene group and R^{b} is an aryl group as defined above e.g., benzyl, phenylethyl, 3-(3-chlorophenyl)-2-methylpentyl, and the like.

“Heteroaralkyl” means a radical —R^{a}R^{b} where R^{a} is an alkylene group and R^{b} is a heteroaryl group as defined above e.g., 2-,3-, or 4-pyridylmethyl, furan-2-ylmethyl and the like.

“Heterocycloalkyl” means a radical —R^{a}R^{b} where R^{a} is an alkylene group and R^{b} is a heterocyclo group as defined above e.g., morpholin-4-ylethyl, tetrahydrofuran-2-ylmethyl and the like.

A “pro-drug” is a compound that releases an active drug (e.g. a mini-AGRP, a peptidomimetic as described herein) when such prodrug is administered to a mammalian subject. In certain embodiments, prodrugs are prepared by modifying functional groups present in the peptidomimetics of this invention or by modifying mini-AGRP polypeptides in such a way that the modifications may be cleaved in vivo to release the active compound. Peptidomimetic prodrugs include the peptideomimetics described herein wherein a hydroxy, amino, or sulfhydryl group in compound is bonded to any group that may be cleaved in vivo to regenerate the free hydroxyl, amino, or sulfhydryl group, respectively. Examples of prodrugs include, but are not limited to esters (e.g., acetate, formate, and benzoate derivatives), carbamates (e.g., N,N-dimethylaminocarbonyl) of hydroxy functional groups in compounds of formula (I), and the like.

“Optional” or “optionally” means that the subsequently described event or circumstance may, but need not, occur, and that the description includes instances where the event or circumstance occurs and instances in which it does not. For example, “heterocyclo group optionally mono- or di- substituted with an alkyl group” means that the alkyl may, but need not, be present, and the description includes situations where the heterocyclo group is mono- or disubstituted with an alkyl group and situations where the heterocyclo group is not substituted with the alkyl group.

Compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The compounds of this invention may possess one or more asymmetric centers; such compounds can therefore be produced as individual (R)- or (S)- stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 1992).

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. “A pharmaceutically acceptable excipient” as used in the specification and claims includes both one and more than one such excipient.

A “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4methylbicyclo>2.2.2!oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Treating” or “treatment” of a disease includes: (1) preventing the disease, i.e. causing the clinical symptoms of the disease not to develop in a mammal that may be exposed to or predisposed to the disease but does not yet experience or display symptoms of the disease, (2) inhibiting the disease, i.e., arresting or reducing the development of the disease or its clinical symptoms, or (3) relieving the disease, i.e., causing regression of the disease or its clinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The term database refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like

### BRIEF DESCRIPTION OF THE DRAWINGS

FIGS. 1A and 1B show the Cα backbone of the MARP minized average structure determined at 500 mHz and 800 mHz, respectively. FIG. 1A: The N-terminal loop, central loop and C-terminal loop are indicated. Disulfide bonds are represented by dashed lines. Spheres represent residues with amides protected from HX for more than 12 hours, more than 24 hours and more than 8 days. FIG. 1B shows a C(alpha) backbone representation of the lowest energy AGRP(87-132) structure of the family of 40 determined from 800 MHz NOESY data and deposited in the PDB (code: 1HYK). The N-terminal loop is show in light grey at the lower left,the central loop is dark grey (top middle), and the C-terminal loop is shown at the lower right. The disulfide bonds are included as thin dashed white lines. The spheres highlight residues the amides of which were protected from HX: medium gray>12 hours, light gray>24 hours and dark gray>8 days.

FIG. 2A depicts the heavy atom (non-hydrogen) backbone representation of MARP for 14 NMR structures with residues 1-34 fit to the minimized average structure (Root Mean Square Deviation, “RMSD” 1.49 Å). Only the minimized average structure (thick cylinder) is shown for the more disordered C-terminal loop. FIG. 2B shows an all atom backbone representation of AGRP(87-132) for the 40 structures deposited in the PDB calculated from 800 MHz NOESY data. Only the backbone atoms of 1-34 have been aligned (RMSD=0.34 angstroms). The disulfide-bound residues are indicated with straight lines interconnecting regions of the backbone, and are included for all members of the family.

FIG. 3 shows the backbone atoms for residues 24-31 of the family of 20 structures with residues 24-31 fit to the minimized average structure. The side chain heavy atoms of residues 25, 26 and 27, essential for activity, are shown.

FIG. 4 illustrates a reaction scheme for the synthesis of peptidomimetics of this invention.

### DETAILED DESCRIPTION

The agouti related protein (“AGRP”) is a mammalian signaling molecule, involved in weight homeostasis, that causes adult onset obesity when overexpressed in mice. AGRP was originally identified by homology to the agouti protein, another potent signaling molecule involved in obesity disorders in mice. While AGRP's exact mechanism of action is unknown, it has been identified as a competitive antagonist of melanocortin receptors 3 and 4 (“MC3r” and “MC4r”). MC4r in particular is implicated in the hypothalamic control of feeding behavior. Full length agouti and AGRP are only 25% homologous, however, their active C-terminal regions are ˜40% homologous, with nine out of the 10 Cys residues spatially conserved. Until now, 3D structures have not been available for either agouti, AGRP or their C-terminal regions.

In one embodiment, this invention provides a detailed three-dimensional (3D) NMR structure in solution of the human AGRP Cys-rich C-terminal region as determined by ¹H NMR using a protein prepared by total chemical synthesis. As used herein the term “the NMR structure” is understood to refer to the minimized average of the family of NMR structures. Because biochemical investigations demonstrate that this minimal region retains full biological activity, this protein is referred to herein as minimized agouti related protein (“MARP”). MARP residues 1-46 (SEQ ID NO:2), correspond to human AGRP residues 87-132 (residues 87-132 of SEQ ID NO:1). Thus, human AGRP numbering is obtained by adding 86 to MARP numbering.

MARP's topology is characterized by three large loops (referred to herein as the N-terminal loop, the central loop and the C-terminal loop), with four of the five disulfide bridges at the base of the structure, and an absence of canonical secondary structure such as helices or sheets. Two of the three loops are structurally well characterized by the NMR data as indicated by low RMSDs. The region of MARP containing the RFF triplet (Tota, et al., supra) (residues 25-27 in MARP) necessary for function is located in one of the best defined regions of the protein. While previously reported structural models of the C-terminal region of AGRP were attempted based on Cys homology between AGRP and certain toxin proteins, Cys spacing was not sufficient to correctly determine the 3D fold of the molecule. It was initially thought, based on 500 mHz data that MARP did not adopt an ICK-like fold, however, new data obtained at 800 mHZ (Table 5 herein) shows that MARP does adopt the ICK-like fold.

The 3D structure of MARP provided herein presents a basis for the development of methods and compositions for identifying compounds that modulate melanocortin receptor activity, in particular the activity of MC3r and MC4r. The 3D structure presents precise structural information that permits the rational design of compounds that preferentially modulate MC4r or MC3r activity.

Based on an analysis of this structure, a minimal AGRP domain is identified (designated as a mini-AGRP) that shows both melanocortin activity and melanocortin specificity. Residues are identified that can be systematically or randomly altered to produce a large number of mini-AGRPs having differing receptor specificity and/or binding affinity.

Collections of such mini-AGRPs provide convenient libraries that can be screened to identify mini-AGRPs having particular activity/specificity profiles. THese AGRPs can be used to modulate melanocortin receptor activity (in vivo or in vitro) or they can be used as “lead compounds” for the design of peptido mimetics.

In addition a class of peptidomimetics is identified herein that are specific ligands to melanocortin receptors (e.g. MC3r and/or MC4r). In certain embodiments, the peptidomimetics are used to modulate melanocortin receptor activity in vivo or in vitro. In other embodiments, they are provided as libraries that can be screened for peptidomimetics having particular desired specificity/activity profiles.

I. Structure of MARP

The 3D structure of MARP is characterized by three loops held together at the base by an apparent scaffold of four disulfide bonds 1-16, 8-22, 15-33 and 1943. The fifth disulfide bond, 24-31, further stabilizes the base of the active loop which presents the RFF triplet on the protein surface. There is no identifiable canonical helical structure. The 800 mHz data reveal a small beta sheet structure. The RFF triplet is critical for the activity of MARP as a competitive antagonist of α-MSH stimulated activation of MC4r signaling. The structure described herein shows that MARP is structured to present the side chains of the RFF triplet on the surface of the protein and to the surrounding solvent. Recent work demonstrates that MARP is much more active than smaller AGRP derived peptides containing the RFF triplet (Tota, et al. (1999) Biochemistry 38:897-904). Thus, the detailed fold of the central loop and perhaps the presence of the N- and C-terminal loops are important for AGRP function. In addition, based upon work with chimeras of melanocortin receptors, we believe the N- and C-terminal loops confer receptor subtype specificity.

The previous absence of structural data on both AGRP and agouti encouraged the modeling of the C-terminal regions of these proteins onto the ICK family (Norton, et al., supra)) which is characterized by homologous Cys spacing (Kiefer, et al., Biochemistry 37:991-997 (1998); Tota, et al., supra). The ICK family of proteins primarily consists of small (<60 residues) disulfide-rich (three or four disulfides) toxin proteins from the venom of spiders and cone snails, which function as ion channel antagonists (Norton, et al., supra). The coincidence between the function of the majority of these toxins and the recent description of part of the agouti protein's mechanism of action being calcium dependent (Kim, et al., FASEB J. 10: 1646-1652 (1996); Kim, et al., Am. J. Physiol. 272:E379-384 (1997); Jones, et al., Am. J. Physiol. 270:E192-196 (1996)) further encouraged these homology modeling efforts. The ICK motif in particular is characterized by the topology of the three disulfide bonds corresponding to 1-16, 8-22 and 15-33 in MARP. In the ICK motif the first two disulfide bonds with their intervening main chain atoms form a topological circle through which the third disulfide bond passes, forming the cystine knot (Norton, et al., supra). The motif is further characterized by the identification of an irregular triple stranded antiparallel β-sheet, roughly corresponding to residues 6-8, 20-24 and 31-34 in MARP. The remaining two disulfide bonds in MARP each occur in individual ICK proteins as separate examples of potential “non-motif” disulfide bonds, although no examples of ICK motif proteins with five disulfide bonds have been observed.

Despite these apparent similarities, the experimental structure of MARP determined at 500 mHz, suggested that this protein did not satisfy the criteria required for inclusion in the ICK family. While the first two disulfide bonds in MARP 1-16 and 8-22, together with the polypeptide backbone form a topological circle, none of the remaining disulfides passes through the circle to form a cystine knot. Instead, disulfide bond 15-33 is positioned adjacent to the circle with all of the fold on one side of this circle. In addition, MARP lacks the β-sheet found in ICK family proteins. The experimental determination of the distinctive 3D structure of MARP described herein suggests that Cys spacing and even the disulfide map of small Cys-rich proteins may not always be sufficient to accurately predict protein folds. These results speak to the potential limitations of “homology modeling” of protein structures, and may have important implications for the emerging field of genomic structural biology. However, based on the 800 mHz NMR data, we have not assigned MARP to the ICK class.

As described in the Examples, ligand binding studies, and analysis of atomic models derived from the MARP NMR structure reveal for the first time a previously unknown structure for MARP and its receptor binding region. By “receptor binding region” is intended a structural segment or segments of melanocortin receptor ligands, and MARP in particular, folded in such a way so as to give the proper geometry and amino acid residue conformation for binding to a melanocortin receptor. By “ligand binding site” is intended a structural segment or segments of melanocortin receptor polypeptide chain folded in such a way so as to give the proper geometry and amino acid residue conformation for binding a ligand. These are the physical arrangement of protein atoms in three-dimensional space forming a receptor binding region or a ligand binding site pocket or cavity.

The MARP structure has three major loops: the N-terminal loop, residues 1-18 (residues 1-18 of SEQ ID NO:2), the central loop, residues 19-34 (residues 19-34 of SEQ ID NO:2) and the C-terminal loop, residues 35-46 (residues 35-46 of SEQ ID NO:2). Residues forming the receptor binding region are amino acids corresponding to (i.e., the same as or equivalent to) residues 24-31 of the central loop (residues 24-31 of SEQ ID NO:2), referred to herein as the “active” loop. In particular, residues 25, 26 and 27 (the “RFF” triplet) of the active loop are critical for activity.

It has been found that a polypeptide comprising the central loop, preferably at least a portion of the N-terminal loop, is desirable for optimal biological activity. As used herein, the term “at least a portion of the N-terminal loop” is intended to mean a sequence that corresponds to (i.e., the same as or equivalent to), at least residues 15 to 18 of the N-terminal loop (residues 15-18 of SEQ ID NO:2), preferably at least residues 8 to 18 of the N-terminal loop (residues 8-18 of SEQ ID NO:2), and is also intended to include all of the N-terminal loop, i.e., residues 1 to 18 of the N-terminal loop (residues 1-18 of SEQ ID NO:2). The term is also intended to mean a sequence that corresponds to at least 20%, preferably at least 60%, and more preferably at least 90%, of the contiguous or non-contiguous amino acid residues or their atoms selected from amino acid residues 1 to 18 of the N-terminal loop (residues 1-18 of SEQ ID NO:2).

II. Structure of Mini-AGRP

Another aspect of the invention also pertains to the identification of “minimal” polypeptides (mini-AGRPs) that show specific binding and/or activity at melanocortin receptors show. Certain preferred mini-AGRPs are strong antagonists of melanocortin receptor types 3 and 4, and are useful for antagonizing melanocortin receptor in a variety of contexts, e.g. for the treatment of eating disorders and obesity. As indicated above, research has demonstrated that the final 46 residues of the human AGRP sequence (“MARP”), possesses full receptor antagonist activity. The structure of MARP, solved by use of nuclear magnetic resonance and described herein, indicated that the final 13 residues of MARP are unstructured are unlikely to play a significant role in receptor binding or antagonism. Thus, substantial further minimization of human AGRP was achieved. Specifically, the sequence (indicated by the single letter amino acid code): CVRLH₅ESCLG₁₀QQVPC₁₅CDPAA₂₀TCYCR₂₅FFNAF₃₀CYC (SEQ ID NO:3) designated “MARP-33” (based upon its 33 amino acid length) is believed to fold with proper native-like disulfide bonds and possesses the full biological activity of MARP. This sequence is equivalent to the first 33 amino acids of MARP but contain a Cys to Ala substitution at position 19 (indicated as “A”) to avoid having a non-bridged thiol.

Another mini-AGRP was made having the sequence: CVRLHESCLGQQVPCCDPAATCYCRFFNAFCYCR (SEQ ID NO:4). This sequence is equivalent to the first 34 amino acids of MARP, but contain a Cys to Ala substitution at position 19 (indicated as “A”) to avoid having a non-bridged thiol. The additional terminal residue (R) facilitates solid state synthesis. This mini-AGRP was shown to specifically bind an antagonize melanocortin receptors.

In addition, the mini-ARGPs can be modified to improve binding specificity, and/or to alter activity (e.g. agonist, antagonist, competitive inhibitor, etc.). Such modifications include, but are not limited to changing the sequence to increase binding affinity, to increase the level of antagonism and to increase stability of the molecule. In particularly preferred embodiments, the amino acid substitutions are made in the RFF residues (residues 25-27 of SEQ ID NO:3) and/or in the N-terminal loop (e.g. one or more of residues 2-7 and/or residues 9-14 of SEQ ID NO:3).

In particularly preferred embodiments, mini-ARGPs are represented by formula I:

CX¹X²X³X⁴X⁵X⁶X⁷X⁸X⁹X¹⁰X¹¹X¹²CCDPX¹³ATCYCX¹⁴X¹⁵X¹⁶NAFCYCRₙ I

where X¹, X², X³, X⁴, X⁵, X⁶, X⁷, X⁸, X⁹, X¹⁰, X¹¹, X¹², X¹³, X¹⁴, X¹⁵, and X¹⁶ are independently selected amino acids (including natural, synthetic, or modified amino acids); and n is zero or one. In certain embodiments, in each of the varied domains, one or more of the native residues can be preserved. Thus, for example, X¹X²X³X⁴X⁵X⁶ includes, but is not limited to VX²X³X⁴X⁵X⁶, X¹RX³X⁴X⁵X⁶, X¹X²LX⁴X⁵X⁶, X¹X²X³HX⁵X⁶, X¹X²X³X⁴⁵S, VRX³X⁴X⁵X⁶, VX²LX⁴X⁵X⁶, VX²X³HX⁵X⁶, VX²X³X⁴EX⁶, VX²X³X⁴X⁵S, X¹RLX⁴X⁵X⁶, X¹RX³HX⁵X⁶, X¹RX³X⁴EX⁶, X¹RX³X⁴X⁵S, X¹X²LHX⁵X⁶, X¹X²LX⁴X⁵X⁶, X¹X²LX⁴EX⁶, X¹X²LX⁴X⁵S, X¹X²X³HEX⁶, X¹X²X³HX⁵S, X¹X²X³X⁴ES, VRLX⁴X⁵X⁶, VX²LHX⁵X⁶, VRLHES and the like. Similar permutations are available for X⁷X⁸X⁹X¹⁰X¹¹X¹² (e.g. LGQQVP, LX⁸X⁹X¹⁰X¹¹X¹², X⁷GX⁹X¹⁰X¹¹X¹², X⁷X⁸QX¹⁰X¹¹X¹², X⁷X⁸X⁹QX¹¹X¹², X⁷X⁸X⁹X¹⁰VX¹², X⁷X⁸X⁹X¹⁰X¹¹P, LGX⁹X¹⁰X¹¹X¹², LX⁸QX¹⁰X¹¹X¹², LX⁸X⁹QX¹¹X¹², LX⁸X⁹X¹⁰VX¹², LX⁸X⁹X¹⁰X¹¹P, LGQX¹⁰X¹¹X¹², and the like). Similarly, the “RFF” domain can be fully mutated or can retain one or more of the native residues. Thus, for example, X¹⁴X¹⁵X¹⁶ includes RFF, R¹⁵X¹⁵X¹⁶, X¹⁴FX¹⁶, X¹⁴X¹⁵F, RFX¹⁶, RX¹⁵F, X¹⁵FF. In certain preferred embodiments, X¹³ is not cysteine.

The mini-AGRPs form a class of melanocortin receptor binding and/or modulating agents and thus find utility as modulators (e.g. upregulators, downregulators, competitive inhibitors) of melanocortin receptors, particular MC3r and/or MC4r. The compounds can be used individually or combined into a library suitable for screening for members having particular activities, binding affinities, and the like.

Certain mini-AGRPs are good potential therapeutics for the treatment of eating disorders and obesity, or can be used as therapeutic lead compounds for the development of therapeutics (e.g. as models for peptidomimetics, etc.). Where the compounds are used as therapetics, they can be administered to a patient (human or non-human mammal) in need thereof, to increase eating and fat deposition.

III. Preparation of Mini-AGRP's and Mini-AGRP Libraries.

In certain embodiments, this invention provides libraries of mini-AGRPs. The libraries typically comprise a plurality of

Preferred libraries comprise at least 20, preferably at least 50, more preferably at least 100, and most preferably at least 10,000, 50,000, 100,000, or even at least 1,000,000 different members.

Using the mini-AGRP sequence information provided herein, such libraries can be routinely prepared using methods well known to those of skill in the art. Such methods include, but are not limited to “traditional” chemical syntheses methods, light-directed chemical syntheses, and recombinant expression.

Solid phase peptide synthesis in which the C-terminal amino acid of the sequence is attached to an insoluble support followed by sequential addition of the remaining amino acids in the sequence is the preferred method for preparing the peptide compounds of the present invention. Techniques for solid phase synthesis are well known t those of skill in the art (see, e.g., Barany and Merrifield, Solid-Phase Peptide Synthesis; pp. 3-284 in The Peptides: Analysis, Synthesis, Biology. Vol. 2: Special Methods in Peptide Synthesis, Part A., Merrifield, et al. J. Am. Chem. Soc. 85, 2149-2156 (1963), and Gross and Meienhofer, eds. Academic press, N.Y., 1980 and Stewart et al., Solid Phase Peptide Synthesis, 2nd ed. Pierce Chem. Co., Rockford, Ill. (1984)). Solid phase synthesis is most easily accomplished with commercially available peptide synthesizers utilizing FMOC or TBOC chemistry.

In particularly preferred embodiments, peptide synthesis is performed using Fmoc synthesis. For example, AGRP C-terminal portion (MARP) can be readily synthesized using techniques described in Yang, et al. supra. Each desired peptide can be individually synthesized. Where large numbers of different peptides are desired (e.g. for a library) combinatorial synthesis methods are available. Combinatorial peptide synthesis can be performed according to the methods of Furka et al., (1991) Int. J. Pept. Protein Res. 37 487-493. At the desired positions that are to be altered the synthesis resins are separated coupled with the desired amino acid(s) and then pooled for the next coupling reaction. Such methods are easily accomplished using multiple peptide synthesizers.

After synthesis, the proteins are purified if necessary (e.g. via HPCL), re-folded and disulfide bonds are formed. Formation of intrapeptide disulfide bonds is often achieved by oxidation of the free thiol or sulfur-protected precursors under varying reaction conditions (see, e.g., Andreu et al. (1994) Pages 91-169 In Peptide Synthesis Protocols; Pennington, M. W., Bunn, B. M., Ed.; Humana Press: New Jersey; Moroder et al. (1996) Biopolymers 40: 207-234; Annis and Barany (1997) Meth. Enzymol., 289: 198-221; Tam et al. (1991) J. Am. Chem. Soc. 111:, 6657-6662; Munson and Barany (1993) J. Am. Chem. Soc. 115: 10203-10216; Shik (1993) J. Org. Chem. 58: 3003-3008; Annis and Barany (1998) J. Am. Chem. Soc., 120: 7226-7238; Shi and Rabenstein (1999) J. Org. Chem. 64: 4590-4595). Suitable oxidants include, but are not limited to iodine, thallium(III) trifluroacetate (Fujii et al.(1987) Chem. Pharm. Bull., 35: 2339-2347), and the like.

Alternatively, recombinant expression methods can be used to produce the peptides of this invention. In this approach, a nucleic acid encoding the desired polypeptide (and optionally a purification tag, e.g. HiS6) is provided in an appropriate vector. A cell (e.g. E. coli, SF-3 cell, etc.) is transfected with the nucleic acid and, under appropriate conditions, transcribes and translates the desired protein. The protein is then recovered, re-folded, if necessary and cross-linked using standard methods well known to those of skill in the art.

Molecular cloning techniques to achieve these ends are known in the art. A wide variety of cloning and in vitro amplification methods are suitable for the construction of recombinant nucleic acids. Examples of these techniques and instructions sufficient to direct persons of skill through many cloning exercises are found, e.g., in Berger and Kimmel, (1989) Guide to Molecular Cloning Techniques, Methods in Enzymology volume 152 Academic Press, Inc., San Diego, Calif.; Sambrook et al. (1989) Molecular Cloning—A Laboratory Manual (2nd ed.) Vol. 1-3, Cold Spring Harbor Laboratory, Cold Spring Harbor Press, NY; and Ausubel et al. (1994) Current Protocols in Molecular Biology, Current Protocols, a joint venture between Greene Publishing Associates, Inc. and John Wiley & Sons, Inc.,

IV. Use of Molecular Models to Design Small Molecules that Bind the Melanocortin Receptor Ligand Binding Site.

One aspect of the invention involves methods for identifying and designing small molecules that bind to the ligand binding site using atomic models of MARP. In particular, the invention provides methods of identifying a compound that modulates ligand binding to a melanocortin receptor. The method involves modeling test compounds that mimic or match the 3D conformation of MARP and therefore are expected to fit spatially into a melanocortin receptor ligand binding site of interest, using an atomic structural model of a melanocortin receptor binding region or portion thereof, preferably comprising the MARP receptor binding region or portion thereof. The test compounds can fit spatially into the ligand binding site of interest based upon a geometric fit of its three-dimensional structure or based upon the spatial arrangement of atoms presenting specific chemical properties such as charge and hydrophobicity. The test compounds are then screened in an assay, such as a biological assay, characterized by binding of a test compound to a melanocortin receptor ligand binding site, and identifying a test compound that modulates ligand binding to the melanocortin receptor. Details of the atomic structural model are described in detail herein.

The atomic coordinates of MARP and its receptor binding region, provided herein, can be used for modeling to identify other compounds or fragments that bind melanocortin receptors. By “modeling” is intended quantitative and qualitative analysis of molecular structure/function based on atomic structural information and receptor-ligand agonists/antagonists interaction models. This includes conventional numeric-based molecular dynamic and energy minimization models, interactive computer graphic models, modified molecular mechanics models, distance geometry and other structure-based constraint models. Modeling is preferably performed using a computer and may be further optimized using known methods. By “fits spatially” is intended that the three-dimensional structure of a compound is accommodated geometrically by a cavity or pocket of a melanocortin receptor ligand binding site or by a pattern of charge or hydrophobicity.

Compounds of particular interest fit spatially and preferentially into the ligand binding site. By “fits spatially and preferentially” is intended that a compound possesses a three-dimensional structure and conformation for selectively interacting with a melanocortin receptor ligand binding site. Compounds that fit spatially and preferentially into the ligand binding site interact with amino acid residues forming the ligand binding site. More specifically, these compounds mimic or match the MARP receptor binding region. The present invention also includes a method for identifying a compound capable of selectively modulating ligand binding to different melanocortin receptors. The method comprises the steps of modeling test compounds that fit spatially and preferentially into the ligand binding site of a melanocortin receptor of interest using an atomic structural model of a MARP, screening the test compounds in a biological assay for melanocortin receptor activity characterized by preferential binding of a test compound to the ligand binding site of a melanocortin receptor, and identifying a test compound that selectively modulates the activity of a melanocortin receptor. Such receptor-specific compounds are selected that exploit differences between the ligand binding sites of one type of receptor versus a second type of receptor.

The invention also is applicable to generating new compounds that distinguish melanocortin receptor isoforms. This can facilitate generation of either tissue-specific or function-specific compounds.

The receptor-specific compounds of the invention preferably interact with conformationally constrained residues of the ligand binding site that are conserved among one type of receptor compared to a second type of receptor. “Conformationally constrained” is intended to refer to the three-dimensional structure of a chemical or moiety thereof having certain rotations about its bonds fixed by various local geometric and physical-chemical constraints. Conformationally constrained structural features of a ligand binding site include residues that have their natural flexible conformations fixed by various geometric and physical-chemical constraints, such as local backbone, local side chain, and topological constraints. These types of constraints are exploited to restrict positioning of atoms involved in receptor-ligand recognition and binding.

As described in the Examples, residues 25, 26 and 27 of the MARP receptor binding region are shown to be critical for activity. These three residues, along with the overall structure and composition of the N-terminus and central loop, appear to be necessary for optimal biological activity. Accordingly, modification to these residues can be exploited in the identification and design of compounds that modulate ligand binding to one melanocortin receptor compared to another.

For modeling, docking algorithms and computer programs that employ them can be used to identify compounds that match or mimic the MARP receptor binding region. For example, docking programs can be used to predict how a small molecule of interest can interact with the melanocortin receptor ligand binding site. Fragment-based docking also can be used in building molecules de novo inside the ligand binding site, by placing chemical fragments that complement the site to optimize intermolecular interactions. The techniques can be used to optimize the geometry of the binding interactions. This design approach has been made possible by identification of the receptor binding region structure thus, the principles of molecular recognition can now be used to design a compound which matches the structure of this region. Compounds that structurally match or mimic the MARP receptor binding region serve as a starting point for an iterative design, synthesis and test cycle in which new compounds are selected and optimized for desired properties including affinity, efficacy, and selectivity. For example, the compounds can be subjected to addition modification, such as replacement and/or addition of R-group substituents of a core structure identified for a particular class of binding compounds, modeling and/or activity screening if desired, and then subjected to additional rounds of testing.

Computationally small molecule databases can be screened for chemical entities or compounds that can bind in whole, or in part, to a melanocortin receptor ligand binding site of interest. In this screening, the quality of fit of such entities or compounds to the binding site may be judged either by shape complementarity (DesJalais et al., J. Med. Chem. (1988) 31:722-729) or by estimated interaction energy (Meng et al., J. Comp. Chem. (1992) 13:505-524). The molecule databases include any virtual or physical database, such as electronic and physical compound library databases, and are preferably used in developing compounds that modulate ligand binding.

Compounds can be designed intelligently by exploiting available structural and functional information by gaining an understanding of the quantitative structure-activity relationship (QSAR), using that understanding to design new compound libraries, particularly focused libraries having chemical diversity of one or more particular groups of a core structure, and incorporating any structural data into that iterative design process. For example, one skilled in the art may use one of several methods to screen chemical entities or fragments to compare them to the 3D structure of the AGRP C-terminus and thus, their ability to associate with the ligand binding site of a melanocortin receptor of interest. This process may begin by visual inspection of, for example, the receptor binding region on the computer screen. Selected fragments or chemical entities may then be positioned into all or part of the region. Docking may be accomplished using software such as Quanta and Sybyl, followed by energy minimization and molecular dynamics with standard molecular mechanics force-fields, such as CHARMM and AMBER.

Residues comprising a receptor binding region can be modeled to look for energetic contributions and interaction with the bound chemical entity. For example, a compound or fragment can be designed to contain hydrophobic groups that interact with hydrophobic residues of the ligand binding site.

Specialized computer programs may also assist in the process of selecting chemical entity fragments or whole compounds. These include: GRID (Goodford, J. Med. Chem. (1985) 28:849-857; available from Oxford University, Oxford, UK); MCSS (Miranker et al., Proteins: Structure, Function and Genetics, (1991) 11:29-34; available from Molecular Simulations, Burlington, Mass.); AUTODOCK (Goodsell et al., Proteins: Structure, Function and Genetics (1990) 8:195-202; available from Scripps Research Institute, La Jolla, Calif.); and DOCK (Kuntz et al, J. Mol. Biol. (1982) 161:269-288; available from University of California, San Francisco, Calif.).

Additional commercially available computer databases for small molecular compounds include Cambridge Structural Database and Fine Chemical Database (Rusinko, Chem. Des. Auto. News (1993) 8:44-47).

Once suitable chemical entities or fragments have been selected, they can be assembled into a single compound. Assembly may be proceeded by visual inspection of the relationship of the fragments to each other on the three-dimensional image displayed on a computer screen in relation to the structure coordinate s of a melanocortin receptor. This can be followed by manual model building using software such as Quanta or Sybyl.

Useful programs to aid one of skill in the art in connecting the individual chemical entities or fragments include: CAVEAT (Bartlett et al., “CAVEAT: A Program to Facilitate the Structure-Derived Design of Biologically Active Molecules”, In: Molecular Recognition in Chemical and Biological Problems”, Special Pub., Royal Chem. Soc. (1989) 78:182-196; CAVEAT is available from the University of California, Berkeley, Calif.); 3D Database systems such as MACCS-3D (MDL Information Systems, San Leandro, Calif.; reviewed in Martin, J. Med. Chem. (1992) 35:2145-2154); and HOOK (available from Molecular Simulations, Burlington, Mass.).

In addition to building a compound in a step-wise fashion, one fragment or chemical entity at a time as described above, compounds that bind to a ligand binding site of interest also may be designed as a whole or de novo using some portion(s) of the AGRP C-terminus, which is a molecule known to bind to the site. These methods include: LUDI (Bohm, J. Comp. Aid. Molec. Design (1992) 6:61-78; LUDI is available from Biosym Technologies, San Diego, Calif.); LEGEND (Nishibata et al., Tetrahedron (1991) 47:8985; LEGEND is available from Molecular Simulations, Burlington, Mass.); and LeapFrog (available from Tripos Associates, St. Louis, Mo.).

Other molecular modeling techniques may also be employed in accordance with this invention. See, for example, Cohen et al., J. Med. Chem. (1990) 33:883-894); Navia et al., Curr. Opin. Struct. Biol. (1992) 2:202-210). For example, where the structures of test compounds are known, a model of the test compound may be superimposed over the model of the structure of the invention. Numerous methods and techniques are known in the art for performing this step, any of which may be used. See, for example, Farmer, “Drug Design,” Ariens, E. J., ed., 10:119-143 (Academic Press, New York, 1980); U.S. Pat. Nos. 5,331,573; 5,500,807; Verlinde, Structure, (1994) 2:577-587); and Kuntz et al., Science, (1992) 257:1078-1082). The model building techniques and computer evaluation systems described herein are not a limitation on the present invention.

Using these computer modeling systems a large number of compounds may be quickly and easily examined and expensive and lengthy biochemical testing avoided. Moreover, the need for actual synthesis of many compounds can be substantially reduced and/or effectively eliminated.

V. AGRP Peptidomimetics.

Using the three-dimensional model of AGRP provided herein, a class of non-peptide melanocortin receptor ligands (peptidomimetics) were designed. The subject ligands are low molecular weight compounds that structurally mimic the AGRP active loop backbone. The subject non-peptide compounds find use in a variety of different applications, including the modulation of melanocortin receptor mediated physiological processes.

By ligands of melanocortin receptors (MCR) is meant that the subject non-peptide compounds bind to melanocortin receptors. In many embodiments, the subject compounds preferentially bind to the following melanocortin receptors: MC3R, as described in U.S. Pat. No. 5,837,521, the disclosure of which is herein incorporated by reference; and MC4R, as described in U.S. Pat. No. 5,703,220, the disclosure of which is herein incorporated by reference. As the subject non-peptide compounds are MCR ligands, the subject compounds bind to an MCR with an affinity corresponding to a K_{d} of about 50 mM or lower, preferably of about 5 mM or lower, more preferably of about 500 μM or lower, and most preferably about 50 μM, 5 μM, or 1 μM or lower. In many embodiments, the affinity of the subject non-peptide compounds for an MCR, usually either MC3r or MC4r, ranges from about ranges from about 100 μM to 0.1 pM, and more usually about 10 μM to 0.1 nM As the subject compounds are non-peptide compounds, they are protease resistant. By protease resistant is meant that the subject compounds are resistant to proteolytic cleavage, at least by the proteases described in Miller et al. (1994), Bioorg Med Chem Let 4: 2657-2662. The subject non-peptide compounds are low molecular weight compounds. By low molecular weight is meant that the subject compounds have a molecular weight that ranges from about 200 to 1100 g/mol, usually from about 300 to 900 g/mol and more usually from about 400 to 800 g/mol.

A feature of the subject non-peptide compounds is that they structurally mimic the active loop 3-D conformation when bound by the receptor. By active loop is meant residues 111-116 or Arg-Phe-Phe-Asn-Ala-Phe (SEQ ID NO:______) of the Agouti Related Protein. More specifically, the subject non-peptide compounds are characterized by substantially structurally mimicking the backbone phi angle of amino acid 113 in AGRP, i.e. Phe113 phi=−55.4°, and the U₁-U₂ interatomic distance (see structure below). As the subject compounds substantially structurally mimic the active loop, in 9 of 10 lowest energy structures calculated with distance geometry the following requirements should be met.

The phi angle mimicking amino acid 113 of AGRP, any deviation from the above angles should not exceed about −90° to −10° or 10° to 90°, usually about −85° to −20° or 20° to 85°, and more usually about −80° to −30° or 30° to 80° in phi space. The interatomic U₁-U₂ distance should not exceed about 6.5 Å, usually about 5.7 Å, and more usually about about 5.5 Å.

In many preferred embodiments, the subject non-peptide ligands have the formula of Formula II:

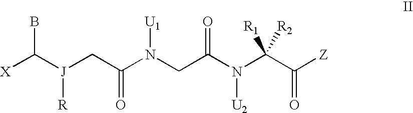

where: each B, U₁, U₂, R, R₁ and R₂ is independently selected from the group consisting of: hydrogen; alkyl; derivatized alkyl, e.g. halo alkyl, alkoxyalkyl, heteroalkyl, etc.; cycloalkyl; derivatized cycloalkyl, e.g. halocycloalkyl, aloxycycloalkyl, heterocycloalkyl; aryl; arylalkyl; heteroaryl; or heteroarylalkyl; J is carbon, nitrogen, silicon, or sulfur; X is hydrogen, carbon, nitrogen, oxygen, silicon, or sulfur; Z is a continuing peptide bond; hydroxyl; amide of the form —NH₂—, —NH-(n) or —N-(n,n′), where n or n′ can be any alkyl, alkenyl, alkynyl, cycloalkyl, aryl, arylalkyl, heteroaryl or heteroarylalkyl, or derivatized form thereof; or ester of the form —O-(y) where y can be any alkyl, alkenyl, alkynyl, cycloalkyl, aryl, arylalkyl, heteroaryl or heteroarylalkyl, or derivatized form thereof. In certain preferred embodiments, B comprises a heteroalkyl moiety, where in certain preferred embodiments the heteroalkyl moiety comprises a guanidino moiety. In certain preferred embodiments, each U is a cycloalkyl, preferably arylalkyl, and more preferably methylbenzyl. In many preferred embodiments, the subject compounds are compounds in which B, R, R₁ and R₂ are as described above.

Specific non-peptide compounds of interest include the compounds of Formulas III, IV, V, and VI, shown below:

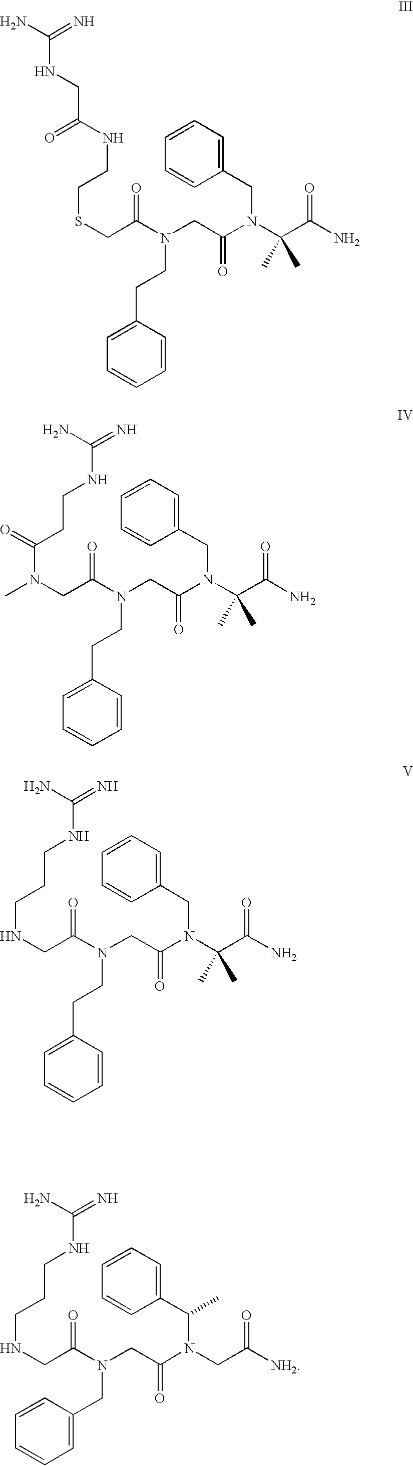

The subject compounds may be synthesized using any convenient protocol. In one representative protocol, the peptoid portion of the subject molecules is synthesized using a submonomer approach, where a representative submonomer synthesis protocol that may be adapted to synthesize the subject compounds is described in U.S. Pat. Nos. 5,977,301 and 5,831,005; the disclosures of which are herein incorporated by reference. Following production of the peptoid portion, the terminal “B” group as described above is added to the terminus of the peptoid portion of the compound. A representative scheme for preparation of the subject compounds is provided in the Experimental Section, infra.

VII. Uses of Melanocortin Receptor Ligands.

A) Use as Melanocortin Receptor Probes.

The melanocortin receptor ligands of this invention are useful in a wide variety of contexts. Because the ligands specifically bind the melanocortin receptor, they can be used as probes to specifically detect/localize melanocortin receptors (in vivo, in vitro, in cell culture, etc.). Thus, the ligands can be used to detect the presence or absence or to quantify the expression level of a melanocortin receptor. In such applications, the receptor ligands are preferably labeled with a detectable label.

Detectable labels suitable for use in the present invention include any composition detectable by spectroscopic, photochemical, biochemical, immunochemical, electrical, optical or chemical means. Useful labels in the present invention include, but are not limited to biotin for staining with labeled streptavidin conjugate, magnetic beads (e.g., Dynabeads™), fluorescent dyes (e.g., fluorescein, texas red, rhodamine), fluorescent proteins (e.g., green fluorescent protein (GFP), red fluorescent protein (RFP), and the like), radiolabels (e.g., ³H, ¹²⁵I, ³⁵S, ¹⁴C, or ³²P), enzymes (e.g., horse radish peroxidase, alkaline phosphatase and others commonly used in an ELISA), chemiluminescent labels (e.g. luciferins), and colorimetric labels such as colloidal gold (e.g., gold particles in the 40-80 nm diameter size range scatter green light with high efficiency) or colored glass or plastic (e.g., polystyrene, polypropylene, latex, etc.) beads. Other suitable labels include spin labels, e.g., provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe-CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018). Patents teaching the use of labels include U.S. Pat. Nos. 3,817,837; 3,850,752; 3,939,350; 3,996,345; 4,277,437; 4,275,149; and 4,366,241.

The label may be added to the melanocortin receptor ligand prior to, or after the ligand is contacted to the sample. So called “direct labels” are detectable labels that are directly attached to or incorporated into the ligand prior application of the ligand to the sample. In contrast, so called “indirect labels” are joined to the bound ligand after it has contacted the sample. Often, the indirect label becomes attached to a binding moiety present on the ligand before it is contacted to the sample. Thus, for example, the ligand may be biotinylated before it is used in the assay. After the ligand is bound to melanocortin receptor(s) in the sample, an avidin-conjugated fluorophore will bind the biotin on the ligand thereby providing a label that is easily detected.

The label is attached to the ligand directly or through a linker moiety. In certain embodiments, the label can be conveniently attached to an amino or carboxyl terminus of the ligand or to the R group of any amino acid(s) comprising the ligand as long as it does not interfere with specific binding of the ligand.

The label is detected using a method appropriate to the nature of the label. Thus, for example, enzymatic labels are detected by providing the appropriate substrate and reaction conditions for the enzyme and detecting loss of substrate or increase of reaction product. Radioactive labels are detected, e.g. via scintillography. Fluorescent labels and/or colorimetric labels are detected using optical methods (e.g. fluorometry, image analysis, etc.).

B) Use in Altering Melanocortin Receptor Activity.

In certain preferred embodiments, the melanocortin receptor ligands of this invention are used to alter (modulate) melanocortin receptor activity. The ligand can act as a simple competitive inhibitor blocking access to the receptor by its native ligand. Alternatively the ligands of this invention can act as receptor agonists or antagonists.

The melanocortin receptor can be modulated in vivo, or in vitro. Of particular interest is use of such compounds in a method of modulating melanocortin receptor activity in a mammal by administering to a mammal in need thereof a sufficient amount of a compound that fits spatially and preferentially into a ligand binding site of a melanocortin receptor of interest. By “modulating” is intended increasing or decreasing activity of a melanocortin receptor.

The subject compounds (e.g. mini-AGRPs and/or non-peptide ligands) find use in a variety of different applications, and are particularly suited for use in modulating MC3R and/or MC4R mediated physiological processes. As such, the subject compounds find use in modulating feeding behaviour, and treating disease conditions associated with feeding behaviour. For example, MCR agonists or AGRP binding antagonists of the subject methods find use inhibiting appetite, where such compounds find use in the treatment of disease conditions associated with excessive caloric intake, e.g. obesity and the like. Conversely, MCR antagonists of the subject invention find use in treating disease conditions where increasing appetite is attractive, e.g. anorexia, cachexia and the like. Other disease conditions in which the subject compounds may find use include those described in: WO 99/21517; WO 98/10068; WO 99/54358; WO 99/31508; WO 99/43709; WO 99/55679; and WO 99/57148.

Other representative uses for the subject ligands, e.g. screening assays for MCR ligands, etc., are also disclosed in these listed patent applications, the disclosures of which are herein incorporated by reference). Labeled, i.e. radio, fluorescent, biotin, antigen, etc., non-peptide ligands of melanocortin receptors find use as biological tracers for receptor identification in vivo.

The compounds of the subject invention find use in a variety of different hosts. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

The compouds of this invention are also useful as “lead compounds” for the development of therapeutics. For example, pre-clinical candidate compounds can be tested in appropriate animal models in order to measure efficacy, absorption, pharmacokinetics and toxicity following standard techniques known in the art. Compounds exhibiting desired properties are then tested in clinical trials for use in treatment of various melanocortin receptor-based disorders. These include feeding disorders, including wasting syndromes, obesity, and other disorders related to hypothalamic control of feeding. A wasting syndrome is an illness characterized by significant weight loss accompanied by other indicia of poor health, including poor appetite, gut disorder, or increased metabolic rate. Wasting syndromes include, but are not limited to, the wasting syndrome afflicting some patients diagnosed with Acquired Immune Deficiency Syndrome (AIDS) and various cancers. As methods of treating other symptoms of diseases such as AIDS progress, the incidence of wasting syndrome as the cause of death increases. Improved prophylaxis and treatment for HIV wasting syndrome is required (Kravick, et al., Arch. Intern. Med. 157:2069-2073, 1997). Anorexia and cachexia are well-known results of cancer that contribute to morbidity and mortality (Simons, et al, Cancer 82:553-560, 1998; Andrassy & Chwals, Nutrition 14:124-129, 1998). The reasons for the significant weight loss are multiple and may be directly related to the tumor, such as increased metabolic rate, but also include decreased intake due to poor appetite or gut involvement. Further, excessive leptin-like signaling may contribute to the pathogenesis of wasting illness (Schwartz, et al., Pro. Nutr. Soc. 56:785-791, 1997).

Accordingly, one aspect of the invention pertains to a method of treating a disease state in mammals that is alleviated by treatment with a polypeptide having an amino acid sequence: CX¹X²X³X⁴X⁵X⁶CX⁷X⁸X⁹X¹⁰X¹¹X¹²CCDPX¹³ ATCYCX¹⁴X¹⁵X¹⁶N AFC YCRₙ (SEQ ID NO:__), wherein X¹, X², X³, X⁴, X⁵, X⁶, X⁷, X⁸, X⁹, X¹⁰, X¹¹, X¹², X¹³, X¹⁴, X¹⁵, and n is 0 or 1, which method comprises administering to a mammal in need of such a treatment a therapeutically effective amount of the polypeptide, which can be administered, by way of illustration and not limitation, in a liquid formulations or a solid formulations, such as in the form of a pharmaceutically acceptable salt thereof. In one preferred embodiment, the polypeptide has the amino acid sequence: CVRLHESCLGQQVPCC DPAATCYCRFFNAFCYC (SEQ ID NO:3). In certain embodiments, such a disease state can be a wasting syndrome.

There are many other uses and advantages provided by the present invention. For example, the methods and compositions described herein are useful for identifying peptides, peptidomimetics or small natural or synthetic organic molecules that modulate melanocortin receptor activity. The compounds are useful in treating melanocortin receptor-based disorders. Methods and compositions of the invention also find use in characterizing structure/function relationships of natural and synthetic ligand compounds

VIII. Screening of Peptidomimetics and/or Mini-AGRPs.

A) Binding and Activity Assays.

The compounds of this invention (e.g. mini-AGRPs or peptidomimetics) or libraries thereof can be screened to identify those having a particular receptor specificity, and/or avidity, and/or activity (e.g. agonist, antagonist, etc.). Such screening methods can readily identify “lead compounds” displaying the desired properties. Once lead compounds are identified, a variety of homologs and analogs can be prepared, e.g. to facilitate an understanding of the relationship between chemical structure and biological or functional activity. These studies define structure activity relationships which are then used to design drugs with improved potency, selectivity and pharmacokinetic properties. Combinatorial chemistry can also be used to rapidly generate a variety of structures for lead optimization.

In preferred embodiments, such assays include binding assays and/or activity assays. Binding assays typically measure the ability of the ligand(s) of interest to bind a melanocortin receptor (e.g. MC3r, MC4r, etc.). The assays can be qualitative (positive or negative) or quantitative, e.g. provide a measure of binding affinity.

Suitable binding assays are well known to those of skill in the art. In a preferred embodiment, the binding assay is a competitive assay that measures the ability of the test composition to displace/compete a reference ligand from a melanocortin receptor. In one preferred approach specific binding of ligands of this invention to cells expressing the MC-3 receptor is determined by competition experiments using labeled (e.g. ¹²⁵I-labeled) Nle⁴-D-Phe⁷-α-MSH (NDP-MSH), as described in Tatro et al. (1990) Cancer Res. 50: 1237-1242).

In various embodiments, screening can be in vitro and/or in vivo. Certain preferred assays include cell-free competition assays and cell culture based assays.

Activity assays measure the ability of the compounds of this invention to activate (agonize) or inhibit (antagonize) activity at one or more melanocortin receptors or to block or augment the activity of known antagonists or antagonists. Activity assays for melanocortin receptors are well known to those of skill in the art.

Preferred assays measure, directly, or indirectly, melanocortin receptor induced changes in intracellular cAMP concentrations. One such assay measures the ability of cAMP to displace (8-³H) cAMP from a high affinity cAMP binding protein (see Gilman (1979) Proc. Natl. Acad. Sci., USA, 67: 305-312). Briefly, test cells are exposed to the ligands to be screened. Following treatment, the cells are washed twice with phosphate buffered saline and intracellular cAMP extracted by lysing the cells with 1 mL of 60% ethanol. The assay is then run as described by Gilman, supra.).

Another convenient assay system is described in U.S. Pat. No. 6,100,048. This patent discloses recombinant expression constructs comprising nucleic acid encoding mammalian melanocortin receptors, and mammalian cells into which the recombinant expression constructs have been introduced that express functional mammalian melanocortin receptors. A panel is provided of such transformed mammalian cells expressing melanocortin receptors for screening compounds for receptor agonist and antagonist activity. One typical panel includes MCir, MC2r, MC3r, and MC4r. In various embodiments, each of the cells of the panel of mammalian cells expressing mammalian melanocortin receptors further comprises a recombinant expression construct encoding a cyclic AMP responsive element (CRE) transcription factor binding site that is operatively linked to a nucleic acid sequence encoding a protein capable of producing a detectable metabolite. Expression of the protein that produces the detectable metabolite is dependent on binding of the test compound to the melanocortin receptor expressed by each cell in the panel and the intracellular production of cAMP as a result. Detection of the metabolite thereby provides a measure of the agonist or antagonist activity of the ligand(s) in question.

In a preferred embodiment, compounds of the invention bind to a melanocortin receptor ligand binding site with greater affinity than the native cellular ligand proteins. Preferred compounds show at least a 1.25 fold greater affinity, preferably at least a 1.5 fold greater affinity, more preferably at least a 2 fold greater affinity, and most preferably at least a 5 fold or 10 fold greater affinity than the native cellular ligand proteins.

Preferred compounds can additionally or alternatively, show a different receptor specificity, e.g. specificity for MC4r rather than specificity for MC3r and MC4r, etc. The compounds selected can have agonist and/or antagonistic properties. The compounds also include those that exhibit new properties with varying mixtures of agonist and antagonist activities, depending on the effects of altering ligand binding in the context of different activities of melanocortin receptors which are mediated by proteins other than ligands, and which interact with the receptors at locations other than the ligand binding site. The compounds also include those, which through their binding to receptor locations that are conformationally sensitive to ligand binding, have allosteric effects on the receptor by stabilizing or destabilizing the ligand-bound conformation of the receptor, or by directly inducing the same, similar, or different conformational changes induced in the receptor by ligand binding.

The assays described herein are meant to be illustrative and not limiting. Using the teaching provided herein, other suitable assays will be apparent to those of skill in the art.

In certain preferred embodiments, the compounds that s

B) High Throughput Screening

The activity or binding assays of this invention are also amenable to “high-throughput” modalities. As described above preferred ligands of this invention bind to melanocortin receptors and thereby modulate receptor activity. Preferred assays detect MC receptor binding and/or activity. High throughput assays for the presence, absence, or quantification of particular products are well known to those of skill in the art. Similarly, binding assays are similarly well known. Thus, for example, U.S. Pat. Nos. 5,559,410 and 5,585,639 discloses high throughput binding assays.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

It is noted that HTS can be efficiently accomplished by screening combinations of ligands in a single assay. Thus, for example, a 680 well microtitre plate could contain 100 different ligands per well providing 68,000 different ligands per plate. Those wells that show a positive score for a particular assay are then sequentially subsampled to identify the particular ligand(s) providing the positive signal.

C) Databases of Melanocortin Receptor Modulators.

In certain embodiments, the agents (ligands) that score positively in the assays described herein (e.g. show an ability to bind and/or modulate a melanocortin receptor) can be entered into a database of putative and/or actual modulators of Socs2 expression. Alternatively or additionally, a specificity fingerprint of a putative or actual modulator can be entered into the database.

The term database refers to a means for recording and retrieving information. In preferred embodiments, the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

IX. Methods for Identifying Receptor Binding Regions of Melanocortin Receptor Agonist and Antagonists.

The invention also includes compositions and methods for identifying receptor binding regions of melanocortin receptor agonist and antagonists, along with ligand binding sites of melanocortin receptors. The methods involve examining the surface of a polypeptide of interest to identify residues that modulate ligand binding. The residues can be identified by homology to the receptor binding region of MARP described herein. A preferred method is alignment with the residues of any polypeptide corresponding to (i.e., the same as or equivalent to) residues 1-18 of the N-terminal loop (residues 1-18 of SEQ ID NO:2), residues 19-34 of the central loop (residues 19-34 of SEQ ID NO:2) and residues 35-46 of the C-terminal loop (residues 35-46 of SEQ ID NO:2). Overlays and superpositioning with a three-dimensional model of the MARP receptor binding region, or a portion thereof that contains a receptor binding region, also can be used for this purpose. For example, melanocortin receptor agonists and antagonists identifiable by homology alignment include naturally occurring compounds or compounds structurally related to such naturally occurring compounds found in humans, along with synthetic compounds.

Alignment and/or modeling also can be used as a guide for the placement of mutations on the receptor binding region surface to characterize the nature of the ligand binding site on melanocortin receptors in the context of a cell. To destroy the ligand binding interaction, preferred mutations are to charged residues (e.g., Arg, Lys, or Glu) on the basis that bulky, surface charged residues might disrupt ligand binding, yet preserve the overall ligand structure and solubility. Mutants can be tested for ligand binding as well as the relative change in strength of the binding interaction. Ligand-dependent ligand interaction assays also can be tested for this purpose, such as those described herein.

Compounds that bind to the ligand binding site of melanocortin receptors can be identified by computational modeling and/or screening. For example, ligand agonists or antagonists can be identified by providing atomic coordinates comprising the MARP receptor binding region or portion thereof to a computerized modeling system, modeling them, and identifying compounds that mimic or match the receptor binding region and thus would be expected to fit spatially into the ligand binding site. By a “portion thereof” is intended the atomic coordinates corresponding to a sufficient number of residues or their atoms of the receptor binding region that interact with a melanocortin receptor capable of binding the region. As another example, an atomic structural model utilized for computational modeling and/or screening of compounds that mimic or match the receptor binding region and thus would be expected to fit spatially into the ligand binding site, may include a portion of atomic coordinates of amino acid residues corresponding to the region composed of residues 24-31 of the central loop (residues 24-31 of SEQ ID NO:2), or their structural and functional equivalents. An atomic model can also be designed that includes residues 19-34 of the central loop (residues 19-34 of SEQ ID NO:2), and a model can further be designed that includes residues 19-34 of the central loop (residues 19-34 of SEQ ID NO:2) and some or all of residues 1-18 of the N-terminal loop (residues 1-18 of SEQ ID NO:2). Thus, for example, the atomic coordinates provided to the modeling system can contain atoms of MARP, all or part of the receptor binding region or a subset of atoms useful in the modeling and design of compounds that mimic or match the receptor binding region.

X. Pharmaceutical Preparations

Also provided are pharmaceutical preparations of the subject non-peptide compounds. The subject compounds can be incorporated into a variety of formulations for therapeutic administration. More particularly, the compounds of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. The formulations may be designed for administration via a number of different routes, including oral, buccal, rectal, parenteral, intraperitoneal, intradermal, transdermal, intracheal, etc., administration.

In pharmaceutical dosage forms, the compounds may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unlike typical peptide formulations, the peptides of this invention comprising D-form amino acids can be administered, even orally, without protection against proteolysis by stomach acid, etc. Nevertheless, in certain embodiments, peptide delivery can be enhanced by the use of protective excipients. This is typically accomplished either by complexing the polypeptide with a composition to render it resistant to acidic and enzymatic hydrolysis or by packaging the polypeptide in an appropriately resistant carrier such as a liposome. Means of protecting polypeptides for oral delivery are well known in the art (see, e.g., U.S. Pat. No. 5,391,377 describing lipid compositions for oral delivery of therapeutic agents).

Where the ligands of this invention are polypeptides for oral administration, it is desirable to protect the polypeptide from digestion. This is readily accomplished by a variety of encapsulation technologies. One preferred encapsulation system is a “sustained-release system”.

Such sustained release systems are well known to those of skill in the art and can often maintain elevated serum half-life. In one preferred embodiment, the ProLease biodegradable microsphere delivery system for proteins and peptides (Tracy (1998) Biotechnol. Prog. 14: 108; Johnson et al. (1996) Nature Med. 2: 795; Herbert et al. (1998), Pharmaceut. Res. 15, 357) a dry powder composed of biodegradable polymeric microspheres containing the protein in a polymer matrix that can be compounded as a dry formulation with or without other agents.

The ProLease microsphere fabrication process was specifically designed to achieve a high protein encapsulation efficiency while maintaining protein integrity. The process consists of (i) preparation of freeze-dried protein particles from bulk protein by spray freeze-drying the drug solution with stabilizing excipients, (ii) preparation of a drug-polymer suspension followed by sonication or homogenization to reduce the drug particle size, (iii) production of frozen drug-polymer microspheres by atomization into liquid nitrogen, (iv) extraction of the polymer solvent with ethanol, and (v) filtration and vacuum drying to produce the final dry-powder product. The resulting powder contains the solid form of the protein, which is homogeneously and rigidly dispersed within porous polymer particles. The polymer most commonly used in the process, poly(lactide-co-glycolide) (PLG), is both biocompatible and biodegradable.

Encapsulation can be achieved at low temperatures (e.g., −40° C.). During encapsulation, the protein is maintained in the solid state in the absence of water, thus minimizing water-induced conformational mobility of the protein, preventing protein degradation reactions that include water as a reactant, and avoiding organic-aqueous interfaces where proteins may undergo denaturation. A preferred process uses solvents in which most proteins are insoluble, thus yielding high encapsulation efficiencies (e.g., greater than 95%).

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In therapeutic applications, the compositions of this invention are administered to a patient suffering from one or more symptoms of a wasting syndromes, obesity, and other disorders related to hypothalamic control of feeding. Such conditions include, but are not limited to wasting syndrome accompanying AIDS and various cancers. The compounds are administered in an amount sufficient to cure or at least partially prevent or arrest one or more symptoms of the the disease and/or its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms) the patient.

The concentration of ligand (polypeptide or peptiodmimetic) can vary widely, and will be selected primarily based on fluid volumes, viscosities, body weight and the like in accordance with the particular mode of administration selected and the patient's needs. Concentrations, however, will typically be selected to provide dosages ranging from about 0.1 mg/kg/day to about 1000 mg/kg/day, preferably from about 1 mg/kg/day to about 100 mg/kg/day, more preferably from about 5 mg/kg/day to about 50 mg/kg/day. It will be appreciated that such dosages may be varied to optimize a therapeutic regimen in a particular subject or group of subjects.

XI. Kits.

In still another embodiment, this invention provides kits for practice of the methods described herein. In certain embodiments the kits comprise a container containing one or more of the melanocortin ligands of this invention. The ligands can be labeled or unlabeled, and/or, optionally provided in a unit dosage form, and/or optionally, provided with or in a pharmacological acceptable excipient. The kits can optionally additionally include one or more detectable labels for labeling the ligand(s).

Certain preferred kits provide libraries of the ligands of this invention as described herein.

The kits can optionally include any reagents and/or apparatus to facilitate practice of the methods described herein. Such reagents and apparatus include, but are not limited to buffers, instrumentation, devices for administering the ligand(s) (e.g. syringes, etc.), microtiter plates, labeling reagents streptavidin or biotin conjugated substrates, PAGE gels, blotting membranes, reagents for detecting a signal, and the like.

In addition, the kits may include instructional materials containing directions (i.e., protocols) for the practice of the methods of this invention. Preferred instructional materials provide protocols for utilizing the kit contents for modulating melanocortin receptor activity and/or for screening for particular melanocortin receptors, and/or for treating a disease or pathological state. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

XII. Computer-based Methods.

The invention also provides for the NMR structure of the human AGRP C-terminus, preferably embodied in a computer readable form. Synthesis of MARP and concentrated solutions adequate for NMR spectroscopy are described in the examples. After the NMR structure of MARP is determined, the structural information can be used in computational methods to design synthetic agonist and antagonist compounds for the melanocortin receptors, and further structure-activity relationships can be determined through routine testing using the assays described herein and known in the art. It is expected that the structure coordinates of the NMR structure of MARP, as provided in Tables 4 and 5, will be particularly useful for solving the NMR structure of other melanocortin receptor antagonists or agonists. The coordinates in Table 4 and Table 5 represent the minimized average of the coordinates of a family of NMR structures. The minimized average was determined by (1) calculating an average from approximately 20 NMR structures found to meet the experimental NMR distance restraints and (2) subjecting this calculated average to a final round of energy minimization.

One method that may be employed for this purpose is molecular replacement. In this method, the unknown NMR structure, may be determined using the structure coordinates of this invention as provided in Table 4 and Table 5. The coordinates in Table 4 for the human AGRP C-terminus have been deposited with the Brookhaven National Laboratory Protein Data Bank, and have been assigned Brookhaven Protein Data Bank Accession Number Iqu8. The coordinates for Table 5 have been deposited with the Brookhaven National Laboratory Protein Data Bank, and have been assigned Brookhaven Protein Data Bank Accession Number 1HYK. This method will provide an accurate structural form for the unknown NMR structure more quickly and efficiently than attempting to determine such information ab initio.

One aspect of the invention is an NMR structure of the minimized agouti related protein, embodied in a computer readable media. Atomic coordinate information gleaned from the NMR structure of the invention is preferably stored and provided in the form of a machine-readable data storage medium. This medium contains information for constructing and/or manipulating an atomic model of a receptor binding region or portion thereof. For example, the machine readable data for the receptor binding region may comprise structure coordinates of amino acids corresponding to (i.e., the same as or equivalent to) residues 1-18 of the N-terminal loop, (residues 1-18 of SEQ ID NO:2), and/or residues 19-34 of the central loop (residues 19-34 of SEQ ID NO:2) and/or residues 35-46 of the C-terminal loop (residues 35-46 of SEQ ID NO:2), or a homologue of the molecule or molecular complex comprising the region. The machine readable data for the receptor binding region may comprise structure coordinates of amino acids corresponding to residues 1-18 of the N-terminal loop, (residues 1-18 of SEQ ID NO:2) and residues 19-34 of the central loop (residues 19-34 of SEQ ID NO:2). The machine readable data may also comprise residues 24-31 of the central loop (residues 24-31 of SEQ ID NO:2) and a portion of the N-terminal loop, for example residues 15 to 18 (residues 15-18 of SEQ ID NO:2), residues 7 to 18 (residues 7-18 of SEQ ID NO:2), or residues 1 to 18 (residues 1-18 of SEQ ID NO:2). The homologues comprise a receptor binding region that has a root mean square deviation from the backbone atoms of the amino acids of not more than 2.54 Å, preferably not more than 1.66 Å.

The machine-readable data storage medium can be used for interactive drug design and molecular replacement studies. For example, a data storage material is encoded with a first set of machine-readable data that can be combined with a second set of machine-readable data. For molecular replacement, the first set of data can comprise a Fourier transform of at least a portion of the structural coordinates of the AGRP C-terminus or portion thereof of interest, and the second data set comprises an X-ray diffraction pattern of a molecule such as the melanocortin receptor of interest. Using a machine programmed with instructions for using the first and second data sets a portion or all of the structure coordinates corresponding to the second data can be determined.

### EXAMPLES

The following examples are offered to illustrate, but not to limit the claimed invention.

Chemical synthesis and characterization of MARP

Abbreviations

| | |
|---|---|
| ³J_{HNα} | three bond αH-NH scalar coupling constant |
| AGRP/ART | Agouti Related Protein/Agouti Related Transcript |
| Conformational shift | experimental chemical shift-random coil chemical |
| | shift |
| DQF-COSY | two-dimensional double-quantum filtered correlation |
| | spectroscopy |
| HX | hydrogen-deuterium exchange |
| ICK | Inhibitor Cystine Knot |
| MARP | Minimized human Agouti Related Protein, residues |
| | 87-132 of human AGRP |
| MC3r/MC4r | Melanocortin Receptor 3/4 |
| MRE | Mean Residue Ellipticity |
| α-MSH | α-Melanocyte Stimulating Hormone |
| NDP-MSH | [Nle⁴, D-Phe⁷]α-MSH, a superpotent melanocortin |
| | agonist |
| nOe | melanocortin Overhauser enhancement |
| NOESY | two-dimensional melanocortin Overhauser |
| | spectroscopy |
| TOCSY | two-dimensional total correlation spectroscopy |

### Methods and Materials

A) Chemical Protein Synthesis:

N^{α}-Acetyl-MARP (SEQ ID NO:2) and N⁶⁰ -acetyl-MARP(Arg25Ala) were synthesized, folded and purified to each give a protein containing five disulfide bonds, as reported in Yang, et al., supra.

B) CD Experiments:

Circular dichroism (“CD”) spectra were recorded at 25° C. on an Aviv 60DS spectropolarimeter in a rectangular 1 mm path length cuvet for concentrations up to 60 μM. For concentrations higher than this a round cell with a 0.1 mm path length was used. All CD samples were 50 mM potassium phosphate, pH 4.25. Concentration dependence was ruled out in the range 20 μM-1 mM. Temperature dependence was determined for 5-85° C. The spectra are superimposable from 5 to 45° C.

C) NMR Sample Preparation:

The activity of MARP used for the NMR sample and that of a single mutant were assayed by measuring the inhibition of cAMP production in the presence of NDP-MSH (Shutter, et al., supra; Huszar, et al., supra) in HEK-293 cells transfected with human MC4r. Control experiments were performed with no MARP. The NMR samples were found to be native-like with complete activity. NMR samples contained approximately 1.9 mM MARP at pH 4.2 in 50 mM KH₂P0₄ buffer in 90% H₂O/10% D₂O. Additional samples for HX experiments were prepared by lyophilization of protonated samples followed by reconstitution in 700 μl D₂O.

D) NMR Experiments:

¹H 2D NMR spectra were principally acquired at 15° C. on a Varian 500 Unity Plus spectrometer using inverse probes. Initial NMR data were routinely acquired with a 6000 Hz spectral width, 4096 complex points in t₂, and 512 (TOCSY/IDQF-COSY) or 700 (NOESY) increments in t₁. All spectra were processed using the MN package (Carlsberg Laboratory, Department of Chemistry, Denmark) and analyzed using XEASY (Bartels, et al., J. Biomol. NMR 5:1-10 (1995)), with chemical shifts referenced to 1.4-dioxime at 3.743 ppm. Sequential assignments of all backbone and >90% of side chain protons were accomplished using standard methods (Wüthrich, K. “NMR of Proteins and Nucleic Acids” (John Wiley and Sons, New York, 1986); Redfield, C. in “NMR of Macromolecules: A Practical Approach, pp. 71-99 (Roberts, G. K. C., Ed., IRL Press at Oxford University Press, Oxford, 1993)) for 50 ms TOCSY, 150 ms NOESY and DQF-COSY data. Additional data sets were acquired at 25° C. and 30° C. to resolve ambiguities. Examination of the three Pro residues identified nOes consistent only with trans-Pro. The same experiments were repeated at 800 mHz.

Four additional peaks were identified in the αN region of the TOCSY spectrum, however, associated spin systems could not be identified and neither could nOes to the peaks in question.

NOESY data for distance restraints were collected at 15° C. using the WET sequence (Smallcombe, et al., J. Magn. Reson. Ser. A 117:295-303 (1995)) for water suppression, 1.6 s recycle delay and a mixing time of 80 ms in both H₂O and D₂O. ³J_{HNα} coupling constants at 25° C. were determined by both linear least squares fitting of the antiphase doublets in a DQF-COSY and also using the INFIT (Szyperski, et al., J. Magn. Reson. 99:552-560 (1992)) module of XEASY with 150 ms NOESY data. These methods agreed to within ±0.5 Hz for all of the measured coupling constants. At 15° C. larger intrinsic linewidths precluded accurate measurement of ³J_{HNα}.

For amide exchange experiments, the magnet was preshimmed on a 21 residue peptide sample at pH 4 in D₂O/phosphate buffer. The first TOCSY experiment was begun 23 minutes after reconstituting the protonated sample in D₂O. Four TOCSY experiments identical to those described above, except for the number of t₁ increments, were acquired back to back at 15° C. over a period of 24 hours. The first three consisted of 150 t₁ increments and the final experiment 300 increments. NOESY and DQF-COSY spectra were also acquired as described above.

E. Structure Calculations:

Final structure calculations included the covalent connectivity of the published disulfide map (Bures, et al., supra), and were based on a total of 414 interproton distance constraints derived from the 80 ms 2D NOESY spectra and 34 backbone Φ dihedral angle constraints derived from coupling constant measurements, giving a total of 448 total restraints, or 9.7 restraints per residue. The distance restraints can be broken down into 228 intraresidue (backbone to side chain only), 129 sequential, 20 medium range (1<₁i−j₁≦5) and 37 long range (₁i−j₁>5) restraints. These restraints were assigned as strong, medium or weak. The total numbers of restraints in each category were 95 strong, 246 medium, and 77 weak. All categories had a lower limit of 1.6 Å, with upper limits of 2.8, 3.5 and 5 Å for the strong, medium and weak categories, respectively. Trial structures were generated using the simulated annealing protocol from CNS version 0.4a (anneal.inp) with SUM averaging for the nOe distances (Brunger, et al., Acta Crystallogr. Sect. D Biol. Crystallogr. 54:905-921 (1998); Nilges, et al., FEBS Lett. 239:129-136 (1988); Nilges, et al., Protein Eng. 2:27-38 (1988); Kuszewski, et al., J. Magn. Reson. Ser. B 112:79-81 (1996); Stein, et al., J. Magn. Reson. 124:154-164 (1997)). Twenty structures with no bond or nOe angle violations were used to represent the solution structure of MARP (see Table 1 for Root Mean Square Deviations, “RMSDs”). Structures were displayed using MOLMOL (Koradi, et al., Science 278:135-138 (1997)).

### Results and Discussion

The synthesis and biochemical characterization of MARP were previously reported in Yang, et al., supra. The N-terminal residue (Cys-1) of MARP corresponds to the first Cys (Cys-87) of the Cys-rich region in full length, 132 residue human AGRP. The material used for the MARP NMR sample showed native-like activity as measured by its ability to competitively inhibit NDP-MSH at MC4r, as has been shown in previous studies (Yang, et al., supra; Ollmann, et al., Science 278:135-138 (1997)). Previous mutational studies of agouti and AGRP showed residues Arg-25, Phe-26, Phe-27 (the RFF triplet, Tota, et al., supra) and Asp-17 (in agouti) to be determinants of receptor binding (Kiefer, et al., Biochemistry 36:2084-2090 (1997); Kiefer, et al., Biochemistry 37:991-997 (1998); Tota, et al., supra)).

This experiment measured inhibition of NDP-MSH stimulated cAMP generation in cells transfected with MC4r. A control experiment was conducted with no MARP present. The addition of MARP (5.0×10⁻⁶ M) demonstrated competitive inhibition of NDP-MSH. The addition of MARP with Arg-25 to Ala substitution (5.0×10⁻⁶ M) demonstrated loss of inhibition due to a mutation in the active loop. This experiment thus showed that the replacement of Arg-25 by Ala results in a complete loss of inhibitory activity.

The far-UV CD spectrum of MARP was measured in 50 mM phosphate buffer at pH 4.25 as a function of temperature with spectra shown every 5° C. from 5° C. to 30° C. and also at 45° C. The spectrum was similar to that reported for a similar C-terminal fragment (residues 85-132) of AGRP (residues 85-132 of SEQ ID NO:1) reported by Rosenfeld, et al., supra, and is characterized by a negative maximum at approximately 198 nm and a slight negative maximum at 245 nm. There was little indication of canonical α-helix, however the spectrum did suggest some β character or turns. The near-UV CD spectrum, indicative of tertiary structure, showed a weak minimum at approximately 275 nm which could be attributed to the disulfide bonds and possibly restricted orientations of the side chains of the Tyr-23 and Tyr-32.

At 25° C. between 20 μM and 1.0 mM there was no observable concentration dependence as measured by CD. Between 1 mM and 1.9 mM there were no concentration dependent changes in the NMR spectra (i.e. linewidths, chemical shifts, etc. all remain constant). The far-UV CD spectrum remained constant between 5° C. and 45° C., a temperature range well beyond that of the present NMR experiments. Thus, by all indications, MARP existed as a monomer and did not exhibit temperature dependence under the conditions of the NMR experiments. The characteristics of the NMR spectra were indicative of a well folded protein with a single predominant conformer. ³J_{HNα} coupling constants and temperature coefficient also indicated a fully folded, non-random coil conformer as shown in Table 1 below, which shows the MARP coupling constants at 25° C. and NH temperature coefficients in 50 min phosphate buffer at pH 4.2.

TABLE 1
³J_{NHα} coupling constants and temperature coefficients.

| | | NH temperature coefficient |
|---|---|---|
| Residue | ³J_{NHα} coupling constant (Hz) | (ppb/° C.) |
| Cys-1 | | 6.93 |
| Val-2 | 8.79 | 3.63 |
| Arg-3 | 7.00 | 6.37 |
| Leu-4 | 5.64 | 3.47 |
| His-5 | 7.50 | 2.58 |
| Glu-6 | 9.34 | 2.90 |
| Ser-7 | | 5.86 |
| Cys-8 | 9.92 | 3.84 |
| Leu-9 | 4.40 | 2.43 |
| Gly-10 | | 7.06 |
| Gln-11 | 8.14 | 4.94 |
| Gln-12 | 8.00 | 4.38 |
| Val-13 | 9.03 | 5.56 |
| Pro-14 | | 0.00 |
| Cys-15 | 4.33 | 6.74 |
| Cys-16 | 5.42 | 3.10 |
| Asp-17 | | 4.81 |
| Pro-18 | | 0.00 |
| Cys-19 | 8.81 | 3.84 |
| Ala-20 | 9.64 | 3.23 |
| Thr-21 | 9.36 | 4.04 |
| Cys-22 | 5.76 | 7.56 |
| Tyr-23 | | 2.01 |
| Cys-24 | | 3.14 |
| Arg-25 | 5.90 | 2.93 |
| Phe-26 | 8.70 | 3.67 |
| Phe-27 | 3.68 | 6.21 |
| Asn-28 | 7.82 | 5.66 |
| Ala-29 | 7.56 | 2.10 |
| Phe-30 | 7.59 | 4.84 |
| Cys-31 | 9.17 | 5.84 |
| Tyr-32 | 9.49 | 4.43 |
| Cys-33 | | 1.13 |
| Arg-34 | 9.34 | 2.74 |
| Lys-35 | 7.25 | 8.80 |
| Leu-36 | 7.74 | 6.43 |
| Gly-37 | | 7.00 |
| Thr-38 | 8.07 | 2.07 |
| Ala-39 | 4.48 | 7.67 |
| Met-40 | 7.50 | 2.98 |
| Asn-41 | 8.19 | 1.08 |
| Pro-42 | | 0.00 |
| Cys-43 | 7.24 | 7.86 |
| Ser-44 | 7.26 | 8.38 |
| Arg-45 | 7.54 | 4.47 |
| Thr-46 | | 4.61 |

### Example 2

### Structural Description of MARP

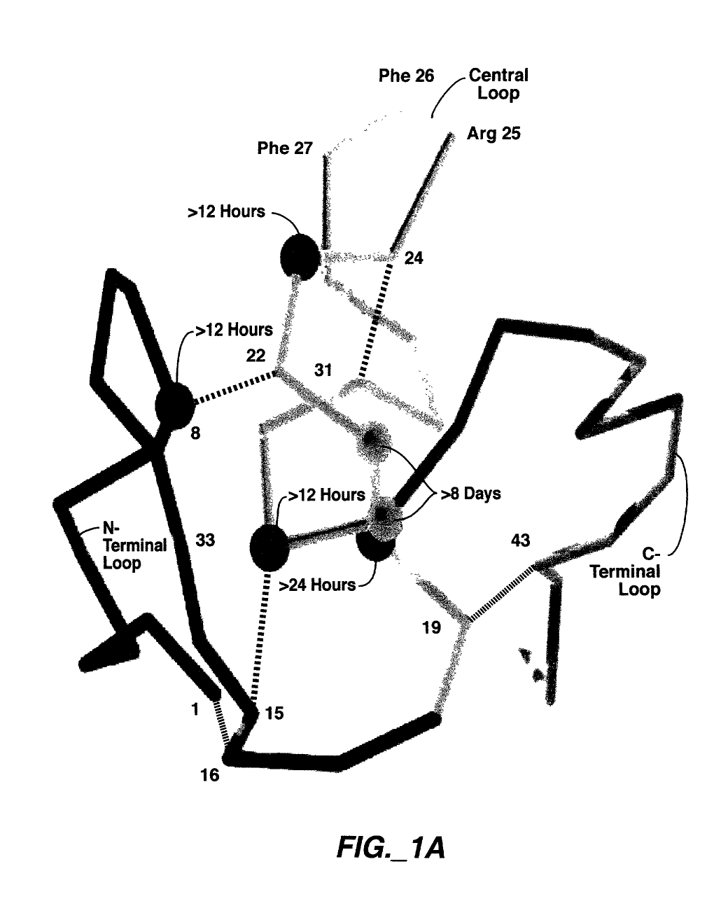

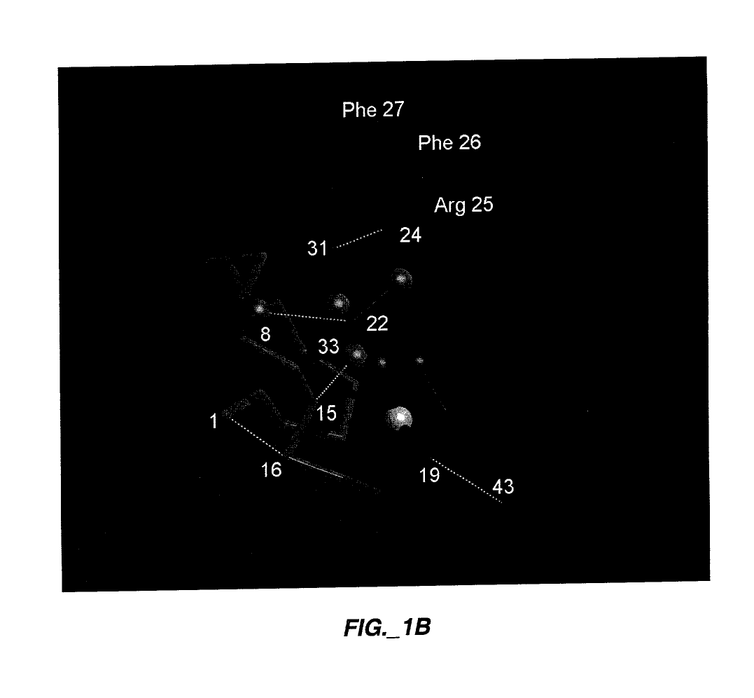

The minimized average NMR structure of MARP is shown in FIG. 1. Consistent with the far-UV CD spectrum, MARP showed little evidence of helical or regular sheet secondary structure. The disulfide bonds (1-16, 8-22, 15-33, 19-43 and 24-31) appeared to form a scaffold upon which the structure was apportioned into three major loops, which are referred to as the N-terminal loop (residues 1-18), the central loop (residues 19-34) and the C-terminal loop (residues 35-46), and are indicated in FIG. 1. RMSDs for the individual loops are reported in Table 2 below:

TABLE 2
Summary of MARP backbone and heavy atom RMSDs

| | Backbone RMSD* | Heavy atom RMSD^{a} |
|---|---|---|
| Region (residues) | (Å) | (Å) |
| Global (1-46) | 2.54 | 3.26 |
| N- and active loops (1-34) | 1.66 | 2.38 |
| N-terminal loop (1-18) | 1.31 | 2.03 |
| Central loop (19-34) | 1.51 | 2.22 |
| Active loop (24-31) | 0.69 | 1.53 |
| C-terminal loop (35-46) | 2.36 | 3.43 |

*Determined by fitting the family of 20 NMR structures to the minimized average structure

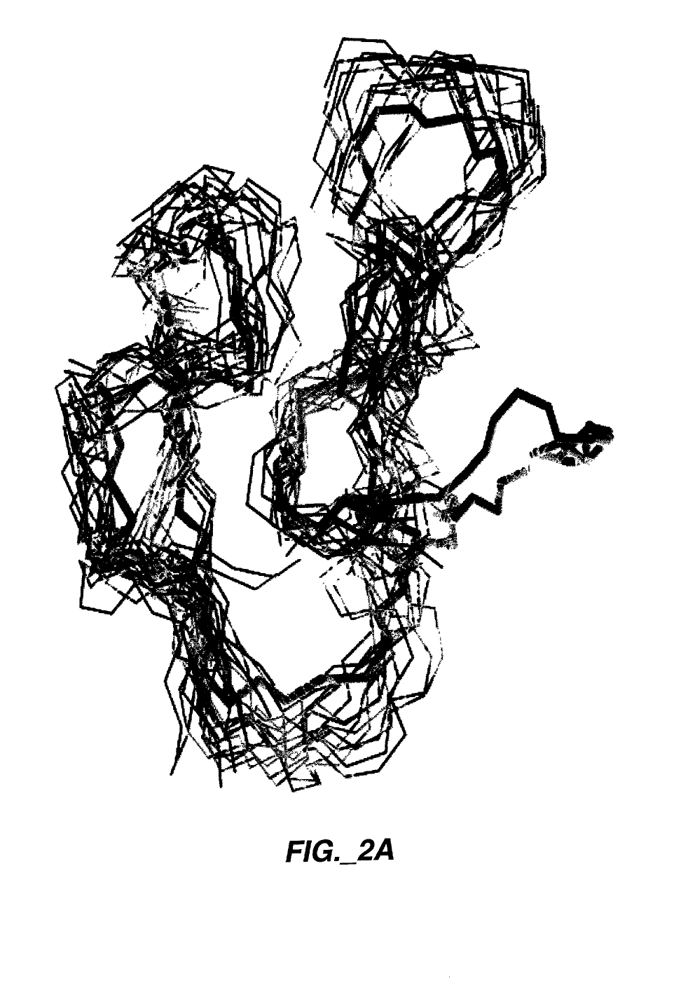

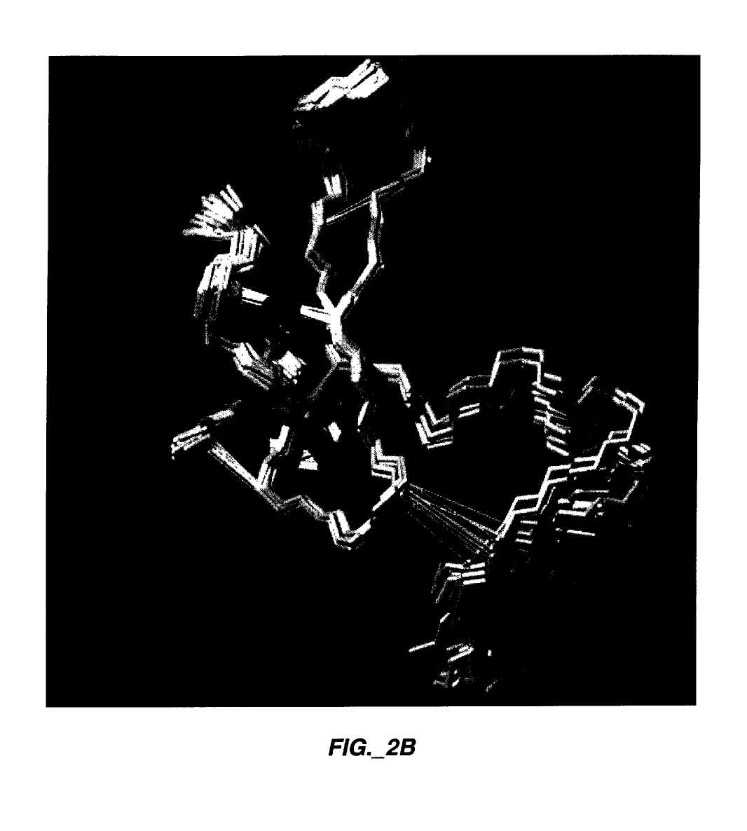

The N-terminal and central loops were much better defined both within the loops and with respect to each other than the C-terminal loop. The backbone RMSD for the entire protein (2.54 Å) was of the same order as that of the C-terminal loop (2.36 Å), while the backbone RMSD for residues 1-34 (1.66 Å) was of the order of the individual N-terminal and central loops. To demonstrate limited backbone structure variability of the N-terminal and central loops, a superposition of 14 structures (selected for clarity) for residues 1-34 and the MARP minimized average structure (residues 1-46) is shown in FIG. 2.

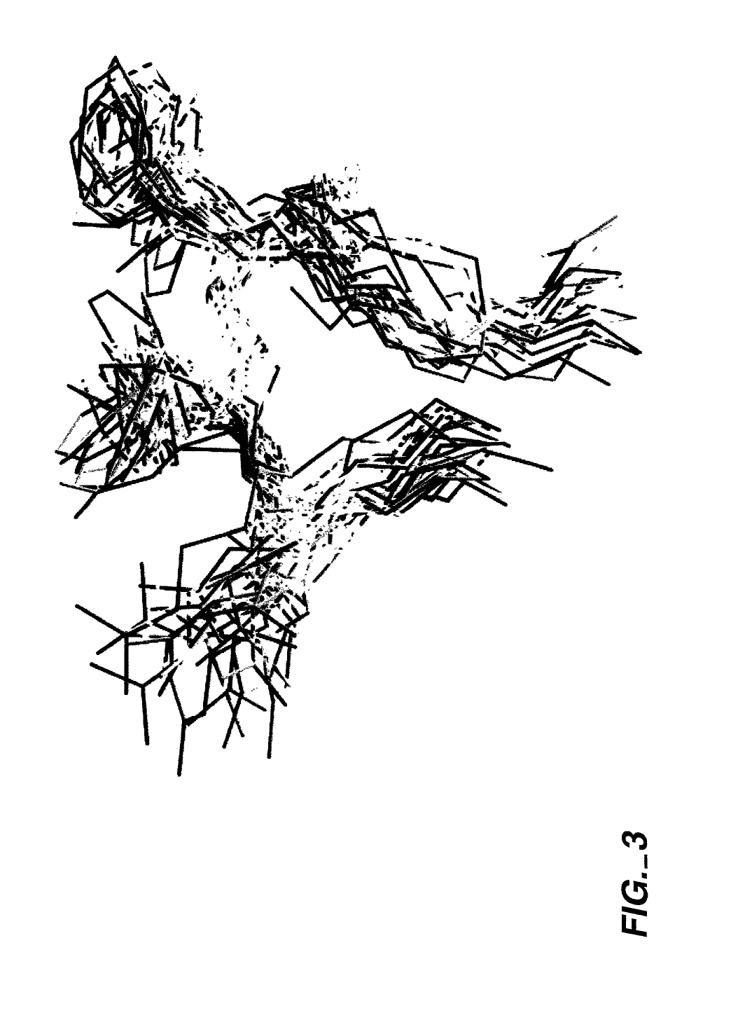

Four of the five disulfide bonds are located at the base of the structure where they appear to pinch together the bottoms of the loops to form the “core” of the protein (FIG. 1). The exception is disulfide bond 24-31 which stabilizes the central loop. The central loop, residues 19-34, contains the RFF triplet determined to be critical for activity. This motif is situated within an even smaller, well defined loop bound by Cys-24 and Cys-31 which is referred to as the “active” loop. The side chain atoms of the RFF triplet residues are located at the surface of the protein as depicted in FIG. 3. Recent experiments further highlight the importance of this active loop. These studies demonstrate that short cyclic peptides corresponding to residues 24-31 of human AGRP do in fact antagonize MC3r and MC4r (Tota, et al., supra).

Inspection of the family of NMR structures and consideration of the observed HX revealed a structure for the central loop that is best described as an irregular hairpin with a well defined loop from Cys-24 to Cys-31 (RMSD 0.6 Å, FIG. 3) and a stem region which is both twisted around and curved along its z-axis (FIG. 1). This characterization was supported by critical examination of the nOe, ³J_{HNα} and chemical shift data. As shown in FIG. 3, the active loop is highly constrained with the RFF triplet side chains exposed to solvent. Arg-25 and Phe-27 point out into the solvent, while one face of the Phe-26 aromatic ring rests parallel against the surface of the protein. Though the active loop satisfies several of the determinants for an Ω-loop (Leszczynski, et al. (1986) Science 234:849-855), the side chain orientation of Arg-25 and Phe-27 precludes its definition as such since Ω-loop side chains generally pack within the loop of backbone atoms.

HX experiments demonstrated that the amide protons of residues Cys-8, Ala-20, Thr-1, Tyr-23, Tyr-2, Cys-3 and Arg-34 are protected from exchange with solvent. To explore whether these results were consistent with the average structure, the program DSSP (Kabsch, et al., Biopolymers 22:2577-2637 (1983)) was used to identify potential hydrogen bonds. DSSP identified the backbone amides of Ala-20, Thr-21, Tyr-23, and Arg-34 as potential hydrogen bond donors. In addition, solvent accessible surface area calculations showed that residues Cys-8 and Cys-33 were completely buried from solvent, though in the D₂O spectrum the αN crosspeaks of these two residues overlap, thus their individual protection from exchange is uncertain. Tyr-32 had only 8% solvent accessible surface area at the C_{δ}protons.

The NMR structure (Table 4) gave a well resolved fold, however, as mentioned previously, canonical helices and β-sheets were not identified on the basis of nOes or other protocols including the chemical shift index (Wishart, et al., Biochemistry 31:1647-1651 (1992)) or ³J_{HNα} coupling constants (Wüthrich, K., supra).

The guidelines for these protocols assigned secondary structure on the basis of four or more consecutive residues with similar conformational shifts or ³J_{HNα}. Helical structure was characterized by 3J_{HNα}<6 Hz and negative conformational shifts and β-sheet by ³J_{HNα}>8 Hz and positive conformational shifts. Even in the active loop and stem region of the central loop, no regular secondary structure was identified by these criteria. However, the chemical shift index pointed towards a possible extended strand from residue 31 to residue 35. Table 3 sets forth the ¹H chemical shifts at 15° C., ³J_{NHα}, at 250° C., and the NH temperature coefficients.

TABLE 3
MARP ¹H chemical shifts at 15° C. in 50 mm phosphate buffer at
pH 4.2

| Residue | NH (ppm) | H_{α} (ppm) | H_{β} (ppm) | Other (ppm) |
|---|---|---|---|---|
| Cys-1 | 8.00 | 5.00 | 3.12, 2.81 | |
| Val-2 | 8.92 | 4.18 | 1.94, 2.08 | CH^{γ}₃ 0.91, 0.80 |
| Arg-3 | 8.91 | 4.01 | 1.67, 1.95 | H^{γ}1.69, 1.82, H^{δ}3.24, 3.26, |
| | | | | NH 7.31 |
| Leu-4 | 8.13 | 3.72 | 1.34, 1.54 | H^{γ}1.03, CH^{δ}₃ 0.79 |
| His-5 | 8.99 | 4.25 | 3.70, 1.42 | H^{δ}7.22, ^{Hε}8.51 |
| Glu-6 | 8.04 | 4.66 | 2.16 | H^{γ}2.24 |
| Ser-7 | 8.63 | 4.68 | 3.84 | |
| Cys-8 | 8.08 | 4.96 | 3.57, 3.13 | |
| Leu-9 | 7.92 | 4.08 | 1.57, 1.44 | H^{γ}1.44, CH^{δ}₃0.82 |
| Gly-10 | 8.78 | 4.07, 3.69 | | |
| Gln-11 | 8.06 | 4.24 | 1.95 | H^{γ} 2.28. H^{ε2} 6.86, 7.49 |
| Gln-12 | 8.68 | 4.27 | 2.20, 1.95 | H^{γ} 2.30, 2.34, H^{ε2} 6.82, |
| Val-13 | 7.48 | 4.47 | 2.05 | 7.45 CH^{γ}₃ 0.79, 0.90 |
| Pro-14 | 8.81 | 4.55 | 2.25, 1.99 | H^{γ} 1.84, 1.99, H^{δ} 3.65, |
| | | | | 3.77 |
| Cys-15 | | 4.92 | 3.35, 1.74 | |
| Cys-16 | 9.58 | 4.22 | 2.62, 3.17 | |
| Asp-17 | 8.19 | 4.78 | 2.63, 2.41 | |
| Pro-18 | 8.88 | 4.50 | 2.35 | H^{γ} 1.98, H^{δ} 3.89, 4.04 |
| Cys-19 | | 4.72 | 2.94, 3.47 | |
| Ala-20 | 8.01 | 4.94 | 1.26 | |
| Thr-21 | 8.76 | 4.58 | 4.00 | CH^{γ}₃ 1.16 |
| Cys-22 | 8.94 | 4.58 | 2.94, 3.02 | |
| Tyr-23 | 8.71 | 4.62 | 2.79 | H^{δ} 6.93, H^{ε} 6.74 |
| Cys-24 | 8.30 | 4.91 | 3.23, 2.60 | |
| Arg-25 | 8.29 | 3.84 | 1.83, 1.55 | H^{γ}1.56, 1.23, H^{δ} 3.06 |
| Phe-26 | 7.91 | 4.74 | 2.80, 3.32 | H^{δ} 7.26, H^{ε} 7.42 |
| Phe-27 | 8.54 | 4.19 | 3.12, 3.02 | H^{δ} 7.17, H^{ε} 7.33 |
| Asn-28 | 8.48 | 4.20 | 2.39, 7.77 | H^{δ2} 6.66, 7.30 |
| Ala-29 | 7.75 | 4.38 | 1.25 | |
| Phe-30 | 8.38 | 4.21 | 3.32, 3.36 | |
| Cys-31 | 8.34 | 5.63 | 2.59, 3.03 | |
| Tyr-32 | 8.88 | 5.20 | 2.59, 2.81 | H^{δ} 6.92, H^{ε} 6.66 |
| Cys-33 | 8.19 | 4.96 | 3.21, 2.66 | |
| Arg-34 | 9.43 | 4.75 | 1.83, 1.70 | H^{γ}1.57, 1.69, H^{δ} 2.62, |
| | | | | 2.88, NH 7.08 |
| Lys-35 | 9.09 | 4.47 | 1.82, 1.68 | H^{γ}1.25, 1.43, H^{δ} 1.64, |
| | | | | H^{ε} 2.89 |
| Leu-36 | 8.77 | 4.3640 | 1.63 | H^{γ}1.52, CH^{δ}₃ 0.82, 0.70 |
| Gly-37 | 8.48 | 4.09, 4.01 | | |
| Thr-38 | 7.82 | 4.42 | 4.42 | CH^{γ}₃ 1.21 |
| Ala-39 | 8.53 | 4.16 | 1.42 | |
| Met-40 | 8.06 | 4.40 | 1.95, 2.09 | H^{γ}2.51, 2.62 |
| Asn-41 | 7.74 | 5.00 | 2.62, 2.81 | H^{δ2} 7.67, 6.95 |
| Pro-42 | 8.68 | 4.47 | 2.29 | H^{γ}1.94, H^{δ} 3.61, 3.67 |
| Cys-43 | | 4.59 | 3.22, 3.13 | |
| Ser-44 | 8.37 | 4.46 | 3.86 | |
| Arg-45 | 8.26 | 4.43 | 1.93 | H^{γ}1.66, H^{δ} 3.21, NH 7.25 |
| Thr-46 | 7.86 | 4.15 | 4.23 | CH^{γ}₃1.15 |

TABLE 4
MARP atomic coordinates in 3 dimensional space
determine by NMR at 500 mHz.

| | | | | | | | | | |
|---|---|---|---|---|---|---|---|---|---|
| ATOM | 1 | CA | CYS | 1 | −6.900 | 2.269 | −8.250 | 1.00 | 4.62 |
| ATOM | 2 | HA | CYS | 1 | −5.897 | 2.041 | −7.920 | 1.00 | 4.59 |
| ATOM | 3 | HB1 | CYS | 1 | −7.160 | 4.143 | −9.269 | 1.00 | 5.13 |
| ATOM | 4 | HB2 | CYS | 1 | −6.230 | 4.254 | −7.780 | 1.00 | 4.63 |
| ATOM | 5 | C | CYS | 1 | −7.900 | 1.623 | −7.293 | 1.00 | 3.97 |
| ATOM | 6 | O | CYS | 1 | −9.067 | 1.425 | −7.636 | 1.00 | 4.16 |
| ATOM | 7 | CB | CYS | 1 | −7.088 | 3.796 | −8.249 | 1.00 | 4.78 |
| ATOM | 8 | SG | CYS | 1 | −8.576 | 4.376 | −7.358 | 1.00 | 5.12 |
| ATOM | 9 | N | CYS | 1 | −7.069 | 1.766 | −9.642 | 1.00 | 5.38 |
| ATOM | 10 | HT1 | CYS | 1 | −8.026 | 2.021 | −9.958 | 1.00 | 5.84 |
| ATOM | 11 | HT2 | CYS | 1 | −6.942 | 0.734 | −9.624 | 1.00 | 5.52 |
| ATOM | 12 | HT3 | CYS | 1 | −6.346 | 2.222 | −10.234 | 1.00 | 5.55 |
| ATOM | 13 | N | VAL | 2 | −7.445 | 1.356 | −6.070 | 1.00 | 3.32 |
| ATOM | 14 | HN | VAL | 2 | −6.517 | 1.593 | −5.848 | 1.00 | 3.27 |
| ATOM | 15 | CA | VAL | 2 | −8.315 | 0.801 | −5.028 | 1.00 | 2.84 |
| ATOM | 16 | HA | VAL | 2 | −9.187 | 0.372 | −5.505 | 1.00 | 3.11 |
| ATOM | 17 | CB | VAL | 2 | −7.624 | −0.292 | −4.151 | 1.00 | 2.58 |
| ATOM | 18 | HB | VAL | 2 | −6.927 | 0.197 | −3.490 | 1.00 | 2.54 |
| ATOM | 19 | CG1 | VAL | 2 | −8.637 | −1.028 | −3.287 | 1.00 | 2.26 |
| ATOM | 20 | HG11 | VAL | 2 | −9.533 | −1.215 | −3.860 | 1.00 | 2.67 |
| ATOM | 21 | HG12 | VAL | 2 | −8.879 | −0.427 | −2.423 | 1.00 | 2.47 |
| ATOM | 22 | HG13 | VAL | 2 | −8.214 | −1.969 | −2.962 | 1.00 | 2.24 |
| ATOM | 23 | CG2 | VAL | 2 | −6.851 | −1.295 | −5.000 | 1.00 | 3.27 |
| ATOM | 24 | HG21 | VAL | 2 | −6.240 | −1.922 | −4.350 | 1.00 | 3.49 |
| ATOM | 25 | HG22 | VAL | 2 | −6.214 | −0.766 | −5.692 | 1.00 | 3.65 |
| ATOM | 26 | HG23 | VAL | 2 | −7.544 | −1.914 | −5.548 | 1.00 | 3.65 |
| ATOM | 27 | C | VAL | 2 | −8.750 | 1.967 | −4.145 | 1.00 | 2.69 |
| ATOM | 28 | O | VAL | 2 | −8.214 | 3.071 | −4.285 | 1.00 | 3.12 |
| ATOM | 29 | N | ARG | 3 | −9.694 | 1.759 | −3.238 | 1.00 | 2.64 |
| ATOM | 30 | HN | ARG | 3 | −10.100 | 0.873 | −3.138 | 1.00 | 2.81 |
| ATOM | 31 | CA | ARG | 3 | −10.120 | 2.851 | −2.380 | 1.00 | 2.90 |
| ATOM | 32 | HA | ARG | 3 | −10.214 | 3.738 | −2.998 | 1.00 | 3.25 |
| ATOM | 33 | CB | ARG | 3 | −11.466 | 2.539 | −1.720 | 1.00 | 3.25 |
| ATOM | 34 | HB1 | ARG | 3 | −11.575 | 3.157 | −0.841 | 1.00 | 3.44 |
| ATOM | 35 | HB2 | ARG | 3 | −11.477 | 1.501 | −1.422 | 1.00 | 3.38 |
| ATOM | 36 | CG | ARG | 3 | −12.662 | 2.785 | −2.628 | 1.00 | 3.73 |
| ATOM | 37 | HG1 | ARG | 3 | −13.287 | 1.905 | −2.628 | 1.00 | 4.00 |
| ATOM | 38 | HG2 | ARG | 3 | −12.307 | 2.976 | −3.630 | 1.00 | 3.91 |
| ATOM | 39 | CD | ARG | 3 | −13.486 | 3.978 | −2.164 | 1.00 | 4.23 |
| ATOM | 40 | HD1 | ARG | 3 | −13.348 | 4.788 | −2.865 | 1.00 | 4.60 |
| ATOM | 41 | HD2 | ARG | 3 | −13.136 | 4.285 | −1.189 | 1.00 | 4.30 |
| ATOM | 42 | NE | ARG | 3 | −14.913 | 3.662 | −2.078 | 1.00 | 4.74 |
| ATOM | 43 | HE | ARG | 3 | −15.185 | 2.745 | −2.294 | 1.00 | 4.90 |
| ATOM | 44 | CZ | ARG | 3 | −15.856 | 4.540 | −1.726 | 1.00 | 5.32 |
| ATOM | 45 | NH1 | ARG | 3 | −15.532 | 5.796 | −1.425 | 1.00 | 5.50 |
| ATOM | 46 | HH11 | ARG | 3 | −14.578 | 6.091 | −1.459 | 1.00 | 5.28 |
| ATOM | 47 | HH12 | ARG | 3 | −16.246 | 6.446 | −1.162 | 1.00 | 6.06 |
| ATOM | 48 | NH2 | ARG | 3 | −17.129 | 4.160 | −1.677 | 1.00 | 6.01 |
| ATOM | 49 | HH21 | ARG | 3 | −17.379 | 3.219 | −1.903 | 1.00 | 6.19 |
| ATOM | 50 | HH22 | ARG | 3 | −17.837 | 4.816 | −1.413 | 1.00 | 6.50 |
| ATOM | 51 | C | ARG | 3 | −9.053 | 3.103 | −1.329 | 1.00 | 2.75 |
| ATOM | 52 | O | ARG | 3 | −8.519 | 2.163 | −0.738 | 1.00 | 2.50 |
| ATOM | 53 | N | LEU | 4 | −8.725 | 4.372 | −1.111 | 1.00 | 3.24 |
| ATOM | 54 | HN | LEU | 4 | −9.167 | 5.082 | −1.615 | 1.00 | 3.68 |
| ATOM | 55 | CA | LEU | 4 | −7.708 | 4.726 | −0.146 | 1.00 | 3.40 |
| ATOM | 56 | HA | LEU | 4 | −6.847 | 4.143 | −0.371 | 1.00 | 3.11 |
| ATOM | 57 | CB | LEU | 4 | −7.351 | 6.196 | −0.264 | 1.00 | 4.25 |
| ATOM | 58 | HB1 | LEU | 4 | −7.379 | 6.479 | −1.304 | 1.00 | 4.60 |
| ATOM | 59 | HB2 | LEU | 4 | −6.353 | 6.339 | 0.110 | 1.00 | 4.61 |
| ATOM | 60 | CG | LEU | 4 | −8.285 | 7.097 | 0.500 | 1.00 | 4.65 |
| ATOM | 61 | HG | LEU | 4 | −8.344 | 6.741 | 1.509 | 1.00 | 4.56 |
| ATOM | 62 | CD1 | LEU | 4 | −7.773 | 8.520 | 0.517 | 1.00 | 5.17 |
| ATOM | 63 | HD11 | LEU | 4 | −7.232 | 8.714 | −0.396 | 1.00 | 5.39 |
| ATOM | 64 | HD12 | LEU | 4 | −7.113 | 8.647 | 1.363 | 1.00 | 5.60 |
| ATOM | 65 | HD13 | LEU | 4 | −8.605 | 9.203 | 0.599 | 1.00 | 5.27 |
| ATOM | 66 | CD2 | LEU | 4 | −9.659 | 6.991 | −0.103 | 1.00 | 5.22 |
| ATOM | 67 | HD21 | LEU | 4 | −10.392 | 7.382 | 0.584 | 1.00 | 5.44 |
| ATOM | 68 | HD22 | LEU | 4 | −9.857 | 5.945 | −0.295 | 1.00 | 5.42 |
| ATOM | 69 | HD23 | LEU | 4 | −9.690 | 7.541 | −1.030 | 1.00 | 5.58 |
| ATOM | 70 | C | LEU | 4 | −8.186 | 4.418 | 1.274 | 1.00 | 3.37 |
| ATOM | 71 | O | LEU | 4 | −9.386 | 4.241 | 1.507 | 1.00 | 3.67 |
| ATOM | 72 | N | HIS | 5 | −7.244 | 4.346 | 2.217 | 1.00 | 3.48 |
| ATOM | 73 | HN | HIS | 5 | −6.311 | 4.491 | 1.965 | 1.00 | 3.68 |
| ATOM | 74 | CA | HIS | 5 | −7.564 | 4.044 | 3.618 | 1.00 | 3.77 |
| ATOM | 75 | HA | HIS | 5 | −6.629 | 3.939 | 4.150 | 1.00 | 3.80 |
| ATOM | 76 | CB | HIS | 5 | −8.361 | 5.193 | 4.240 | 1.00 | 4.60 |
| ATOM | 77 | HB1 | HIS | 5 | −8.651 | 4.923 | 5.245 | 1.00 | 4.82 |
| ATOM | 78 | HB2 | HIS | 5 | −9.248 | 5.373 | 3.650 | 1.00 | 4.71 |
| ATOM | 79 | CG | HIS | 5 | −7.583 | 6.467 | 4.309 | 1.00 | 5.19 |
| ATOM | 80 | ND1 | HIS | 5 | −8.108 | 7.657 | 4.768 | 1.00 | 5.95 |
| ATOM | 81 | HD1 | HIS | 5 | −9.021 | 7.792 | 5.094 | 1.00 | 6.16 |
| ATOM | 82 | CD2 | HIS | 5 | −6.300 | 6.730 | 3.966 | 1.00 | 5.27 |
| ATOM | 83 | HD2 | HIS | 5 | −5.586 | 6.017 | 3.564 | 1.00 | 4.82 |
| ATOM | 84 | CE1 | HIS | 5 | −7.182 | 8.597 | 4.702 | 1.00 | 6.46 |
| ATOM | 85 | HE1 | HIS | 5 | −7.307 | 9.629 | 4.993 | 1.00 | 7.11 |
| ATOM | 86 | NE2 | HIS | 5 | −6.076 | 8.060 | 4.221 | 1.00 | 6.10 |
| ATOM | 87 | HE2 | HIS | 5 | −5.204 | 8.506 | 4.184 | 1.00 | 6.44 |
| ATOM | 88 | C | HIS | 5 | −8.338 | 2.726 | 3.736 | 1.00 | 3.44 |
| ATOM | 89 | O | HIS | 5 | −8.921 | 2.423 | 4.779 | 1.00 | 3.81 |
| ATOM | 90 | N | GLU | 6 | −8.318 | 1.949 | 2.656 | 1.00 | 2.90 |
| ATOM | 91 | HN | GLU | 6 | −7.824 | 2.247 | 1.869 | 1.00 | 2.77 |
| ATOM | 92 | CA | GLU | 6 | −8.984 | 0.664 | 2.592 | 1.00 | 2.75 |
| ATOM | 93 | HA | GLU | 6 | −9.613 | 0.554 | 3.462 | 1.00 | 3.21 |
| ATOM | 94 | CB | GLU | 6 | −9.838 | 0.592 | 1.327 | 1.00 | 2.53 |
| ATOM | 95 | HB1 | GLU | 6 | −9.174 | 0.488 | 0.478 | 1.00 | 2.28 |
| ATOM | 96 | HB2 | GLU | 6 | −10.392 | 1.513 | 1.227 | 1.00 | 2.71 |
| ATOM | 97 | CG | GLU | 6 | −10.825 | −0.566 | 1.307 | 1.00 | 2.93 |
| ATOM | 98 | HG1 | GLU | 6 | −11.828 | −0.167 | 1.356 | 1.00 | 3.17 |
| ATOM | 99 | HG2 | GLU | 6 | −10.645 | −1.192 | 2.168 | 1.00 | 3.31 |
| ATOM | 100 | CD | GLU | 6 | −10.701 | −1.416 | 0.055 | 1.00 | 3.19 |
| ATOM | 101 | OE1 | GLU | 6 | −11.236 | −1.006 | −0.997 | 1.00 | 3.57 |
| ATOM | 102 | OE2 | GLU | 6 | −10.068 | −2.491 | 0.127 | 1.00 | 3.55 |
| ATOM | 103 | C | GLU | 6 | −7.955 | −0.456 | 2.557 | 1.00 | 2.53 |
| ATOM | 104 | O | GLU | 6 | −8.269 | −1.602 | 2.876 | 1.00 | 2.98 |
| ATOM | 105 | N | SER | 7 | −6.729 | −0.115 | 2.130 | 1.00 | 2.02 |
| ATOM | 106 | HN | SER | 7 | −6.557 | 0.801 | 1.870 | 1.00 | 1.87 |
| ATOM | 107 | CA | SER | 7 | −5.656 | −1.086 | 2.013 | 1.00 | 1.98 |
| ATOM | 108 | HA | SER | 7 | −4.753 | −0.563 | 1.693 | 1.00 | 1.74 |
| ATOM | 109 | CB | SER | 7 | −5.409 | −1.725 | 3.371 | 1.00 | 2.69 |
| ATOM | 110 | HB1 | SER | 7 | −5.970 | −2.643 | 3.442 | 1.00 | 3.00 |
| ATOM | 111 | HB2 | SER | 7 | −5.742 | −1.040 | 4.133 | 1.00 | 2.94 |
| ATOM | 112 | OG | SER | 7 | −4.036 | −2.005 | 3.572 | 1.00 | 3.30 |
| ATOM | 113 | HG | SER | 7 | −3.939 | −2.883 | 3.947 | 1.00 | 3.67 |
| ATOM | 114 | C | SER | 7 | −6.066 | −2.147 | 0.984 | 1.00 | 2.01 |
| ATOM | 115 | O | SER | 7 | −7.207 | −2.143 | 0.517 | 1.00 | 2.80 |
| ATOM | 116 | N | CYS | 8 | −5.180 | −3.074 | 0.643 | 1.00 | 1.78 |
| ATOM | 117 | HN | CYS | 8 | −4.285 | −3.074 | 1.044 | 1.00 | 1.85 |
| ATOM | 118 | CA | CYS | 8 | −5.558 | −4.119 | −0.301 | 1.00 | 2.24 |
| ATOM | 119 | HA | CYS | 8 | −6.476 | −3.820 | −0.745 | 1.00 | 2.23 |
| ATOM | 120 | HB1 | CYS | 8 | −3.606 | −4.528 | −1.025 | 1.00 | 2.70 |
| ATOM | 121 | HB2 | CYS | 8 | −4.503 | −3.311 | −1.949 | 1.00 | 2.24 |
| ATOM | 122 | C | CYS | 8 | −5.763 | −5.451 | 0.432 | 1.00 | 2.87 |
| ATOM | 123 | O | CYS | 8 | −5.575 | −6.522 | −0.140 | 1.00 | 3.43 |
| ATOM | 124 | CB | CYS | 8 | −4.568 | −4.261 | −1.425 | 1.00 | 2.52 |
| ATOM | 125 | SG | CYS | 8 | −5.047 | −5.510 | −2.659 | 1.00 | 3.51 |
| ATOM | 126 | N | LEU | 9 | −6.155 | −5.353 | 1.714 | 1.00 | 3.11 |
| ATOM | 127 | HN | LEU | 9 | −6.288 | −4.468 | 2.091 | 1.00 | 3.01 |
| ATOM | 128 | CA | LEU | 9 | −6.400 | −6.509 | 2.578 | 1.00 | 3.82 |
| ATOM | 129 | HA | LEU | 9 | −7.159 | −7.116 | 2.111 | 1.00 | 4.03 |
| ATOM | 130 | CB | LEU | 9 | −5.122 | −7.346 | 2.756 | 1.00 | 4.45 |
| ATOM | 131 | HB1 | LEU | 9 | −5.161 | −7.820 | 3.726 | 1.00 | 4.87 |
| ATOM | 132 | HB2 | LEU | 9 | −4.275 | −6.676 | 2.739 | 1.00 | 4.44 |
| ATOM | 133 | CG | LEU | 9 | −4.890 | −8.438 | 1.701 | 1.00 | 4.92 |
| ATOM | 134 | HG | LEU | 9 | −4.327 | −8.018 | 0.881 | 1.00 | 4.96 |
| ATOM | 135 | CD1 | LEU | 9 | −4.073 | −9.583 | 2.281 | 1.00 | 5.65 |
| ATOM | 136 | HD11 | LEU | 9 | −3.633 | −9.273 | 3.217 | 1.00 | 5.93 |
| ATOM | 137 | HD12 | LEU | 9 | −3.291 | −9.852 | 1.587 | 1.00 | 5.76 |
| ATOM | 138 | HD13 | LEU | 9 | −4.715 | −10.435 | 2.449 | 1.00 | 6.14 |
| ATOM | 139 | CD2 | LEU | 9 | −6.214 | −8.951 | 1.149 | 1.00 | 5.23 |
| ATOM | 140 | HD21 | LEU | 9 | −6.026 | −9.607 | 0.313 | 1.00 | 5.47 |
| ATOM | 141 | HD22 | LEU | 9 | −6.812 | −8.111 | 0.821 | 1.00 | 5.42 |
| ATOM | 142 | HD23 | LEU | 9 | −6.743 | −9.490 | 1.921 | 1.00 | 5.45 |
| ATOM | 143 | C | LEU | 9 | −6.919 | −6.051 | 3.948 | 1.00 | 3.94 |
| ATOM | 144 | O | LEU | 9 | −7.894 | −6.604 | 4.462 | 1.00 | 4.53 |
| ATOM | 145 | N | GLY | 10 | −6.267 | −5.033 | 4.530 | 1.00 | 3.63 |
| ATOM | 146 | HN | GLY | 10 | −5.501 | −4.627 | 4.073 | 1.00 | 3.32 |
| ATOM | 147 | CA | GLY | 10 | −6.681 | −4.513 | 5.824 | 1.00 | 4.02 |
| ATOM | 148 | HA1 | GLY | 10 | −5.848 | −4.575 | 6.507 | 1.00 | 4.32 |
| ATOM | 149 | HA2 | GLY | 10 | −7.490 | −5.120 | 6.203 | 1.00 | 4.54 |
| ATOM | 150 | C | GLY | 10 | −7.146 | −3.066 | 5.745 | 1.00 | 3.77 |
| ATOM | 151 | O | GLY | 10 | −8.151 | −2.771 | 5.104 | 1.00 | 4.10 |
| ATOM | 152 | N | GLN | 11 | −6.406 | −2.169 | 6.402 | 1.00 | 3.61 |
| ATOM | 153 | HN | GLN | 11 | −5.613 | −2.477 | 6.886 | 1.00 | 3.76 |
| ATOM | 154 | CA | GLN | 11 | −6.727 | −0.736 | 6.407 | 1.00 | 3.67 |
| ATOM | 155 | HA | GLN | 11 | −6.984 | −0.452 | 5.397 | 1.00 | 3.70 |
| ATOM | 156 | CB | GLN | 11 | −7.923 | −0.451 | 7.325 | 1.00 | 4.51 |
| ATOM | 157 | HB1 | GLN | 11 | −7.690 | 0.399 | 7.949 | 1.00 | 4.89 |
| ATOM | 158 | HB2 | GLN | 11 | −8.094 | −1.312 | 7.954 | 1.00 | 4.88 |
| ATOM | 159 | CG | GLN | 11 | −9.210 | −0.148 | 6.570 | 1.00 | 4.88 |
| ATOM | 160 | HG1 | GLN | 11 | −9.043 | −0.320 | 5.517 | 1.00 | 5.09 |
| ATOM | 161 | HG2 | GLN | 11 | −9.467 | 0.889 | 6.727 | 1.00 | 4.85 |
| ATOM | 162 | CD | GLN | 11 | −10.376 | −1.008 | 7.023 | 1.00 | 5.60 |
| ATOM | 163 | OE1 | GLN | 11 | −10.540 | −2.140 | 6.568 | 1.00 | 6.07 |
| ATOM | 164 | NE2 | GLN | 11 | −11.195 | −0.474 | 7.924 | 1.00 | 6.05 |
| ATOM | 165 | HE21 | GLN | 11 | −11.006 | 0.433 | 8.244 | 1.00 | 5.94 |
| ATOM | 166 | HE22 | GLN | 11 | −11.958 | −1.009 | 8.229 | 1.00 | 6.67 |
| ATOM | 167 | C | GLN | 11 | −5.510 | 0.082 | 6.852 | 1.00 | 3.45 |
| ATOM | 168 | O | GLN | 11 | −5.279 | 0.248 | 8.052 | 1.00 | 4.02 |
| ATOM | 169 | N | GLN | 12 | −4.718 | 0.570 | 5.893 | 1.00 | 2.94 |
| ATOM | 170 | HN | GLN | 12 | −4.930 | 0.397 | 4.955 | 1.00 | 2.77 |
| ATOM | 171 | CA | GLN | 12 | −3.520 | 1.338 | 6.219 | 1.00 | 3.05 |
| ATOM | 172 | HA | GLN | 12 | −3.399 | 1.297 | 7.292 | 1.00 | 3.57 |
| ATOM | 173 | CB | GLN | 12 | −2.294 | 0.679 | 5.573 | 1.00 | 3.41 |
| ATOM | 174 | HB1 | GLN | 12 | −1.434 | 1.314 | 5.730 | 1.00 | 3.57 |
| ATOM | 175 | HB2 | GLN | 12 | −2.471 | 0.583 | 4.512 | 1.00 | 3.50 |
| ATOM | 176 | CG | GLN | 12 | −1.976 | −0.700 | 6.130 | 1.00 | 4.12 |
| ATOM | 177 | HG1 | GLN | 12 | −1.131 | −1.105 | 5.591 | 1.00 | 4.31 |
| ATOM | 178 | HG2 | GLN | 12 | −2.834 | −1.340 | 5.984 | 1.00 | 4.40 |
| ATOM | 179 | CD | GLN | 12 | −1.639 | −0.671 | 7.609 | 1.00 | 4.78 |
| ATOM | 180 | OE1 | GLN | 12 | −0.584 | −0.178 | 8.007 | 1.00 | 5.27 |
| ATOM | 181 | NE2 | GLN | 12 | −2.538 | −1.199 | 8.436 | 1.00 | 5.19 |
| ATOM | 182 | HE21 | GLN | 12 | −3.359 | −1.574 | 8.052 | 1.00 | 5.11 |
| ATOM | 183 | HE22 | GLN | 12 | −2.342 | −1.191 | 9.396 | 1.00 | 5.76 |
| ATOM | 184 | C | GLN | 12 | −3.640 | 2.820 | 5.813 | 1.00 | 2.91 |
| ATOM | 185 | O | GLN | 12 | −4.438 | 3.561 | 6.388 | 1.00 | 3.44 |
| ATOM | 186 | N | VAL | 13 | −2.821 | 3.248 | 4.843 | 1.00 | 2.75 |
| ATOM | 187 | HN | VAL | 13 | −2.199 | 2.625 | 4.444 | 1.00 | 2.86 |
| ATOM | 188 | CA | VAL | 13 | −2.795 | 4.629 | 4.377 | 1.00 | 3.05 |
| ATOM | 189 | HA | VAL | 13 | −3.226 | 5.246 | 5.153 | 1.00 | 3.57 |
| ATOM | 190 | CB | VAL | 13 | −1.331 | 5.091 | 4.154 | 1.00 | 3.46 |
| ATOM | 191 | HB | VAL | 13 | −1.291 | 6.137 | 4.363 | 1.00 | 3.85 |
| ATOM | 192 | CG1 | VAL | 13 | −0.380 | 4.402 | 5.127 | 1.00 | 3.80 |
| ATOM | 193 | HG11 | VAL | 13 | −0.395 | 3.337 | 4.956 | 1.00 | 3.78 |
| ATOM | 194 | HG12 | VAL | 13 | −0.691 | 4.607 | 6.140 | 1.00 | 4.15 |
| ATOM | 195 | HG13 | VAL | 13 | 0.622 | 4.776 | 4.976 | 1.00 | 4.28 |
| ATOM | 196 | CG2 | VAL | 13 | −0.878 | 4.877 | 2.709 | 1.00 | 3.89 |
| ATOM | 197 | HG21 | VAL | 13 | −0.918 | 5.815 | 2.176 | 1.00 | 4.21 |
| ATOM | 198 | HG22 | VAL | 13 | −1.528 | 4.163 | 2.224 | 1.00 | 4.18 |
| ATOM | 199 | HG23 | VAL | 13 | 0.135 | 4.503 | 2.700 | 1.00 | 4.14 |
| ATOM | 200 | C | VAL | 13 | −3.615 | 4.813 | 3.090 | 1.00 | 3.04 |
| ATOM | 201 | O | VAL | 13 | −4.211 | 3.857 | 2.589 | 1.00 | 2.95 |
| ATOM | 202 | N | PRO | 14 | −3.671 | 6.053 | 2.539 | 1.00 | 3.66 |
| ATOM | 203 | CA | PRO | 14 | −4.438 | 6.341 | 1.318 | 1.00 | 4.09 |
| ATOM | 204 | HA | PRO | 14 | −5.448 | 5.975 | 1.401 | 1.00 | 4.39 |
| ATOM | 205 | CB | PRO | 14 | −4.454 | 7.870 | 1.263 | 1.00 | 5.04 |
| ATOM | 206 | HB1 | PRO | 14 | −5.342 | 8.242 | 1.750 | 1.00 | 5.62 |
| ATOM | 207 | HB2 | PRO | 14 | −4.437 | 8.197 | 0.234 | 1.00 | 5.18 |
| ATOM | 208 | CG | PRO | 14 | −3.224 | 8.289 | 1.986 | 1.00 | 5.32 |
| ATOM | 209 | HG1 | PRO | 14 | −3.368 | 9.268 | 2.418 | 1.00 | 5.91 |
| ATOM | 210 | HG2 | PRO | 14 | −2.384 | 8.296 | 1.308 | 1.00 | 5.77 |
| ATOM | 211 | CD | PRO | 14 | −3.010 | 7.267 | 3.068 | 1.00 | 4.46 |
| ATOM | 212 | HD1 | PRO | 14 | −3.474 | 7.590 | 3.989 | 1.00 | 4.68 |
| ATOM | 213 | HD2 | PRO | 14 | −1.956 | 7.102 | 3.216 | 1.00 | 4.53 |
| ATOM | 214 | C | PRO | 14 | −3.795 | 5.765 | 0.053 | 1.00 | 3.77 |
| ATOM | 215 | O | PRO | 14 | −2.821 | 5.013 | 0.129 | 1.00 | 4.07 |
| ATOM | 216 | N | CYS | 15 | −4.341 | 6.138 | −1.114 | 1.00 | 3.60 |
| ATOM | 217 | HN | CYS | 15 | −5.104 | 6.751 | −1.110 | 1.00 | 3.75 |
| ATOM | 218 | CA | CYS | 15 | −3.814 | 5.676 | −2.394 | 1.00 | 3.70 |
| ATOM | 219 | HA | CYS | 15 | −2.780 | 5.986 | −2.427 | 1.00 | 3.90 |
| ATOM | 220 | HB1 | CYS | 15 | −4.871 | 3.825 | −2.642 | 1.00 | 4.62 |
| ATOM | 221 | HB2 | CYS | 15 | −3.464 | 3.720 | −1.587 | 1.00 | 4.34 |
| ATOM | 222 | C | CYS | 15 | −4.533 | 6.333 | −3.587 | 1.00 | 3.85 |
| ATOM | 223 | O | CYS | 15 | −4.584 | 7.561 | −3.661 | 1.00 | 4.33 |
| ATOM | 224 | CB | CYS | 15 | −3.851 | 4.149 | −2.500 | 1.00 | 4.05 |
| ATOM | 225 | SG | CYS | 15 | −2.856 | 3.502 | −3.887 | 1.00 | 4.02 |
| ATOM | 226 | N | CYS | 16 | −5.057 | 5.523 | −4.536 | 1.00 | 3.70 |
| ATOM | 227 | HN | CYS | 16 | −4.985 | 4.556 | −4.441 | 1.00 | 3.50 |
| ATOM | 228 | CA | CYS | 16 | −5.710 | 6.043 | −5.736 | 1.00 | 4.08 |
| ATOM | 229 | HA | CYS | 16 | −5.636 | 5.275 | −6.496 | 1.00 | 3.93 |
| ATOM | 230 | HB1 | CYS | 16 | −7.294 | 7.293 | −4.999 | 1.00 | 4.46 |
| ATOM | 231 | HB2 | CYS | 16 | −7.597 | 5.564 | −4.810 | 1.00 | 4.09 |
| ATOM | 232 | C | CYS | 16 | −4.979 | 7.301 | −6.249 | 1.00 | 4.59 |
| ATOM | 233 | O | CYS | 16 | −5.610 | 8.273 | −6.672 | 1.00 | 5.05 |
| ATOM | 234 | CB | CYS | 16 | −7.196 | 6.327 | −5.471 | 1.00 | 4.36 |
| ATOM | 235 | SG | CYS | 16 | −8.216 | 6.341 | −6.984 | 1.00 | 5.13 |
| ATOM | 236 | N | ASP | 17 | −3.630 | 7.259 | −6.197 | 1.00 | 4.60 |
| ATOM | 237 | HN | ASP | 17 | −3.198 | 6.451 | −5.847 | 1.00 | 4.30 |
| ATOM | 238 | CA | ASP | 17 | −2.777 | 8.374 | −6.639 | 1.00 | 5.13 |
| ATOM | 239 | HA | ASP | 17 | −2.901 | 8.469 | −7.708 | 1.00 | 5.59 |
| ATOM | 240 | CB | ASP | 17 | −3.219 | 9.692 | −5.975 | 1.00 | 5.62 |
| ATOM | 241 | HB1 | ASP | 17 | −3.503 | 9.496 | −4.953 | 1.00 | 5.76 |
| ATOM | 242 | HB2 | ASP | 17 | −4.072 | 10.083 | −6.508 | 1.00 | 5.74 |
| ATOM | 243 | CG | ASP | 17 | −2.132 | 10.754 | −5.977 | 1.00 | 6.17 |
| ATOM | 244 | OD1 | ASP | 17 | −1.748 | 11.210 | −7.075 | 1.00 | 6.53 |
| ATOM | 245 | OD2 | ASP | 17 | −1.668 | 11.129 | −4.880 | 1.00 | 6.56 |
| ATOM | 246 | C | ASP | 17 | −1.283 | 8.093 | −6.351 | 1.00 | 4.80 |
| ATOM | 247 | O | ASP | 17 | −0.465 | 8.102 | −7.271 | 1.00 | 4.56 |
| ATOM | 248 | N | PRO | 18 | −0.908 | 7.839 | −5.070 | 1.00 | 5.13 |
| ATOM | 249 | CA | PRO | 18 | 0.485 | 7.553 | −4.669 | 1.00 | 5.06 |
| ATOM | 250 | HA | PRO | 18 | 1.165 | 8.301 | −5.023 | 1.00 | 5.33 |
| ATOM | 251 | CB | PRO | 18 | 0.438 | 7.600 | −3.127 | 1.00 | 5.85 |
| ATOM | 252 | HB1 | PRO | 18 | 1.243 | 8.221 | −2.761 | 1.00 | 6.32 |
| ATOM | 253 | HB2 | PRO | 18 | 0.542 | 6.601 | −2.729 | 1.00 | 5.79 |
| ATOM | 254 | CG | PRO | 18 | −0.891 | 8.182 | −2.786 | 1.00 | 6.39 |
| ATOM | 255 | HG1 | PRO | 18 | −0.814 | 9.257 | −2.712 | 1.00 | 6.94 |
| ATOM | 256 | HG2 | PRO | 18 | −1.249 | 7.765 | −1.857 | 1.00 | 6.75 |
| ATOM | 257 | CD | PRO | 18 | −1.800 | 7.803 | −3.912 | 1.00 | 5.92 |
| ATOM | 258 | HD1 | PRO | 18 | −2.598 | 8.519 | −4.012 | 1.00 | 6.35 |
| ATOM | 259 | HD2 | PRO | 18 | −2.194 | 6.810 | −3.762 | 1.00 | 5.99 |
| ATOM | 260 | C | PRO | 18 | 0.965 | 6.192 | −5.162 | 1.00 | 4.35 |
| ATOM | 261 | O | PRO | 18 | 0.484 | 5.690 | −6.181 | 1.00 | 4.45 |
| ATOM | 262 | N | CYS | 19 | 1.908 | 5.589 | −4.438 | 1.00 | 4.00 |
| ATOM | 263 | HN | CYS | 19 | 2.257 | 6.027 | −3.634 | 1.00 | 4.34 |
| ATOM | 264 | CA | CYS | 19 | 2.427 | 4.285 | −4.822 | 1.00 | 3.50 |
| ATOM | 265 | HA | CYS | 19 | 2.416 | 4.237 | −5.903 | 1.00 | 3.76 |
| ATOM | 266 | HB1 | CYS | 19 | 4.354 | 5.069 | −4.280 | 1.00 | 4.02 |
| ATOM | 267 | HB2 | CYS | 19 | 4.412 | 3.492 | −5.049 | 1.00 | 4.20 |
| ATOM | 268 | C | CYS | 19 | 1.501 | 3.201 | −4.285 | 1.00 | 2.98 |
| ATOM | 269 | O | CYS | 19 | 0.949 | 2.444 | −5.067 | 1.00 | 2.87 |
| ATOM | 270 | CB | CYS | 19 | 3.880 | 4.100 | −4.333 | 1.00 | 3.68 |
| ATOM | 271 | SG | CYS | 19 | 4.070 | 3.305 | −2.701 | 1.00 | 3.27 |
| ATOM | 272 | N | ALA | 20 | 1.301 | 3.192 | −2.958 | 1.00 | 3.00 |
| ATOM | 273 | HN | ALA | 20 | 1.746 | 3.891 | −2.427 | 1.00 | 3.43 |
| ATOM | 274 | CA | ALA | 20 | 0.381 | 2.259 | −2.261 | 1.00 | 2.68 |
| ATOM | 275 | HA | ALA | 20 | 0.867 | 1.965 | −1.352 | 1.00 | 2.43 |
| ATOM | 276 | CB | ALA | 20 | −0.896 | 2.998 | −1.882 | 1.00 | 2.88 |
| ATOM | 277 | HB1 | ALA | 20 | −1.747 | 2.351 | −2.033 | 1.00 | 3.03 |
| ATOM | 278 | HB2 | ALA | 20 | −0.997 | 3.878 | −2.499 | 1.00 | 3.21 |
| ATOM | 279 | HB3 | ALA | 20 | −0.847 | 3.293 | −0.841 | 1.00 | 3.09 |
| ATOM | 280 | C | ALA | 20 | 0.077 | 0.972 | −3.049 | 1.00 | 2.88 |
| ATOM | 281 | O | ALA | 20 | −0.665 | 1.040 | −3.996 | 1.00 | 3.63 |
| ATOM | 282 | N | THR | 21 | 0.639 | −0.179 | −2.606 | 1.00 | 2.52 |
| ATOM | 283 | HN | THR | 21 | 1.178 | −0.127 | −1.824 | 1.00 | 2.17 |
| ATOM | 284 | CA | THR | 21 | 0.492 | −1.519 | −3.247 | 1.00 | 3.01 |
| ATOM | 285 | HA | THR | 21 | 0.640 | −1.411 | −4.288 | 1.00 | 3.44 |
| ATOM | 286 | CB | THR | 21 | 1.615 | −2.430 | −2.712 | 1.00 | 3.51 |
| ATOM | 287 | HB | THR | 21 | 2.292 | −1.827 | −2.145 | 1.00 | 3.87 |
| ATOM | 288 | OG1 | THR | 21 | 2.342 | −3.018 | −3.765 | 1.00 | 3.92 |
| ATOM | 289 | HG1 | THR | 21 | 1.829 | −3.731 | −4.147 | 1.00 | 4.04 |
| ATOM | 290 | CG2 | THR | 21 | 1.139 | −3.534 | −1.814 | 1.00 | 3.64 |
| ATOM | 291 | HG21 | THR | 21 | 1.148 | −4.469 | −2.352 | 1.00 | 3.73 |
| ATOM | 292 | HG22 | THR | 21 | 0.138 | −3.302 | −1.513 | 1.00 | 3.73 |
| ATOM | 293 | HG23 | THR | 21 | 1.776 | −3.606 | −0.946 | 1.00 | 3.90 |
| ATOM | 294 | C | THR | 21 | −0.893 | −2.141 | −3.040 | 1.00 | 2.82 |
| ATOM | 295 | O | THR | 21 | −1.823 | −1.429 | −2.757 | 1.00 | 2.79 |
| ATOM | 296 | N | CYS | 22 | −1.047 | −3.453 | −3.225 | 1.00 | 3.23 |
| ATOM | 297 | HN | CYS | 22 | −0.284 | −3.987 | −3.488 | 1.00 | 3.75 |
| ATOM | 298 | CA | CYS | 22 | −2.361 | −4.091 | −3.092 | 1.00 | 3.29 |
| ATOM | 299 | HA | CYS | 22 | −2.941 | −3.546 | −2.350 | 1.00 | 2.73 |
| ATOM | 300 | HB1 | CYS | 22 | −2.576 | −4.621 | −5.160 | 1.00 | 4.32 |
| ATOM | 301 | HB2 | CYS | 22 | −3.066 | −2.975 | −4.783 | 1.00 | 4.25 |
| ATOM | 302 | C | CYS | 22 | −2.268 | −5.563 | −2.656 | 1.00 | 3.83 |
| ATOM | 303 | O | CYS | 22 | −2.573 | −6.463 | −3.444 | 1.00 | 4.63 |
| ATOM | 304 | CB | CYS | 22 | −3.092 | −3.999 | −4.442 | 1.00 | 3.86 |
| ATOM | 305 | SG | CYS | 22 | −4.841 | −4.522 | −4.418 | 1.00 | 3.79 |
| ATOM | 306 | N | TYR | 23 | −1.888 | −5.785 | −1.377 | 1.00 | 3.64 |
| ATOM | 307 | HN | TYR | 23 | −1.689 | −5.024 | −0.821 | 1.00 | 3.23 |
| ATOM | 308 | CA | TYR | 23 | −1.799 | −7.133 | −0.783 | 1.00 | 4.31 |
| ATOM | 309 | HA | TYR | 23 | −2.770 | −7.368 | −0.375 | 1.00 | 4.75 |
| ATOM | 310 | CB | TYR | 23 | −1.483 | −8.137 | −1.875 | 1.00 | 4.99 |
| ATOM | 311 | HB1 | TYR | 23 | −0.990 | −7.599 | −2.663 | 1.00 | 4.83 |
| ATOM | 312 | HB2 | TYR | 23 | −2.406 | −8.540 | −2.250 | 1.00 | 5.71 |
| ATOM | 313 | CG | TYR | 23 | −0.599 | −9.294 | −1.468 | 1.00 | 5.19 |
| ATOM | 314 | CD1 | TYR | 23 | −1.141 | −10.531 | −1.148 | 1.00 | 5.34 |
| ATOM | 315 | HD1 | TYR | 23 | −2.212 | −10.660 | −1.183 | 1.00 | 5.46 |
| ATOM | 316 | CD2 | TYR | 23 | 0.776 | −9.147 | −1.418 | 1.00 | 5.53 |
| ATOM | 317 | HD2 | TYR | 23 | 1.205 | −8.183 | −1.658 | 1.00 | 5.77 |
| ATOM | 318 | CE1 | TYR | 23 | −0.332 | −11.592 | −0.790 | 1.00 | 5.56 |
| ATOM | 319 | HE1 | TYR | 23 | −0.771 | −12.547 | −0.545 | 1.00 | 5.82 |
| ATOM | 320 | CE2 | TYR | 23 | 1.595 | −10.200 | −1.059 | 1.00 | 5.81 |
| ATOM | 321 | HE2 | TYR | 23 | 2.667 | −10.065 | −1.025 | 1.00 | 6.26 |
| ATOM | 322 | CZ | TYR | 23 | 1.036 | −11.421 | −0.748 | 1.00 | 5.71 |
| ATOM | 323 | OH | TYR | 23 | 1.846 | −12.475 | −0.395 | 1.00 | 6.01 |
| ATOM | 324 | HH | TYR | 23 | 2.160 | −12.917 | −1.187 | 1.00 | 6.05 |
| ATOM | 325 | C | TYR | 23 | −0.764 | −7.219 | 0.355 | 1.00 | 4.08 |
| ATOM | 326 | O | TYR | 23 | −0.942 | −7.990 | 1.300 | 1.00 | 4.31 |
| ATOM | 327 | N | CYS | 24 | 0.305 | −6.427 | 0.252 | 1.00 | 3.99 |
| ATOM | 328 | HN | CYS | 24 | 0.376 | −5.836 | −0.520 | 1.00 | 4.20 |
| ATOM | 329 | CA | CYS | 24 | 1.379 | −6.392 | 1.250 | 1.00 | 3.97 |
| ATOM | 330 | HA | CYS | 24 | 2.008 | −5.546 | 0.990 | 1.00 | 3.54 |
| ATOM | 331 | HB1 | CYS | 24 | 0.849 | −7.090 | 3.203 | 1.00 | 4.45 |
| ATOM | 332 | HB2 | CYS | 24 | −0.159 | −5.779 | 2.608 | 1.00 | 4.72 |
| ATOM | 333 | C | CYS | 24 | 2.230 | −7.664 | 1.271 | 1.00 | 4.56 |
| ATOM | 334 | O | CYS | 24 | 1.702 | −8.775 | 1.300 | 1.00 | 4.95 |
| ATOM | 335 | CB | CYS | 24 | 0.841 | −6.167 | 2.655 | 1.00 | 4.21 |
| ATOM | 336 | SG | CYS | 24 | 1.818 | −4.991 | 3.578 | 1.00 | 3.97 |
| ATOM | 337 | N | ARG | 25 | 3.553 | −7.485 | 1.306 | 1.00 | 4.81 |
| ATOM | 338 | HN | ARG | 25 | 3.910 | −6.572 | 1.315 | 1.00 | 4.70 |
| ATOM | 339 | CA | ARG | 25 | 4.485 | −8.614 | 1.370 | 1.00 | 5.41 |
| ATOM | 340 | HA | ARG | 25 | 4.148 | −9.364 | 0.676 | 1.00 | 5.78 |
| ATOM | 341 | CB | ARG | 25 | 5.903 | −8.174 | 0.986 | 1.00 | 5.35 |
| ATOM | 342 | HB1 | ARG | 25 | 6.586 | −8.982 | 1.206 | 1.00 | 5.48 |
| ATOM | 343 | HB2 | ARG | 25 | 6.173 | −7.317 | 1.584 | 1.00 | 5.55 |
| ATOM | 344 | CG | ARG | 25 | 6.065 | −7.802 | −0.478 | 1.00 | 5.19 |
| ATOM | 345 | HG1 | ARG | 25 | 5.093 | −7.787 | −0.945 | 1.00 | 5.37 |
| ATOM | 346 | HG2 | ARG | 25 | 6.687 | −8.541 | −0.961 | 1.00 | 5.36 |
| ATOM | 347 | CD | ARG | 25 | 6.712 | −6.433 | −0.636 | 1.00 | 5.12 |
| ATOM | 348 | HD1 | ARG | 25 | 6.606 | −5.890 | 0.292 | 1.00 | 5.22 |
| ATOM | 349 | HD2 | ARG | 25 | 6.201 | −5.899 | −1.423 | 1.00 | 4.97 |
| ATOM | 350 | NE | ARG | 25 | 8.134 | −6.527 | −0.973 | 1.00 | 5.60 |
| ATOM | 351 | HE | ARG | 25 | 8.367 | −6.596 | −1.923 | 1.00 | 5.89 |
| ATOM | 352 | CZ | ARG | 25 | 9.125 | −6.525 | −0.074 | 1.00 | 5.97 |
| ATOM | 353 | NH1 | ARG | 25 | 8.863 | −6.428 | 1.227 | 1.00 | 5.99 |
| ATOM | 354 | HH11 | ARG | 25 | 7.920 | −6.356 | 1.546 | 1.00 | 5.75 |
| ATOM | 355 | HH12 | ARG | 25 | 9.614 | −6.428 | 1.888 | 1.00 | 6.45 |
| ATOM | 356 | NH2 | ARG | 25 | 10.386 | −6.622 | −0.477 | 1.00 | 6.62 |
| ATOM | 357 | HH21 | ARG | 25 | 10.595 | −6.697 | −1.451 | 1.00 | 6.89 |
| ATOM | 358 | HH22 | ARG | 25 | 11.127 | −6.620 | 0.194 | 1.00 | 6.99 |
| ATOM | 359 | C | ARG | 25 | 4.514 | −9.217 | 2.773 | 1.00 | 5.75 |
| ATOM | 360 | O | ARG | 25 | 4.289 | −10.415 | 2.952 | 1.00 | 6.36 |
| ATOM | 361 | N | PHE | 26 | 4.802 | −8.369 | 3.759 | 1.00 | 5.44 |
| ATOM | 362 | HN | PHE | 26 | 4.967 | −7.433 | 3.534 | 1.00 | 4.97 |
| ATOM | 363 | CA | PHE | 26 | 4.879 | −8.793 | 5.158 | 1.00 | 5.87 |
| ATOM | 364 | HA | PHE | 26 | 4.990 | −9.862 | 5.151 | 1.00 | 6.37 |
| ATOM | 365 | CB | PHE | 26 | 6.121 | −8.187 | 5.838 | 1.00 | 5.87 |
| ATOM | 366 | HB1 | PHE | 26 | 6.993 | −8.742 | 5.519 | 1.00 | 6.14 |
| ATOM | 367 | HB2 | PHE | 26 | 6.026 | −8.279 | 6.907 | 1.00 | 5.89 |
| ATOM | 368 | CG | PHE | 26 | 6.361 | −6.740 | 5.515 | 1.00 | 5.68 |
| ATOM | 369 | CD1 | PHE | 26 | 6.033 | −5.748 | 6.420 | 1.00 | 5.91 |
| ATOM | 370 | HD1 | PHE | 26 | 5.595 | −6.022 | 7.368 | 1.00 | 6.30 |
| ATOM | 371 | CD2 | PHE | 26 | 6.920 | −6.381 | 4.303 | 1.00 | 5.59 |
| ATOM | 372 | HD2 | PHE | 26 | 7.172 | −7.156 | 3.593 | 1.00 | 5.76 |
| ATOM | 373 | CE1 | PHE | 26 | 6.259 | −4.419 | 6.122 | 1.00 | 5.89 |
| ATOM | 374 | HE1 | PHE | 26 | 5.999 | −3.653 | 6.837 | 1.00 | 6.28 |
| ATOM | 375 | CE2 | PHE | 26 | 7.150 | −5.054 | 3.995 | 1.00 | 5.57 |
| ATOM | 376 | HE2 | PHE | 26 | 7.587 | −4.787 | 3.044 | 1.00 | 5.74 |
| ATOM | 377 | CZ | PHE | 26 | 6.819 | −4.071 | 4.907 | 1.00 | 5.65 |
| ATOM | 378 | HZ | PHE | 26 | 6.997 | −3.032 | 4.670 | 1.00 | 5.75 |
| ATOM | 379 | C | PHE | 26 | 3.576 | −8.452 | 5.906 | 1.00 | 5.78 |
| ATOM | 380 | O | PHE | 26 | 2.491 | −8.694 | 5.373 | 1.00 | 5.69 |
| ATOM | 381 | N | PHE | 27 | 3.669 | −7.902 | 7.131 | 1.00 | 5.97 |
| ATOM | 382 | HN | PHE | 27 | 4.549 | −7.733 | 7.517 | 1.00 | 6.18 |
| ATOM | 383 | CA | PHE | 27 | 2.477 | −7.552 | 7.910 | 1.00 | 6.04 |
| ATOM | 384 | HA | PHE | 27 | 1.921 | −8.463 | 8.076 | 1.00 | 6.43 |
| ATOM | 385 | CB | PHE | 27 | 2.876 | −6.962 | 9.273 | 1.00 | 6.49 |
| ATOM | 386 | HB1 | PHE | 27 | 2.391 | −6.006 | 9.402 | 1.00 | 6.27 |
| ATOM | 387 | HB2 | PHE | 27 | 3.945 | −6.822 | 9.299 | 1.00 | 6.51 |
| ATOM | 388 | CG | PHE | 27 | 2.497 | −7.831 | 10.440 | 1.00 | 7.32 |
| ATOM | 389 | CD1 | PHE | 27 | 3.134 | −9.042 | 10.657 | 1.00 | 7.85 |
| ATOM | 390 | HD1 | PHE | 27 | 3.910 | −9.363 | 9.977 | 1.00 | 7.76 |
| ATOM | 391 | CD2 | PHE | 27 | 1.503 | −7.433 | 11.321 | 1.00 | 7.78 |
| ATOM | 392 | HD2 | PHE | 27 | 1.000 | −6.491 | 11.161 | 1.00 | 7.65 |
| ATOM | 393 | CE1 | PHE | 27 | 2.787 | −9.840 | 11.731 | 1.00 | 8.65 |
| ATOM | 394 | HE1 | PHE | 27 | 3.290 | −10.783 | 11.889 | 1.00 | 9.17 |
| ATOM | 395 | CE2 | PHE | 27 | 1.151 | −8.227 | 12.395 | 1.00 | 8.58 |
| ATOM | 396 | HE2 | PHE | 27 | 0.375 | −7.905 | 13.074 | 1.00 | 9.03 |
| ATOM | 397 | CZ | PHE | 27 | 1.794 | −9.432 | 12.601 | 1.00 | 8.94 |
| ATOM | 398 | HZ | PHE | 27 | 1.521 | −10.054 | 13.440 | 1.00 | 9.59 |
| ATOM | 399 | C | PHE | 27 | 1.587 | −6.578 | 7.130 | 1.00 | 5.30 |
| ATOM | 400 | O | PHE | 27 | 0.738 | −7.010 | 6.348 | 1.00 | 5.50 |
| ATOM | 401 | N | ASN | 28 | 1.790 | −5.270 | 7.320 | 1.00 | 4.68 |
| ATOM | 402 | HN | ASN | 28 | 2.489 | −4.973 | 7.933 | 1.00 | 4.87 |
| ATOM | 403 | CA | ASN | 28 | 1.011 | −4.272 | 6.600 | 1.00 | 3.97 |
| ATOM | 404 | HA | ASN | 28 | 0.924 | −4.630 | 5.591 | 1.00 | 3.66 |
| ATOM | 405 | CB | ASN | 28 | −0.399 | −4.127 | 7.180 | 1.00 | 4.05 |
| ATOM | 406 | HB1 | ASN | 28 | −0.734 | −3.109 | 7.042 | 1.00 | 4.27 |
| ATOM | 407 | HB2 | ASN | 28 | −0.372 | −4.353 | 8.235 | 1.00 | 4.30 |
| ATOM | 408 | CG | ASN | 28 | −1.411 | −5.060 | 6.513 | 1.00 | 4.05 |
| ATOM | 409 | OD1 | ASN | 28 | −2.267 | −5.631 | 7.187 | 1.00 | 4.69 |
| ATOM | 410 | ND2 | ASN | 28 | −1.320 | −5.227 | 5.185 | 1.00 | 3.69 |
| ATOM | 411 | HD21 | ASN | 28 | −0.620 | −4.749 | 4.698 | 1.00 | 3.34 |
| ATOM | 412 | HD22 | ASN | 28 | −1.961 | −5.829 | 4.749 | 1.00 | 4.01 |
| ATOM | 413 | C | ASN | 28 | 1.730 | −2.924 | 6.548 | 1.00 | 3.77 |
| ATOM | 414 | O | ASN | 28 | 1.560 | −2.065 | 7.416 | 1.00 | 3.98 |
| ATOM | 415 | N | ALA | 29 | 2.500 | −2.756 | 5.480 | 1.00 | 3.54 |
| ATOM | 416 | HN | ALA | 29 | 2.541 | −3.464 | 4.828 | 1.00 | 3.55 |
| ATOM | 417 | CA | ALA | 29 | 3.237 | −1.556 | 5.187 | 1.00 | 3.45 |
| ATOM | 418 | HA | ALA | 29 | 2.593 | −0.713 | 5.396 | 1.00 | 3.44 |
| ATOM | 419 | CB | ALA | 29 | 4.495 | −1.408 | 5.996 | 1.00 | 3.85 |
| ATOM | 420 | HB1 | ALA | 29 | 4.945 | −0.462 | 5.737 | 1.00 | 4.00 |
| ATOM | 421 | HB2 | ALA | 29 | 5.173 | −2.212 | 5.761 | 1.00 | 3.97 |
| ATOM | 422 | HB3 | ALA | 29 | 4.255 | −1.422 | 7.047 | 1.00 | 4.27 |
| ATOM | 423 | C | ALA | 29 | 3.563 | −1.564 | 3.712 | 1.00 | 3.33 |
| ATOM | 424 | O | ALA | 29 | 4.201 | −2.484 | 3.193 | 1.00 | 4.12 |
| ATOM | 425 | N | PHE | 30 | 3.068 | −0.553 | 3.063 | 1.00 | 2.67 |
| ATOM | 426 | HN | PHE | 30 | 2.558 | 0.083 | 3.580 | 1.00 | 2.45 |
| ATOM | 427 | CA | PHE | 30 | 3.209 | −0.378 | 1.613 | 1.00 | 2.74 |
| ATOM | 428 | HA | PHE | 30 | 2.894 | 0.626 | 1.371 | 1.00 | 2.83 |
| ATOM | 429 | CB | PHE | 30 | 4.669 | −0.566 | 1.171 | 1.00 | 3.35 |
| ATOM | 430 | HB1 | PHE | 30 | 4.796 | −0.143 | 0.185 | 1.00 | 3.67 |
| ATOM | 431 | HB2 | PHE | 30 | 4.891 | −1.622 | 1.133 | 1.00 | 3.59 |
| ATOM | 432 | CG | PHE | 30 | 5.673 | 0.083 | 2.088 | 1.00 | 3.86 |
| ATOM | 433 | CD1 | PHE | 30 | 5.441 | 1.346 | 2.615 | 1.00 | 4.35 |
| ATOM | 434 | HD1 | PHE | 30 | 4.529 | 1.867 | 2.361 | 1.00 | 4.45 |
| ATOM | 435 | CD2 | PHE | 30 | 6.847 | −0.573 | 2.424 | 1.00 | 4.38 |
| ATOM | 436 | HD2 | PHE | 30 | 7.040 | −1.557 | 2.020 | 1.00 | 4.49 |
| ATOM | 437 | CE1 | PHE | 30 | 6.360 | 1.938 | 3.459 | 1.00 | 5.11 |
| ATOM | 438 | HE1 | PHE | 30 | 6.167 | 2.922 | 3.862 | 1.00 | 5.69 |
| ATOM | 439 | CE2 | PHE | 30 | 7.769 | 0.016 | 3.266 | 1.00 | 5.15 |
| ATOM | 440 | HE2 | PHE | 30 | 8.681 | −0.505 | 3.520 | 1.00 | 5.77 |
| ATOM | 441 | CZ | PHE | 30 | 7.525 | 1.273 | 3.785 | 1.00 | 5.42 |
| ATOM | 442 | HZ | PHE | 30 | 8.245 | 1.735 | 4.444 | 1.00 | 6.14 |
| ATOM | 443 | C | PHE | 30 | 2.287 | −1.369 | 0.894 | 1.00 | 2.53 |
| ATOM | 444 | O | PHE | 30 | 2.704 | −2.085 | −0.011 | 1.00 | 3.13 |
| ATOM | 445 | N | CYS | 31 | 1.034 | −1.406 | 1.362 | 1.00 | 2.06 |
| ATOM | 446 | HN | CYS | 31 | 0.818 | −0.818 | 2.101 | 1.00 | 2.12 |
| ATOM | 447 | CA | CYS | 31 | −0.017 | −2.306 | 0.854 | 1.00 | 1.97 |
| ATOM | 448 | HA | CYS | 31 | 0.224 | −2.580 | −0.153 | 1.00 | 2.16 |
| ATOM | 449 | HB1 | CYS | 31 | 0.505 | −4.333 | 1.205 | 1.00 | 2.78 |
| ATOM | 450 | HB2 | CYS | 31 | −1.059 | −3.866 | 1.863 | 1.00 | 2.85 |
| ATOM | 451 | C | CYS | 31 | −1.410 | −1.651 | 0.917 | 1.00 | 1.56 |
| ATOM | 452 | O | CYS | 31 | −1.944 | −1.466 | 2.011 | 1.00 | 2.04 |
| ATOM | 453 | CB | CYS | 31 | −0.037 | −3.553 | 1.710 | 1.00 | 2.51 |
| ATOM | 454 | SG | CYS | 31 | 0.725 | −3.308 | 3.341 | 1.00 | 2.99 |
| ATOM | 455 | N | TYR | 32 | −1.997 | −1.284 | −0.236 | 1.00 | 1.35 |
| ATOM | 456 | HN | TYR | 32 | −1.539 | −1.446 | −1.075 | 1.00 | 1.77 |
| ATOM | 457 | CA | TYR | 32 | −3.327 | −0.635 | −0.238 | 1.00 | 1.25 |
| ATOM | 458 | HA | TYR | 32 | −3.903 | −1.138 | 0.503 | 1.00 | 1.48 |
| ATOM | 459 | CB | TYR | 32 | −3.228 | 0.842 | 0.167 | 1.00 | 1.68 |
| ATOM | 460 | HB1 | TYR | 32 | −4.003 | 1.065 | 0.885 | 1.00 | 1.99 |
| ATOM | 461 | HB2 | TYR | 32 | −3.365 | 1.461 | −0.707 | 1.00 | 2.16 |
| ATOM | 462 | CG | TYR | 32 | −1.909 | 1.201 | 0.781 | 1.00 | 2.00 |
| ATOM | 463 | CD1 | TYR | 32 | −0.740 | 1.028 | 0.066 | 1.00 | 2.67 |
| ATOM | 464 | HD1 | TYR | 32 | −0.802 | 0.652 | −0.942 | 1.00 | 3.10 |
| ATOM | 465 | CD2 | TYR | 32 | −1.829 | 1.678 | 2.069 | 1.00 | 2.45 |
| ATOM | 466 | HD2 | TYR | 32 | −2.743 | 1.831 | 2.638 | 1.00 | 2.72 |
| ATOM | 467 | CE1 | TYR | 32 | 0.484 | 1.317 | 0.605 | 1.00 | 3.29 |
| ATOM | 468 | HE1 | TYR | 32 | 1.382 | 1.166 | 0.001 | 1.00 | 3.99 |
| ATOM | 469 | CE2 | TYR | 32 | −0.612 | 1.976 | 2.629 | 1.00 | 3.14 |
| ATOM | 470 | HE2 | TYR | 32 | −0.576 | 2.335 | 3.635 | 1.00 | 3.79 |
| ATOM | 471 | CZ | TYR | 32 | 0.550 | 1.794 | 1.900 | 1.00 | 3.39 |
| ATOM | 472 | OH | TYR | 32 | 1.768 | 2.097 | 2.465 | 1.00 | 4.22 |
| ATOM | 473 | HH | TYR | 32 | 1.978 | 3.019 | 2.301 | 1.00 | 4.50 |
| ATOM | 474 | C | TYR | 32 | −4.105 | −0.726 | −1.553 | 1.00 | 1.55 |
| ATOM | 475 | O | TYR | 32 | −5.142 | −1.384 | −1.621 | 1.00 | 2.20 |
| ATOM | 476 | N | CYS | 33 | −3.660 | 0.019 | −2.571 | 1.00 | 2.04 |
| ATOM | 477 | HN | CYS | 33 | −2.872 | 0.584 | −2.442 | 1.00 | 2.31 |
| ATOM | 478 | CA | CYS | 33 | −4.394 | 0.077 | −3.831 | 1.00 | 2.83 |
| ATOM | 479 | HA | CYS | 33 | −5.173 | −0.658 | −3.776 | 1.00 | 3.09 |
| ATOM | 480 | HB1 | CYS | 33 | −5.053 | 1.928 | −2.929 | 1.00 | 3.26 |
| ATOM | 481 | HB2 | CYS | 33 | −6.070 | 1.345 | −4.241 | 1.00 | 3.20 |
| ATOM | 482 | C | CYS | 33 | −3.578 | −0.177 | −5.137 | 1.00 | 3.55 |
| ATOM | 483 | O | CYS | 33 | −4.167 | −0.120 | −6.219 | 1.00 | 4.43 |
| ATOM | 484 | CB | CYS | 33 | −5.055 | 1.457 | −3.915 | 1.00 | 3.01 |
| ATOM | 485 | SG | CYS | 33 | −4.215 | 2.580 | −5.069 | 1.00 | 3.40 |
| ATOM | 486 | N | ARG | 34 | −2.255 | −0.404 | −5.084 | 1.00 | 3.34 |
| ATOM | 487 | HN | ARG | 34 | −1.777 | −0.399 | −4.203 | 1.00 | 2.80 |
| ATOM | 488 | CA | ARG | 34 | −1.467 | −0.586 | −6.333 | 1.00 | 4.11 |
| ATOM | 489 | HA | ARG | 34 | −2.019 | −0.101 | −7.125 | 1.00 | 4.69 |
| ATOM | 490 | CB | ARG | 34 | −0.137 | 0.169 | −6.148 | 1.00 | 4.21 |
| ATOM | 491 | HB1 | ARG | 34 | 0.270 | −0.122 | −5.193 | 1.00 | 4.15 |
| ATOM | 492 | HB2 | ARG | 34 | −0.360 | 1.220 | −6.109 | 1.00 | 4.16 |
| ATOM | 493 | CG | ARG | 34 | 0.964 | −0.004 | −7.166 | 1.00 | 4.98 |
| ATOM | 494 | HG1 | ARG | 34 | 0.969 | 0.843 | −7.835 | 1.00 | 5.57 |
| ATOM | 495 | HG2 | ARG | 34 | 0.809 | −0.914 | −7.721 | 1.00 | 5.02 |
| ATOM | 496 | CD | ARG | 34 | 2.301 | −0.067 | −6.425 | 1.00 | 5.32 |
| ATOM | 497 | HD1 | ARG | 34 | 2.654 | −1.087 | −6.429 | 1.00 | 5.23 |
| ATOM | 498 | HD2 | ARG | 34 | 2.143 | 0.253 | −5.385 | 1.00 | 5.43 |
| ATOM | 499 | NE | ARG | 34 | 3.314 | 0.793 | −7.034 | 1.00 | 6.08 |
| ATOM | 500 | HE | ARG | 34 | 3.149 | 1.112 | −7.946 | 1.00 | 6.18 |
| ATOM | 501 | CZ | ARG | 34 | 4.441 | 1.162 | −6.420 | 1.00 | 6.88 |
| ATOM | 502 | NH1 | ARG | 34 | 4.701 | 0.747 | −5.183 | 1.00 | 7.12 |
| ATOM | 503 | HH11 | ARG | 34 | 4.052 | 0.155 | −4.705 | 1.00 | 6.71 |
| ATOM | 504 | HH12 | ARG | 34 | 5.546 | 1.028 | −4.728 | 1.00 | 7.87 |
| ATOM | 505 | NH2 | ARG | 34 | 5.313 | 1.944 | −7.048 | 1.00 | 7.69 |
| ATOM | 506 | HH21 | ARG | 34 | 5.125 | 2.257 | −7.979 | 1.00 | 7.76 |
| ATOM | 507 | HH22 | ARG | 34 | 6.157 | 2.222 | −6.589 | 1.00 | 8.36 |
| ATOM | 508 | C | ARG | 34 | −1.287 | −2.074 | −6.731 | 1.00 | 4.36 |
| ATOM | 509 | O | ARG | 34 | −2.259 | −2.732 | −7.106 | 1.00 | 4.73 |
| ATOM | 510 | N | LYS | 35 | −0.057 | −2.590 | −6.668 | 1.00 | 4.53 |
| ATOM | 511 | HN | LYS | 35 | 0.674 | −2.028 | −6.364 | 1.00 | 4.61 |
| ATOM | 512 | CA | LYS | 35 | 0.255 | −3.975 | −7.032 | 1.00 | 4.93 |
| ATOM | 513 | HA | LYS | 35 | −0.509 | 4.613 | −6.607 | 1.00 | 5.03 |
| ATOM | 514 | CB | LYS | 35 | 0.293 | −4.170 | −8.558 | 1.00 | 5.89 |
| ATOM | 515 | HB1 | LYS | 35 | −0.714 | −4.346 | −8.907 | 1.00 | 6.19 |
| ATOM | 516 | HB2 | LYS | 35 | 0.895 | −5.039 | −8.780 | 1.00 | 6.25 |
| ATOM | 517 | CG | LYS | 35 | 0.863 | −2.989 | −9.331 | 1.00 | 6.31 |
| ATOM | 518 | HG1 | LYS | 35 | 0.552 | −2.075 | −8.852 | 1.00 | 6.46 |
| ATOM | 519 | HG2 | LYS | 35 | 0.479 | −3.014 | −10.339 | 1.00 | 6.32 |
| ATOM | 520 | CD | LYS | 35 | 2.384 | −3.025 | −9.387 | 1.00 | 6.93 |
| ATOM | 521 | HD1 | LYS | 35 | 2.689 | −3.525 | −10.295 | 1.00 | 7.08 |
| ATOM | 522 | HD2 | LYS | 35 | 2.755 | −3.570 | −8.532 | 1.00 | 7.20 |
| ATOM | 523 | CE | LYS | 35 | 2.972 | −1.623 | −9.373 | 1.00 | 7.41 |
| ATOM | 524 | HE1 | LYS | 35 | 3.647 | −1.538 | −8.533 | 1.00 | 7.65 |
| ATOM | 525 | HE2 | LYS | 35 | 2.169 | −0.911 | −9.260 | 1.00 | 7.56 |
| ATOM | 526 | NZ | LYS | 35 | 3.716 | −1.314 | −10.626 | 1.00 | 7.75 |
| ATOM | 527 | HZ1 | LYS | 35 | 3.082 | −1.396 | −11.447 | 1.00 | 7.81 |
| ATOM | 528 | HZ2 | LYS | 35 | 4.509 | −1.975 | −10.746 | 1.00 | 8.01 |
| ATOM | 529 | HZ3 | LYS | 35 | 4.091 | −0.344 | −10.588 | 1.00 | 7.95 |
| ATOM | 530 | C | LYS | 35 | 1.596 | −4.335 | −6.418 | 1.00 | 4.53 |
| ATOM | 531 | O | LYS | 35 | 2.432 | −3.453 | −6.214 | 1.00 | 4.71 |
| ATOM | 532 | N | LEU | 36 | 1.805 | −5.602 | −6.086 | 1.00 | 4.30 |
| ATOM | 533 | HN | LEU | 36 | 1.130 | −6.257 | −6.243 | 1.00 | 4.47 |
| ATOM | 534 | CA | LEU | 36 | 3.036 | −5.989 | −5.453 | 1.00 | 4.13 |
| ATOM | 535 | HA | LEU | 36 | 3.354 | −5.105 | −4.916 | 1.00 | 3.77 |
| ATOM | 536 | CB | LEU | 36 | 2.843 | −7.055 | −4.416 | 1.00 | 4.20 |
| ATOM | 537 | HB1 | LEU | 36 | 3.786 | −7.540 | −4.232 | 1.00 | 4.50 |
| ATOM | 538 | HB2 | LEU | 36 | 2.120 | −7.776 | −4.770 | 1.00 | 4.66 |
| ATOM | 539 | CG | LEU | 36 | 2.360 | −6.422 | −3.135 | 1.00 | 3.78 |
| ATOM | 540 | HG | LEU | 36 | 2.622 | −5.367 | −3.189 | 1.00 | 3.57 |
| ATOM | 541 | CD1 | LEU | 36 | 0.849 | −6.524 | −3.055 | 1.00 | 4.07 |
| ATOM | 542 | HD11 | LEU | 36 | 0.535 | −6.468 | −2.028 | 1.00 | 4.35 |
| ATOM | 543 | HD12 | LEU | 36 | 0.528 | −7.464 | −3.481 | 1.00 | 4.26 |
| ATOM | 544 | HD13 | LEU | 36 | 0.405 | −5.712 | −3.610 | 1.00 | 4.30 |
| ATOM | 545 | CD2 | LEU | 36 | 3.040 | −7.016 | −1.907 | 1.00 | 4.09 |
| ATOM | 546 | HD21 | LEU | 36 | 4.020 | −6.576 | −1.793 | 1.00 | 4.26 |
| ATOM | 547 | HD22 | LEU | 36 | 3.138 | −8.084 | −2.027 | 1.00 | 4.48 |
| ATOM | 548 | HD23 | LEU | 36 | 2.447 | −6.802 | −1.026 | 1.00 | 4.28 |
| ATOM | 549 | C | LEU | 36 | 4.137 | −6.293 | −6.405 | 1.00 | 4.76 |
| ATOM | 550 | O | LEU | 36 | 4.441 | −7.429 | −6.775 | 1.00 | 5.31 |
| ATOM | 551 | N | GLY | 37 | 4.752 | −5.198 | −6.707 | 1.00 | 4.97 |
| ATOM | 552 | HN | GLY | 37 | 4.412 | −4.392 | −6.286 | 1.00 | 4.82 |
| ATOM | 553 | CA | GLY | 37 | 5.915 | −5.144 | −7.545 | 1.00 | 5.70 |
| ATOM | 554 | HA1 | GLY | 37 | 5.776 | −4.401 | −8.313 | 1.00 | 6.04 |
| ATOM | 555 | HA2 | GLY | 37 | 6.065 | −6.105 | −7.993 | 1.00 | 6.19 |
| ATOM | 556 | C | GLY | 37 | 7.120 | −4.776 | −6.704 | 1.00 | 5.54 |
| ATOM | 557 | O | GLY | 37 | 7.963 | −3.978 | −7.115 | 1.00 | 6.08 |
| ATOM | 558 | N | THR | 38 | 7.161 | −5.356 | −5.495 | 1.00 | 5.00 |
| ATOM | 559 | HN | THR | 38 | 6.446 | −5.959 | −5.236 | 1.00 | 4.79 |
| ATOM | 560 | CA | THR | 38 | 8.202 | −5.119 | −4.526 | 1.00 | 4.96 |
| ATOM | 561 | HA | THR | 38 | 8.007 | −5.786 | −3.699 | 1.00 | 5.16 |
| ATOM | 562 | CB | THR | 38 | 9.591 | −5.445 | −5.075 | 1.00 | 5.78 |
| ATOM | 563 | HB | THR | 38 | 10.154 | −4.531 | −5.184 | 1.00 | 6.05 |
| ATOM | 564 | OG1 | THR | 38 | 9.526 | −6.095 | −6.337 | 1.00 | 6.18 |
| ATOM | 565 | HG1 | THR | 38 | 9.079 | −6.941 | −6.244 | 1.00 | 6.46 |
| ATOM | 566 | CG2 | THR | 38 | 10.345 | −6.343 | −4.136 | 1.00 | 6.38 |
| ATOM | 567 | HG21 | THR | 38 | 9.756 | −7.232 | −3.961 | 1.00 | 6.46 |
| ATOM | 568 | HG22 | THR | 38 | 10.500 | −5.827 | −3.199 | 1.00 | 6.55 |
| ATOM | 569 | HG23 | THR | 38 | 11.296 | −6.614 | −4.570 | 1.00 | 6.89 |
| ATOM | 570 | C | THR | 38 | 8.149 | −3.700 | −3.987 | 1.00 | 4.17 |
| ATOM | 571 | O | THR | 38 | 8.747 | −2.781 | −4.553 | 1.00 | 4.31 |
| ATOM | 572 | N | ALA | 39 | 7.460 | −3.542 | −2.852 | 1.00 | 3.68 |
| ATOM | 573 | HN | ALA | 39 | 7.044 | −4.326 | −2.437 | 1.00 | 3.92 |
| ATOM | 574 | CA | ALA | 39 | 7.370 | −2.242 | −2.190 | 1.00 | 3.19 |
| ATOM | 575 | HA | ALA | 39 | 6.953 | −1.533 | −2.893 | 1.00 | 3.39 |
| ATOM | 576 | CB | ALA | 39 | 6.459 | −2.305 | −0.952 | 1.00 | 2.97 |
| ATOM | 577 | HB1 | ALA | 39 | 5.453 | −1.989 | −1.214 | 1.00 | 3.17 |
| ATOM | 578 | HB2 | ALA | 39 | 6.845 | −1.652 | −0.184 | 1.00 | 3.05 |
| ATOM | 579 | HB3 | ALA | 39 | 6.429 | −3.317 | −0.578 | 1.00 | 3.21 |
| ATOM | 580 | C | ALA | 39 | 8.789 | −1.809 | −1.820 | 1.00 | 3.22 |
| ATOM | 581 | O | ALA | 39 | 9.116 | −0.625 | −1.846 | 1.00 | 3.39 |
| ATOM | 582 | N | MET | 40 | 9.628 | −2.825 | −1.539 | 1.00 | 3.43 |
| ATOM | 583 | HN | MET | 40 | 9.270 | −3.743 | −1.595 | 1.00 | 3.65 |
| ATOM | 584 | CA | MET | 40 | 11.056 | −2.668 | −1.215 | 1.00 | 3.68 |
| ATOM | 585 | HA | MET | 40 | 11.340 | −3.533 | −0.635 | 1.00 | 3.98 |
| ATOM | 586 | CB | MET | 40 | 11.888 | −2.677 | −2.505 | 1.00 | 4.33 |
| ATOM | 587 | HB1 | MET | 40 | 12.568 | −1.837 | −2.491 | 1.00 | 4.41 |
| ATOM | 588 | HB2 | MET | 40 | 11.222 | −2.575 | −3.350 | 1.00 | 4.46 |
| ATOM | 589 | CG | MET | 40 | 12.704 | −3.945 | −2.692 | 1.00 | 5.16 |
| ATOM | 590 | HG1 | MET | 40 | 12.841 | −4.118 | −3.751 | 1.00 | 5.45 |
| ATOM | 591 | HG2 | MET | 40 | 12.162 | −4.774 | −2.262 | 1.00 | 5.31 |
| ATOM | 592 | SD | MET | 40 | 14.325 | −3.847 | −1.907 | 1.00 | 6.00 |
| ATOM | 593 | CE | MET | 40 | 15.309 | −3.173 | −3.244 | 1.00 | 6.60 |
| ATOM | 594 | HE1 | MET | 40 | 15.124 | −3.736 | −4.146 | 1.00 | 6.92 |
| ATOM | 595 | HE2 | MET | 40 | 16.356 | −3.239 | −2.987 | 1.00 | 6.56 |
| ATOM | 596 | HE3 | MET | 40 | 15.041 | −2.139 | −3.404 | 1.00 | 7.01 |
| ATOM | 597 | C | MET | 40 | 11.386 | −1.423 | −0.381 | 1.00 | 3.37 |
| ATOM | 598 | O | MET | 40 | 11.559 | −1.513 | 0.835 | 1.00 | 3.68 |
| ATOM | 599 | N | ASN | 41 | 11.510 | −0.272 | −1.048 | 1.00 | 3.35 |
| ATOM | 600 | HN | ASN | 41 | 11.386 | −0.269 | −2.021 | 1.00 | 3.59 |
| ATOM | 601 | CA | ASN | 41 | 11.864 | 0.977 | −0.377 | 1.00 | 3.57 |
| ATOM | 602 | HA | ASN | 41 | 12.586 | 0.745 | 0.390 | 1.00 | 4.02 |
| ATOM | 603 | CB | ASN | 41 | 12.503 | 1.948 | −1.377 | 1.00 | 4.36 |
| ATOM | 604 | HB1 | ASN | 41 | 12.506 | 2.941 | −0.952 | 1.00 | 4.61 |
| ATOM | 605 | HB2 | ASN | 41 | 11.920 | 1.953 | −2.286 | 1.00 | 4.37 |
| ATOM | 606 | CG | ASN | 41 | 13.932 | 1.569 | −1.727 | 1.00 | 5.30 |
| ATOM | 607 | OD1 | ASN | 41 | 14.191 | 0.473 | −2.226 | 1.00 | 5.84 |
| ATOM | 608 | ND2 | ASN | 41 | 14.868 | 2.477 | −1.470 | 1.00 | 5.81 |
| ATOM | 609 | HD21 | ASN | 41 | 14.592 | 3.331 | −1.074 | 1.00 | 5.65 |
| ATOM | 610 | HD22 | ASN | 41 | 15.798 | 2.256 | −1.685 | 1.00 | 6.50 |
| ATOM | 611 | C | ASN | 41 | 10.651 | 1.636 | 0.277 | 1.00 | 3.02 |
| ATOM | 612 | O | ASN | 41 | 9.507 | 1.390 | −0.108 | 1.00 | 3.02 |
| ATOM | 613 | N | PRO | 42 | 10.898 | 2.490 | 1.291 | 1.00 | 3.12 |
| ATOM | 614 | CA | PRO | 42 | 9.833 | 3.192 | 2.018 | 1.00 | 3.15 |
| ATOM | 615 | HA | PRO | 42 | 9.154 | 2.496 | 2.484 | 1.00 | 3.54 |
| ATOM | 616 | CB | PRO | 42 | 10.588 | 3.974 | 3.102 | 1.00 | 4.06 |
| ATOM | 617 | HB1 | PRO | 42 | 10.553 | 3.428 | 4.033 | 1.00 | 4.67 |
| ATOM | 618 | HB2 | PRO | 42 | 10.133 | 4.945 | 3.230 | 1.00 | 4.13 |
| ATOM | 619 | CG | PRO | 42 | 11.983 | 4.089 | 2.593 | 1.00 | 4.46 |
| ATOM | 620 | HG1 | PRO | 42 | 12.673 | 4.160 | 3.421 | 1.00 | 5.17 |
| ATOM | 621 | HG2 | PRO | 42 | 12.072 | 4.956 | 1.954 | 1.00 | 4.81 |
| ATOM | 622 | CD | PRO | 42 | 12.235 | 2.834 | 1.810 | 1.00 | 3.83 |
| ATOM | 623 | HD1 | PRO | 42 | 12.611 | 2.055 | 2.456 | 1.00 | 4.33 |
| ATOM | 624 | HD2 | PRO | 42 | 12.927 | 3.025 | 1.002 | 1.00 | 3.84 |
| ATOM | 625 | C | PRO | 42 | 9.043 | 4.145 | 1.125 | 1.00 | 2.55 |
| ATOM | 626 | O | PRO | 42 | 9.610 | 5.065 | 0.531 | 1.00 | 2.68 |
| ATOM | 627 | N | CYS | 43 | 7.731 | 3.923 | 1.041 | 1.00 | 2.61 |
| ATOM | 628 | HN | CYS | 43 | 7.340 | 3.178 | 1.541 | 1.00 | 3.08 |
| ATOM | 629 | CA | CYS | 43 | 6.862 | 4.768 | 0.230 | 1.00 | 2.66 |
| ATOM | 630 | HA | CYS | 43 | 7.382 | 5.025 | −0.623 | 1.00 | 3.10 |
| ATOM | 631 | HB1 | CYS | 43 | 4.942 | 3.935 | 0.709 | 1.00 | 2.52 |
| ATOM | 632 | HB2 | CYS | 43 | 5.840 | 3.031 | −0.503 | 1.00 | 2.27 |
| ATOM | 633 | C | CYS | 43 | 6.535 | 6.042 | 0.962 | 1.00 | 3.31 |
| ATOM | 634 | O | CYS | 43 | 6.796 | 7.152 | 0.494 | 1.00 | 3.75 |
| ATOM | 635 | CB | CYS | 43 | 5.579 | 4.021 | −0.159 | 1.00 | 2.41 |
| ATOM | 636 | SG | CYS | 43 | 4.616 | 4.831 | −1.478 | 1.00 | 3.14 |
| ATOM | 637 | N | SER | 44 | 5.994 | 5.829 | 2.121 | 1.00 | 3.89 |
| ATOM | 638 | HN | SER | 44 | 5.876 | 4.902 | 2.362 | 1.00 | 3.89 |
| ATOM | 639 | CA | SER | 44 | 5.605 | 6.891 | 3.055 | 1.00 | 4.87 |
| ATOM | 640 | HA | SER | 44 | 4.903 | 6.474 | 3.757 | 1.00 | 5.17 |
| ATOM | 641 | CB | SER | 44 | 6.842 | 7.366 | 3.832 | 1.00 | 5.33 |
| ATOM | 642 | HB1 | SER | 44 | 7.327 | 6.513 | 4.284 | 1.00 | 5.78 |
| ATOM | 643 | HB2 | SER | 44 | 6.539 | 8.054 | 4.606 | 1.00 | 5.38 |
| ATOM | 644 | OG | SER | 44 | 7.771 | 8.016 | 2.981 | 1.00 | 5.71 |
| ATOM | 645 | HG | SER | 44 | 8.433 | 7.384 | 2.689 | 1.00 | 5.91 |
| ATOM | 646 | C | SER | 44 | 4.916 | 8.060 | 2.338 | 1.00 | 5.60 |
| ATOM | 647 | O | SER | 44 | 4.324 | 7.887 | 1.270 | 1.00 | 5.82 |
| ATOM | 648 | N | ARG | 45 | 4.992 | 9.244 | 2.946 | 1.00 | 6.31 |
| ATOM | 649 | HN | ARG | 45 | 5.466 | 9.302 | 3.798 | 1.00 | 6.38 |
| ATOM | 650 | CA | ARG | 45 | 4.385 | 10.456 | 2.392 | 1.00 | 7.28 |
| ATOM | 651 | HA | ARG | 45 | 4.713 | 11.288 | 2.997 | 1.00 | 7.53 |
| ATOM | 652 | CB | ARG | 45 | 4.845 | 10.690 | 0.944 | 1.00 | 7.96 |
| ATOM | 653 | HB1 | ARG | 45 | 4.706 | 11.733 | 0.701 | 1.00 | 8.31 |
| ATOM | 654 | HB2 | ARG | 45 | 4.231 | 10.094 | 0.285 | 1.00 | 7.94 |
| ATOM | 655 | CG | ARG | 45 | 6.301 | 10.331 | 0.684 | 1.00 | 8.60 |
| ATOM | 656 | HG1 | ARG | 45 | 6.471 | 10.321 | −0.383 | 1.00 | 8.78 |
| ATOM | 657 | HG2 | ARG | 45 | 6.498 | 9.351 | 1.089 | 1.00 | 8.86 |
| ATOM | 658 | CD | ARG | 45 | 7.254 | 11.328 | 1.326 | 1.00 | 9.04 |
| ATOM | 659 | HD1 | ARG | 45 | 6.712 | 11.904 | 2.062 | 1.00 | 9.20 |
| ATOM | 660 | HD2 | ARG | 45 | 7.632 | 11.990 | 0.560 | 1.00 | 8.91 |
| ATOM | 661 | NE | ARG | 45 | 8.384 | 10.664 | 1.981 | 1.00 | 9.74 |
| ATOM | 662 | HE | ARG | 45 | 8.404 | 10.675 | 2.960 | 1.00 | 9.93 |
| ATOM | 663 | CZ | ARG | 45 | 9.375 | 10.047 | 1.328 | 1.00 | 10.31 |
| ATOM | 664 | NH1 | ARG | 45 | 9.388 | 10.005 | −0.002 | 1.00 | 10.31 |
| ATOM | 665 | HH11 | ARG | 45 | 8.653 | 10.435 | −0.525 | 1.00 | 9.86 |
| ATOM | 666 | HH12 | ARG | 45 | 10.134 | 9.541 | −0.479 | 1.00 | 10.88 |
| ATOM | 667 | NH2 | ARG | 45 | 10.357 | 9.466 | 2.011 | 1.00 | 11.06 |
| ATOM | 668 | HH21 | ARG | 45 | 10.355 | 9.490 | 3.011 | 1.00 | 11.21 |
| ATOM | 669 | H22 | ARG | 45 | 11.100 | 9.004 | 1.525 | 1.00 | 11.58 |
| ATOM | 670 | C | ARG | 45 | 2.858 | 10.387 | 2.457 | 1.00 | 7.69 |
| ATOM | 671 | O | ARG | 45 | 2.168 | 10.651 | 1.468 | 1.00 | 8.34 |
| ATOM | 672 | N | THR | 46 | 2.336 | 10.036 | 3.633 | 1.00 | 7.60 |
| ATOM | 673 | HN | THR | 46 | 2.935 | 9.842 | 4.379 | 1.00 | 7.26 |
| ATOM | 674 | CA | THR | 46 | 0.895 | 9.934 | 3.836 | 1.00 | 8.32 |
| ATOM | 675 | HA | THR | 46 | 0.488 | 9.396 | 2.997 | 1.00 | 8.41 |
| ATOM | 676 | CB | THR | 46 | 0.582 | 9.157 | 5.122 | 1.00 | 8.61 |
| ATOM | 677 | HB | THR | 46 | −0.480 | 9.219 | 5.314 | 1.00 | 8.70 |
| ATOM | 678 | OG1 | THR | 46 | 1.266 | 9.717 | 6.233 | 1.00 | 8.93 |
| ATOM | 679 | HG1 | THR | 46 | 2.175 | 9.410 | 6.243 | 1.00 | 9.05 |
| ATOM | 680 | CG2 | THR | 46 | 0.951 | 7.688 | 5.037 | 1.00 | 8.89 |
| ATOM | 681 | HG21 | THR | 46 | 0.433 | 7.232 | 4.206 | 1.00 | 9.07 |
| ATOM | 682 | HG22 | THR | 46 | 0.668 | 7.191 | 5.954 | 1.00 | 9.06 |
| ATOM | 683 | HG23 | THR | 46 | 2.017 | 7.593 | 4.891 | 1.00 | 9.01 |
| ATOM | 684 | C | THR | 46 | 0.249 | 11.319 | 3.889 | 1.00 | 9.05 |
| ATOM | 685 | OT1 | THR | 46 | 0.759 | 12.187 | 4.632 | 1.00 | 9.34 |
| ATOM | 686 | OT2 | THR | 46 | −0.761 | 11.525 | 3.184 | 1.00 | 9.53 |

TABLE 5
MARP atomic coordinates in 3 dimensional
space determine by NMR at 800 mHz.

| | | | | | | | | | | | |
|---|---|---|---|---|---|---|---|---|---|---|---|
| ATOM | 1 | N | CYS | A | 1 | −12.726 | −3.631 | 3.103 | 1.00 | 0.00 | N |
| ATOM | 2 | CA | CYS | A | 1 | −11.311 | −3.175 | 3.172 | 1.00 | 0.00 | C |
| ATOM | 3 | C | CYS | A | 1 | −10.813 | −2.734 | 1.797 | 1.00 | 0.00 | C |
| ATOM | 4 | O | CYS | A | 1 | −11.608 | −2.465 | 0.895 | 1.00 | 0.00 | O |
| ATOM | 5 | CB | CYS | A | 1 | −10.421 | −4.311 | 3.710 | 1.00 | 0.00 | C |
| ATOM | 6 | SG | CYS | A | 1 | −11.306 | −5.794 | 4.301 | 1.00 | 0.00 | S |
| ATOM | 7 | H | CYS | A | 1 | −13.283 | −2.870 | 2.668 | 1.00 | 0.00 | H |
| ATOM | 8 | HA | CYS | A | 1 | −11.258 | −2.333 | 3.848 | 1.00 | 0.00 | H |
| ATOM | 9 | 1HB | CYS | A | 1 | −9.754 | −4.626 | 2.925 | 1.00 | 0.00 | H |
| ATOM | 10 | 2HB | CYS | A | 1 | −9.837 | −3.932 | 4.534 | 1.00 | 0.00 | H |
| ATOM | 11 | N | VAL | A | 2 | −9.494 | −2.666 | 1.644 | 1.00 | 0.00 | N |
| ATOM | 12 | CA | VAL | A | 2 | −8.887 | −2.267 | 0.382 | 1.00 | 0.00 | C |
| ATOM | 13 | C | VAL | A | 2 | −8.548 | −3.483 | −0.461 | 1.00 | 0.00 | C |
| ATOM | 14 | O | VAL | A | 2 | −8.532 | −4.609 | 0.036 | 1.00 | 0.00 | O |
| ATOM | 15 | CB | VAL | A | 2 | −7.610 | −1.429 | 0.600 | 1.00 | 0.00 | C |
| ATOM | 16 | CG1 | VAL | A | 2 | −7.264 | −0.642 | −0.655 | 1.00 | 0.00 | C |
| ATOM | 17 | CG2 | VAL | A | 2 | −7.772 | −0.495 | 1.792 | 1.00 | 0.00 | C |
| ATOM | 18 | H | VAL | A | 2 | −8.913 | −2.896 | 2.399 | 1.00 | 0.00 | H |
| ATOM | 19 | HA | VAL | A | 2 | −9.602 | −1.668 | −0.161 | 1.00 | 0.00 | H |
| ATOM | 20 | HB | VAL | A | 2 | −6.794 | −2.106 | 0.807 | 1.00 | 0.00 | H |
| ATOM | 21 | 1HG1 | VAL | A | 2 | −6.616 | 0.182 | −0.397 | 1.00 | 0.00 | H |
| ATOM | 22 | 2HG1 | VAL | A | 2 | −8.171 | −0.259 | −1.100 | 1.00 | 0.00 | H |
| ATOM | 23 | 3HG1 | VAL | A | 2 | −6.763 | −1.289 | −1.359 | 1.00 | 0.00 | H |
| ATOM | 24 | 1HG2 | VAL | A | 2 | −8.466 | 0.293 | 1.540 | 1.00 | 0.00 | H |
| ATOM | 25 | 2HG2 | VAL | A | 2 | −6.814 | −0.065 | 2.043 | 1.00 | 0.00 | H |
| ATOM | 26 | 3HG2 | VAL | A | 2 | −8.150 | −1.052 | 2.637 | 1.00 | 0.00 | H |
| ATOM | 27 | N | ARG | A | 3 | −8.292 | −3.256 | −1.740 | 1.00 | 0.00 | N |
| ATOM | 28 | CA | ARG | A | 3 | −7.971 | −4.344 | −2.645 | 1.00 | 0.00 | C |
| ATOM | 29 | C | ARG | A | 3 | −6.520 | −4.785 | −2.479 | 1.00 | 0.00 | C |
| ATOM | 30 | O | ARG | A | 3 | −5.866 | −4.443 | −1.495 | 1.00 | 0.00 | O |
| ATOM | 31 | CB | ARG | A | 3 | −8.238 | −3.931 | −4.093 | 1.00 | 0.00 | C |
| ATOM | 32 | CG | ARG | A | 3 | −9.563 | −3.209 | −4.286 | 1.00 | 0.00 | C |
| ATOM | 33 | CD | ARG | A | 3 | −10.155 | −3.490 | −5.657 | 1.00 | 0.00 | C |
| ATOM | 34 | NE | ARG | A | 3 | −11.422 | −2.790 | −5.860 | 1.00 | 0.00 | N |
| ATOM | 35 | CZ | ARG | A | 3 | −11.970 | −2.581 | −7.054 | 1.00 | 0.00 | C |
| ATOM | 36 | NH1 | ARG | A | 3 | −11.368 | −3.017 | −8.154 | 1.00 | 0.00 | N |
| ATOM | 37 | NH2 | ARG | A | 3 | −13.123 | −1.934 | −7.151 | 1.00 | 0.00 | N |
| ATOM | 38 | H | ARG | A | 3 | −8.331 | −2.339 | −2.086 | 1.00 | 0.00 | H |
| ATOM | 39 | HA | ARG | A | 3 | −8.617 | −5.168 | −2.389 | 1.00 | 0.00 | H |
| ATOM | 40 | 1HB | ARG | A | 3 | −7.445 | −3.274 | −4.421 | 1.00 | 0.00 | H |
| ATOM | 41 | 2HB | ARG | A | 3 | −8.241 | −4.815 | −4.714 | 1.00 | 0.00 | H |
| ATOM | 42 | 1HG | ARG | A | 3 | −10.256 | −3.545 | −3.529 | 1.00 | 0.00 | H |
| ATOM | 43 | 2HG | ARG | A | 3 | −9.401 | −2.148 | −4.183 | 1.00 | 0.00 | H |
| ATOM | 44 | 1HD | ARG | A | 3 | −9.454 | −3.168 | −6.411 | 1.00 | 0.00 | H |
| ATOM | 45 | 2HD | ARG | A | 3 | −10.323 | −4.553 | −5.752 | 1.00 | 0.00 | H |
| ATOM | 46 | HE | ARG | A | 3 | −11.888 | −2.458 | −5.064 | 1.00 | 0.00 | H |
| ATOM | 47 | 1HH1 | ARG | A | 3 | −10.497 | −3.506 | −8.088 | 1.00 | 0.00 | H |
| ATOM | 48 | 2HH1 | ARG | A | 3 | −11.784 | −2.858 | −9.049 | 1.00 | 0.00 | H |
| ATOM | 49 | 1HH2 | ARG | A | 3 | −13.582 | −1.605 | −6.326 | 1.00 | 0.00 | H |
| ATOM | 50 | 2HH2 | ARG | A | 3 | −13.534 | −1.777 | −8.049 | 1.00 | 0.00 | H |
| ATOM | 51 | N | LEU | A | 4 | −6.023 | −5.552 | −3.445 | 1.00 | 0.00 | N |
| ATOM | 52 | CA | LEU | A | 4 | −4.657 | −6.049 | −3.403 | 1.00 | 0.00 | C |
| ATOM | 53 | C | LEU | A | 4 | −3.655 | −4.912 | −3.579 | 1.00 | 0.00 | C |
| ATOM | 54 | O | LEU | A | 4 | −2.627 | −4.869 | −2.904 | 1.00 | 0.00 | O |
| ATOM | 55 | CB | LEU | A | 4 | −4.453 | −7.095 | −4.498 | 1.00 | 0.00 | C |
| ATOM | 56 | CG | LEU | A | 4 | −3.030 | −7.636 | −4.611 | 1.00 | 0.00 | C |
| ATOM | 57 | CD1 | LEU | A | 4 | −2.800 | −8.754 | −3.607 | 1.00 | 0.00 | C |
| ATOM | 58 | CD2 | LEU | A | 4 | −2.756 | −8.121 | −6.027 | 1.00 | 0.00 | C |
| ATOM | 59 | H | LEU | A | 4 | −6.593 | −5.796 | −4.201 | 1.00 | 0.00 | H |
| ATOM | 60 | HA | LEU | A | 4 | −4.498 | −6.509 | −2.441 | 1.00 | 0.00 | H |
| ATOM | 61 | 1HB | LEU | A | 4 | −5.119 | −7.921 | −4.303 | 1.00 | 0.00 | H |
| ATOM | 62 | 2HB | LEU | A | 4 | −4.722 | −6.653 | −5.444 | 1.00 | 0.00 | H |
| ATOM | 63 | HG | LEU | A | 4 | −2.339 | −6.840 | −4.390 | 1.00 | 0.00 | H |
| ATOM | 64 | 1HD1 | LEU | A | 4 | −1.768 | −9.067 | −3.646 | 1.00 | 0.00 | H |
| ATOM | 65 | 2HD1 | LEU | A | 4 | −3.439 | −9.590 | −3.847 | 1.00 | 0.00 | H |
| ATOM | 66 | 3HD1 | LEU | A | 4 | −3.031 | −8.398 | −2.613 | 1.00 | 0.00 | H |
| ATOM | 67 | 1HD2 | LEU | A | 4 | −3.092 | −9.142 | −6.129 | 1.00 | 0.00 | H |
| ATOM | 68 | 2HD2 | LEU | A | 4 | −1.694 | −8.068 | −6.226 | 1.00 | 0.00 | H |
| ATOM | 69 | 3HD2 | LEU | A | 4 | −3.284 | −7.495 | −6.731 | 1.00 | 0.00 | H |
| ATOM | 70 | N | HIS | A | 5 | −3.963 | −3.995 | −4.487 | 1.00 | 0.00 | N |
| ATOM | 71 | CA | HIS | A | 5 | −3.096 | −2.863 | −4.747 | 1.00 | 0.00 | C |
| ATOM | 72 | C | HIS | A | 5 | −3.884 | −1.696 | −5.334 | 1.00 | 0.00 | C |
| ATOM | 73 | O | HIS | A | 5 | −3.679 | −1.308 | −6.484 | 1.00 | 0.00 | O |
| ATOM | 74 | CB | HIS | A | 5 | −1.958 | −3.263 | −5.689 | 1.00 | 0.00 | C |
| ATOM | 75 | CG | HIS | A | 5 | −2.420 | −4.027 | −6.891 | 1.00 | 0.00 | C |
| ATOM | 76 | ND1 | HIS | A | 5 | −1.567 | −4.754 | −7.694 | 1.00 | 0.00 | N |
| ATOM | 77 | CD2 | HIS | A | 5 | −3.655 | −4.175 | −7.426 | 1.00 | 0.00 | C |
| ATOM | 78 | CE1 | HIS | A | 5 | −2.255 | −5.317 | −8.670 | 1.00 | 0.00 | C |
| ATOM | 79 | NE2 | HIS | A | 5 | −3.524 | −4.982 | −8.530 | 1.00 | 0.00 | N |
| ATOM | 80 | H | HIS | A | 5 | −4.790 | −4.078 | −4.985 | 1.00 | 0.00 | H |
| ATOM | 81 | HA | HIS | A | 5 | −2.685 | −2.559 | −3.808 | 1.00 | 0.00 | H |
| ATOM | 82 | 1HB | HIS | A | 5 | −1.457 | −2.372 | −6.034 | 1.00 | 0.00 | H |
| ATOM | 83 | 2HB | HIS | A | 5 | −1.256 | −3.881 | −5.149 | 1.00 | 0.00 | H |
| ATOM | 84 | HD1 | HIS | A | 5 | −0.598 | −4.844 | −7.567 | 1.00 | 0.00 | H |
| ATOM | 85 | HD2 | HIS | A | 5 | −4.572 | −3.740 | −7.054 | 1.00 | 0.00 | H |
| ATOM | 86 | HE1 | HIS | A | 5 | −1.850 | −5.946 | −9.449 | 1.00 | 0.00 | H |
| ATOM | 87 | HE2 | HIS | A | 5 | −4.265 | −5.332 | −9.068 | 1.00 | 0.00 | H |
| ATOM | 88 | N | GLU | A | 6 | −4.784 | −1.141 | −4.532 | 1.00 | 0.00 | N |
| ATOM | 89 | CA | GLU | A | 6 | −5.605 | −0.015 | −4.963 | 1.00 | 0.00 | C |
| ATOM | 90 | C | GLU | A | 6 | −5.739 | 1.011 | −3.845 | 1.00 | 0.00 | C |
| ATOM | 91 | O | GLU | A | 6 | −5.364 | 0.752 | −2.701 | 1.00 | 0.00 | O |
| ATOM | 92 | CB | GLU | A | 6 | −6.988 | −0.503 | −5.397 | 1.00 | 0.00 | C |
| ATOM | 93 | CG | GLU | A | 6 | −7.710 | 0.464 | −6.322 | 1.00 | 0.00 | C |
| ATOM | 94 | CD | GLU | A | 6 | −8.816 | −0.205 | −7.114 | 1.00 | 0.00 | C |
| ATOM | 95 | OE1 | GLU | A | 6 | −8.635 | −1.372 | −7.517 | 1.00 | 0.00 | O |
| ATOM | 96 | OE2 | GLU | A | 6 | −9.863 | 0.441 | −7.332 | 1.00 | 0.00 | O |
| ATOM | 97 | H | GLU | A | 6 | −4.899 | −1.494 | −3.625 | 1.00 | 0.00 | H |
| ATOM | 98 | HA | GLU | A | 6 | −5.113 | 0.449 | −5.804 | 1.00 | 0.00 | H |
| ATOM | 99 | 1HB | GLU | A | 6 | −6.879 | −1.445 | −5.911 | 1.00 | 0.00 | H |
| ATOM | 100 | 2HB | GLU | A | 6 | −7.597 | −0.651 | −4.518 | 1.00 | 0.00 | H |
| ATOM | 101 | 1HG | GLU | A | 6 | −8.142 | 1.256 | −5.727 | 1.00 | 0.00 | H |
| ATOM | 102 | 2HG | GLU | A | 6 | −6.993 | 0.884 | −7.013 | 1.00 | 0.00 | H |
| ATOM | 103 | N | SER | A | 7 | −6.278 | 2.179 | −4.180 | 1.00 | 0.00 | N |
| ATOM | 104 | CA | SER | A | 7 | −6.462 | 3.245 | −3.202 | 1.00 | 0.00 | C |
| ATOM | 105 | C | SER | A | 7 | −7.396 | 2.797 | −2.083 | 1.00 | 0.00 | C |
| ATOM | 106 | O | SER | A | 7 | −8.345 | 2.047 | −2.316 | 1.00 | 0.00 | O |
| ATOM | 107 | CB | SER | A | 7 | −7.018 | 4.497 | −3.880 | 1.00 | 0.00 | C |
| ATOM | 108 | OG | SER | A | 7 | −6.070 | 5.060 | −4.769 | 1.00 | 0.00 | O |
| ATOM | 109 | H | SER | A | 7 | −6.559 | 2.327 | −5.108 | 1.00 | 0.00 | H |
| ATOM | 110 | HA | SER | A | 7 | −5.496 | 3.473 | −2.778 | 1.00 | 0.00 | H |
| ATOM | 111 | 1HB | SER | A | 7 | −7.905 | 4.237 | −4.439 | 1.00 | 0.00 | H |
| ATOM | 112 | 2HB | SER | A | 7 | −7.269 | 5.230 | −3.128 | 1.00 | 0.00 | H |
| ATOM | 113 | HG | SER | A | 7 | −6.144 | 4.637 | −5.629 | 1.00 | 0.00 | H |
| ATOM | 114 | N | CYS | A | 8 | −7.124 | 3.260 | −0.867 | 1.00 | 0.00 | N |
| ATOM | 115 | CA | CYS | A | 8 | −7.941 | 2.904 | 0.286 | 1.00 | 0.00 | C |
| ATOM | 116 | C | CYS | A | 8 | −8.771 | 4.094 | 0.762 | 1.00 | 0.00 | C |
| ATOM | 117 | O | CYS | A | 8 | −9.807 | 3.916 | 1.401 | 1.00 | 0.00 | O |
| ATOM | 118 | CB | CYS | A | 8 | −7.058 | 2.390 | 1.427 | 1.00 | 0.00 | C |
| ATOM | 119 | SG | CYS | A | 8 | −5.978 | 3.656 | 2.170 | 1.00 | 0.00 | S |
| ATOM | 120 | H | CYS | A | 8 | −6.353 | 3.853 | −0.744 | 1.00 | 0.00 | H |
| ATOM | 121 | HA | CYS | A | 8 | −8.611 | 2.117 | −0.017 | 1.00 | 0.00 | H |
| ATOM | 122 | 1HB | CYS | A | 8 | −7.688 | 1.998 | 2.211 | 1.00 | 0.00 | H |
| ATOM | 123 | 2HB | CYS | A | 8 | −6.426 | 1.599 | 1.051 | 1.00 | 0.00 | H |
| ATOM | 124 | N | LEU | A | 9 | −8.302 | 5.303 | 0.439 | 1.00 | 0.00 | N |
| ATOM | 125 | CA | LEU | A | 9 | −8.981 | 6.552 | 0.818 | 1.00 | 0.00 | C |
| ATOM | 126 | C | LEU | A | 9 | −9.886 | 6.374 | 2.037 | 1.00 | 0.00 | C |
| ATOM | 127 | O | LEU | A | 9 | −11.044 | 5.977 | 1.911 | 1.00 | 0.00 | O |
| ATOM | 128 | CB | LEU | A | 9 | −9.802 | 7.079 | −0.360 | 1.00 | 0.00 | C |
| ATOM | 129 | CG | LEU | A | 9 | −10.634 | 6.025 | −1.093 | 1.00 | 0.00 | C |
| ATOM | 130 | CD1 | LEU | A | 9 | −11.893 | 6.651 | −1.675 | 1.00 | 0.00 | C |
| ATOM | 131 | CD2 | LEU | A | 9 | −9.811 | 5.362 | −2.186 | 1.00 | 0.00 | C |
| ATOM | 132 | H | LEU | A | 9 | −7.471 | 5.361 | −0.072 | 1.00 | 0.00 | H |
| ATOM | 133 | HA | LEU | A | 9 | −8.219 | 7.277 | 1.059 | 1.00 | 0.00 | H |
| ATOM | 134 | 1HB | LEU | A | 9 | −10.469 | 7.845 | 0.009 | 1.00 | 0.00 | H |
| ATOM | 135 | 2HB | LEU | A | 9 | −9.124 | 7.528 | −1.071 | 1.00 | 0.00 | H |
| ATOM | 136 | HG | LEU | A | 9 | −10.935 | 5.263 | −0.391 | 1.00 | 0.00 | H |
| ATOM | 137 | 1HD1 | LEU | A | 9 | −11.708 | 7.693 | −1.894 | 1.00 | 0.00 | H |
| ATOM | 138 | 2HD1 | LEU | A | 9 | −12.698 | 6.571 | −0.961 | 1.00 | 0.00 | H |
| ATOM | 139 | 3HD1 | LEU | A | 9 | −12.164 | 6.134 | −2.583 | 1.00 | 0.00 | H |
| ATOM | 140 | 1HD2 | LEU | A | 9 | −8.792 | 5.249 | −1.850 | 1.00 | 0.00 | H |
| ATOM | 141 | 2HD2 | LEU | A | 9 | −9.830 | 5.978 | −3.074 | 1.00 | 0.00 | H |
| ATOM | 142 | 3HD2 | LEU | A | 9 | −10.227 | 4.393 | −2.413 | 1.00 | 0.00 | H |
| ATOM | 143 | N | GLY | A | 10 | −9.348 | 6.673 | 3.215 | 1.00 | 0.00 | N |
| ATOM | 144 | CA | GLY | A | 10 | −10.121 | 6.541 | 4.436 | 1.00 | 0.00 | C |
| ATOM | 145 | C | GLY | A | 10 | −9.254 | 6.529 | 5.682 | 1.00 | 0.00 | C |
| ATOM | 146 | O | GLY | A | 10 | −9.723 | 6.856 | 6.773 | 1.00 | 0.00 | O |
| ATOM | 147 | H | GLY | A | 10 | −8.422 | 6.989 | 3.254 | 1.00 | 0.00 | H |
| ATOM | 148 | 1HA | GLY | A | 10 | −10.812 | 7.369 | 4.502 | 1.00 | 0.00 | H |
| ATOM | 149 | 2HA | GLY | A | 10 | −10.685 | 5.622 | 4.393 | 1.00 | 0.00 | H |
| ATOM | 150 | N | GLN | A | 11 | −7.987 | 6.152 | 5.523 | 1.00 | 0.00 | N |
| ATOM | 151 | CA | GLN | A | 11 | −7.049 | 6.098 | 6.646 | 1.00 | 0.00 | C |
| ATOM | 152 | C | GLN | A | 11 | −7.329 | 4.901 | 7.553 | 1.00 | 0.00 | C |
| ATOM | 153 | O | GLN | A | 11 | −6.435 | 4.097 | 7.819 | 1.00 | 0.00 | O |
| ATOM | 154 | CB | GLN | A | 11 | −7.109 | 7.391 | 7.462 | 1.00 | 0.00 | C |
| ATOM | 155 | CG | GLN | A | 11 | −7.285 | 8.643 | 6.616 | 1.00 | 0.00 | C |
| ATOM | 156 | CD | GLN | A | 11 | −6.672 | 9.870 | 7.259 | 1.00 | 0.00 | C |
| ATOM | 157 | OE1 | GLN | A | 11 | −5.574 | 9.813 | 7.813 | 1.00 | 0.00 | O |
| ATOM | 158 | NE2 | GLN | A | 11 | −7.382 | 10.990 | 7.193 | 1.00 | 0.00 | N |
| ATOM | 159 | H | GLN | A | 11 | −7.672 | 5.903 | 4.630 | 1.00 | 0.00 | H |
| ATOM | 160 | HA | GLN | A | 11 | −6.059 | 5.995 | 6.238 | 1.00 | 0.00 | H |
| ATOM | 161 | 1HB | GLN | A | 11 | −7.936 | 7.326 | 8.146 | 1.00 | 0.00 | H |
| ATOM | 162 | 2HB | GLN | A | 11 | −6.193 | 7.488 | 8.025 | 1.00 | 0.00 | H |
| ATOM | 163 | 1HG | GLN | A | 11 | −6.813 | 8.483 | 5.658 | 1.00 | 0.00 | H |
| ATOM | 164 | 2HG | GLN | A | 11 | −8.341 | 8.817 | 6.471 | 1.00 | 0.00 | H |
| ATOM | 165 | 1HE2 | GLN | A | 11 | −8.249 | 10.961 | 6.738 | 1.00 | 0.00 | H |
| ATOM | 166 | 2HE2 | GLN | A | 11 | −7.010 | 11.799 | 7.601 | 1.00 | 0.00 | H |
| ATOM | 167 | N | GLN | A | 12 | −8.564 | 4.785 | 8.029 | 1.00 | 0.00 | N |
| ATOM | 168 | CA | GLN | A | 12 | −8.941 | 3.684 | 8.907 | 1.00 | 0.00 | C |
| ATOM | 169 | C | GLN | A | 12 | −9.394 | 2.459 | 8.111 | 1.00 | 0.00 | C |
| ATOM | 170 | O | GLN | A | 12 | −9.943 | 1.514 | 8.677 | 1.00 | 0.00 | O |
| ATOM | 171 | CB | GLN | A | 12 | −10.056 | 4.121 | 9.865 | 1.00 | 0.00 | C |
| ATOM | 172 | CG | GLN | A | 12 | −11.051 | 5.103 | 9.266 | 1.00 | 0.00 | C |
| ATOM | 173 | CD | GLN | A | 12 | −11.598 | 4.640 | 7.930 | 1.00 | 0.00 | C |
| ATOM | 174 | OE1 | GLN | A | 12 | −10.903 | 4.677 | 6.915 | 1.00 | 0.00 | O |
| ATOM | 175 | NE2 | GLN | A | 12 | −12.851 | 4.199 | 7.924 | 1.00 | 0.00 | N |
| ATOM | 176 | H | GLN | A | 12 | −9.236 | 5.452 | 7.790 | 1.00 | 0.00 | H |
| ATOM | 177 | HA | GLN | A | 12 | −8.072 | 3.415 | 9.485 | 1.00 | 0.00 | H |
| ATOM | 178 | 1HB | GLN | A | 12 | −10.599 | 3.248 | 10.177 | 1.00 | 0.00 | H |
| ATOM | 179 | 2HB | GLN | A | 12 | −9.606 | 4.582 | 10.733 | 1.00 | 0.00 | H |
| ATOM | 180 | 1HG | GLN | A | 12 | −11.877 | 5.223 | 9.952 | 1.00 | 0.00 | H |
| ATOM | 181 | 2HG | GLN | A | 12 | −10.559 | 6.055 | 9.126 | 1.00 | 0.00 | H |
| ATOM | 182 | 1HE2 | GLN | A | 12 | −13.347 | 4.199 | 8.771 | 1.00 | 0.00 | H |
| ATOM | 183 | 2HE2 | GLN | A | 12 | −13.231 | 3.892 | 7.074 | 1.00 | 0.00 | H |
| ATOM | 184 | N | VAL | A | 13 | −9.160 | 2.473 | 6.800 | 1.00 | 0.00 | N |
| ATOM | 185 | CA | VAL | A | 13 | −9.542 | 1.354 | 5.946 | 1.00 | 0.00 | C |
| ATOM | 186 | C | VAL | A | 13 | −8.322 | 0.528 | 5.546 | 1.00 | 0.00 | C |
| ATOM | 187 | O | VAL | A | 13 | −7.729 | 0.753 | 4.492 | 1.00 | 0.00 | O |
| ATOM | 188 | CB | VAL | A | 13 | −10.263 | 1.836 | 4.669 | 1.00 | 0.00 | C |
| ATOM | 189 | CG1 | VAL | A | 13 | −10.752 | 0.652 | 3.847 | 1.00 | 0.00 | C |
| ATOM | 190 | CG2 | VAL | A | 13 | −11.419 | 2.758 | 5.027 | 1.00 | 0.00 | C |
| ATOM | 191 | H | VAL | A | 13 | −8.718 | 3.248 | 6.399 | 1.00 | 0.00 | H |
| ATOM | 192 | HA | VAL | A | 13 | −10.221 | 0.725 | 6.502 | 1.00 | 0.00 | H |
| ATOM | 193 | HB | VAL | A | 13 | −9.557 | 2.393 | 4.070 | 1.00 | 0.00 | H |
| ATOM | 194 | 1HG1 | VAL | A | 13 | −11.816 | 0.529 | 3.990 | 1.00 | 0.00 | H |
| ATOM | 195 | 2HG1 | VAL | A | 13 | −10.241 | −0.244 | 4.164 | 1.00 | 0.00 | H |
| ATOM | 196 | 3HG1 | VAL | A | 13 | −10.548 | 0.831 | 2.801 | 1.00 | 0.00 | H |
| ATOM | 197 | 1HG2 | VAL | A | 13 | −11.116 | 3.785 | 4.886 | 1.00 | 0.00 | H |
| ATOM | 198 | 2HG2 | VAL | A | 13 | −11.696 | 2.604 | 6.059 | 1.00 | 0.00 | H |
| ATOM | 199 | 3HG2 | VAL | A | 13 | −12.264 | 2.543 | 4.391 | 1.00 | 0.00 | H |
| ATOM | 200 | N | PRO | A | 14 | −7.930 | −0.446 | 6.386 | 1.00 | 0.00 | N |
| ATOM | 201 | CA | PRO | A | 14 | −6.777 | −1.305 | 6.110 | 1.00 | 0.00 | C |
| ATOM | 202 | C | PRO | A | 14 | −7.034 | −2.258 | 4.950 | 1.00 | 0.00 | C |
| ATOM | 203 | O | PRO | A | 14 | −8.181 | −2.502 | 4.578 | 1.00 | 0.00 | O |
| ATOM | 204 | CB | PRO | A | 14 | −6.591 | −2.086 | 7.411 | 1.00 | 0.00 | C |
| ATOM | 205 | CG | PRO | A | 14 | −7.935 | −2.085 | 8.053 | 1.00 | 0.00 | C |
| ATOM | 206 | CD | PRO | A | 14 | −8.582 | −0.784 | 7.665 | 1.00 | 0.00 | C |
| ATOM | 207 | HA | PRO | A | 14 | −5.889 | −0.723 | 5.908 | 1.00 | 0.00 | H |
| ATOM | 208 | 1HB | PRO | A | 14 | −6.261 | −3.090 | 7.187 | 1.00 | 0.00 | H |
| ATOM | 209 | 2HB | PRO | A | 14 | −5.859 | −1.592 | 8.030 | 1.00 | 0.00 | H |
| ATOM | 210 | 1HG | PRO | A | 14 | −8.518 | −2.917 | 7.685 | 1.00 | 0.00 | H |
| ATOM | 211 | 2HG | PRO | A | 14 | −7.831 | −2.146 | 9.126 | 1.00 | 0.00 | H |
| ATOM | 212 | 1HD | PRO | A | 14 | −9.646 | −0.916 | 7.532 | 1.00 | 0.00 | H |
| ATOM | 213 | 2HD | PRO | A | 14 | −8.384 | −0.027 | 8.409 | 1.00 | 0.00 | H |
| ATOM | 214 | N | CYS | A | 15 | −5.961 | −2.791 | 4.378 | 1.00 | 0.00 | N |
| ATOM | 215 | CA | CYS | A | 15 | −6.075 | −3.717 | 3.259 | 1.00 | 0.00 | C |
| ATOM | 216 | C | CYS | A | 15 | −6.799 | −4.995 | 3.672 | 1.00 | 0.00 | C |
| ATOM | 217 | O | CYS | A | 15 | −6.585 | −5.518 | 4.766 | 1.00 | 0.00 | O |
| ATOM | 218 | CB | CYS | A | 15 | −4.694 | −4.058 | 2.701 | 1.00 | 0.00 | C |
| ATOM | 219 | SG | CYS | A | 15 | −4.736 | −4.924 | 1.101 | 1.00 | 0.00 | S |
| ATOM | 220 | H | CYS | A | 15 | −5.072 | −2.557 | 4.720 | 1.00 | 0.00 | H |
| ATOM | 221 | HA | CYS | A | 15 | −6.652 | −3.229 | 2.487 | 1.00 | 0.00 | H |
| ATOM | 222 | 1HB | CYS | A | 15 | −4.133 | −3.147 | 2.567 | 1.00 | 0.00 | H |
| ATOM | 223 | 2HB | CYS | A | 15 | −4.177 | −4.692 | 3.407 | 1.00 | 0.00 | H |
| ATOM | 224 | N | CYS | A | 16 | −7.654 | −5.495 | 2.785 | 1.00 | 0.00 | N |
| ATOM | 225 | CA | CYS | A | 16 | −8.412 | −6.714 | 3.043 | 1.00 | 0.00 | C |
| ATOM | 226 | C | CYS | A | 16 | −7.515 | −7.955 | 2.994 | 1.00 | 0.00 | C |
| ATOM | 227 | O | CYS | A | 16 | −7.967 | −9.063 | 3.287 | 1.00 | 0.00 | O |
| ATOM | 228 | CB | CYS | A | 16 | −9.544 | −6.852 | 2.023 | 1.00 | 0.00 | C |
| ATOM | 229 | SG | CYS | A | 16 | −11.200 | −7.104 | 2.750 | 1.00 | 0.00 | S |
| ATOM | 230 | H | CYS | A | 16 | −7.774 | −5.030 | 1.929 | 1.00 | 0.00 | H |
| ATOM | 231 | HA | CYS | A | 16 | −8.838 | −6.639 | 4.025 | 1.00 | 0.00 | H |
| ATOM | 232 | 1HB | CYS | A | 16 | −9.585 | −5.961 | 1.417 | 1.00 | 0.00 | H |
| ATOM | 233 | 2HB | CYS | A | 16 | −9.334 | −7.698 | 1.391 | 1.00 | 0.00 | H |
| ATOM | 234 | N | ASP | A | 17 | −6.252 | −7.772 | 2.614 | 1.00 | 0.00 | N |
| ATOM | 235 | CA | ASP | A | 17 | −5.316 | −8.888 | 2.521 | 1.00 | 0.00 | C |
| ATOM | 236 | C | ASP | A | 17 | −4.478 | −9.026 | 3.793 | 1.00 | 0.00 | C |
| ATOM | 237 | O | ASP | A | 17 | −4.012 | −8.032 | 4.356 | 1.00 | 0.00 | O |
| ATOM | 238 | CB | ASP | A | 17 | −4.398 | −8.705 | 1.311 | 1.00 | 0.00 | C |
| ATOM | 239 | CG | ASP | A | 17 | −5.169 | −8.577 | 0.014 | 1.00 | 0.00 | C |
| ATOM | 240 | OD1 | ASP | A | 17 | −6.127 | −7.776 | −0.030 | 1.00 | 0.00 | O |
| ATOM | 241 | OD2 | ASP | A | 17 | −4.818 | −9.278 | −0.959 | 1.00 | 0.00 | O |
| ATOM | 242 | H | ASP | A | 17 | −5.944 | −6.872 | 2.383 | 1.00 | 0.00 | H |
| ATOM | 243 | HA | ASP | A | 17 | −5.895 | −9.789 | 2.384 | 1.00 | 0.00 | H |
| ATOM | 244 | 1HB | ASP | A | 17 | −3.807 | −7.811 | 1.448 | 1.00 | 0.00 | H |
| ATOM | 245 | 2HB | ASP | A | 17 | −3.739 | −9.559 | 1.236 | 1.00 | 0.00 | H |
| ATOM | 246 | N | PRO | A | 18 | −4.267 | −10.271 | 4.261 | 1.00 | 0.00 | N |
| ATOM | 247 | CA | PRO | A | 18 | −3.475 | −10.536 | 5.463 | 1.00 | 0.00 | C |
| ATOM | 248 | C | PRO | A | 18 | −2.086 | −9.917 | 5.375 | 1.00 | 0.00 | C |
| ATOM | 249 | O | PRO | A | 18 | −1.417 | −10.011 | 4.345 | 1.00 | 0.00 | O |
| ATOM | 250 | CB | PRO | A | 18 | −3.381 | −12.068 | 5.519 | 1.00 | 0.00 | C |
| ATOM | 251 | CG | PRO | A | 18 | −3.806 | −12.542 | 4.170 | 1.00 | 0.00 | C |
| ATOM | 252 | CD | PRO | A | 18 | −4.767 | −11.513 | 3.654 | 1.00 | 0.00 | C |
| ATOM | 253 | HA | PRO | A | 18 | −3.973 | −10.170 | 6.350 | 1.00 | 0.00 | H |
| ATOM | 254 | 1HB | PRO | A | 18 | −2.363 | −12.360 | 5.737 | 1.00 | 0.00 | H |
| ATOM | 255 | 2HB | PRO | A | 18 | −4.038 | −12.441 | 6.291 | 1.00 | 0.00 | H |
| ATOM | 256 | 1HG | PRO | A | 18 | −2.947 | −12.613 | 3.519 | 1.00 | 0.00 | H |
| ATOM | 257 | 2HG | PRO | A | 18 | −4.293 | −13.501 | 4.254 | 1.00 | 0.00 | H |
| ATOM | 258 | 1HD | PRO | A | 18 | −4.726 | −11.462 | 2.575 | 1.00 | 0.00 | H |
| ATOM | 259 | 2HD | PRO | A | 18 | −5.770 | −11.730 | 3.988 | 1.00 | 0.00 | H |
| ATOM | 260 | N | CYS | A | 19 | −1.661 | −9.276 | 6.456 | 1.00 | 0.00 | N |
| ATOM | 261 | CA | CYS | A | 19 | −0.355 | −8.631 | 6.501 | 1.00 | 0.00 | C |
| ATOM | 262 | C | CYS | A | 19 | −0.270 | −7.482 | 5.495 | 1.00 | 0.00 | C |
| ATOM | 263 | O | CYS | A | 19 | 0.816 | −6.977 | 5.211 | 1.00 | 0.00 | O |
| ATOM | 264 | CB | CYS | A | 19 | 0.753 | −9.652 | 6.222 | 1.00 | 0.00 | C |
| ATOM | 265 | SG | CYS | A | 19 | 2.439 | −9.011 | 6.483 | 1.00 | 0.00 | S |
| ATOM | 266 | H | CYS | A | 19 | −2.242 | −9.230 | 7.243 | 1.00 | 0.00 | H |
| ATOM | 267 | HA | CYS | A | 19 | −0.218 | −8.232 | 7.495 | 1.00 | 0.00 | H |
| ATOM | 268 | 1HB | CYS | A | 19 | 0.620 | −10.502 | 6.875 | 1.00 | 0.00 | H |
| ATOM | 269 | 2HB | CYS | A | 19 | 0.679 | −9.980 | 5.195 | 1.00 | 0.00 | H |
| ATOM | 270 | N | ALA | A | 20 | −1.418 | −7.065 | 4.959 | 1.00 | 0.00 | N |
| ATOM | 271 | CA | ALA | A | 20 | −1.450 | −5.971 | 3.995 | 1.00 | 0.00 | C |
| ATOM | 272 | C | ALA | A | 20 | −2.007 | −4.701 | 4.631 | 1.00 | 0.00 | C |
| ATOM | 273 | O | ALA | A | 20 | −3.025 | −4.738 | 5.322 | 1.00 | 0.00 | O |
| ATOM | 274 | CB | ALA | A | 20 | −2.271 | −6.363 | 2.777 | 1.00 | 0.00 | C |
| ATOM | 275 | H | ALA | A | 20 | −2.259 | −7.496 | 5.218 | 1.00 | 0.00 | H |
| ATOM | 276 | HA | ALA | A | 20 | −0.435 | −5.784 | 3.671 | 1.00 | 0.00 | H |
| ATOM | 277 | 1HB | ALA | A | 20 | −3.318 | −6.393 | 3.041 | 1.00 | 0.00 | H |
| ATOM | 278 | 2HB | ALA | A | 20 | −1.960 | −7.339 | 2.431 | 1.00 | 0.00 | H |
| ATOM | 279 | 3HB | ALA | A | 20 | −2.119 | −5.638 | 1.992 | 1.00 | 0.00 | H |
| ATOM | 280 | N | THR | A | 21 | −1.332 | −3.580 | 4.395 | 1.00 | 0.00 | N |
| ATOM | 281 | CA | THR | A | 21 | −1.761 | −2.300 | 4.948 | 1.00 | 0.00 | C |
| ATOM | 282 | C | THR | A | 21 | −1.668 | −1.197 | 3.900 | 1.00 | 0.00 | C |
| ATOM | 283 | O | THR | A | 21 | −0.790 | −1.223 | 3.038 | 1.00 | 0.00 | O |
| ATOM | 284 | CB | THR | A | 21 | −0.909 | −1.938 | 6.166 | 1.00 | 0.00 | C |
| ATOM | 285 | OG1 | THR | A | 21 | −1.401 | −0.766 | 6.792 | 1.00 | 0.00 | O |
| ATOM | 286 | CG2 | THR | A | 21 | 0.547 | −1.702 | 5.830 | 1.00 | 0.00 | C |
| ATOM | 287 | H | THR | A | 21 | −0.526 | −3.615 | 3.837 | 1.00 | 0.00 | H |
| ATOM | 288 | HA | THR | A | 21 | −2.791 | −2.400 | 5.257 | 1.00 | 0.00 | H |
| ATOM | 289 | HB | THR | A | 21 | −0.957 | −2.749 | 6.879 | 1.00 | 0.00 | H |
| ATOM | 290 | HG1 | THR | A | 21 | −1.514 | −0.074 | 6.135 | 1.00 | 0.00 | H |
| ATOM | 291 | 1HG2 | THR | A | 21 | 0.824 | −2.313 | 4.984 | 1.00 | 0.00 | H |
| ATOM | 292 | 2HG2 | THR | A | 21 | 1.160 | −1.965 | 6.680 | 1.00 | 0.00 | H |
| ATOM | 293 | 3HG2 | THR | A | 21 | 0.697 | −0.660 | 5.588 | 1.00 | 0.00 | H |
| ATOM | 294 | N | CYS | A | 22 | −2.578 | −0.231 | 3.975 | 1.00 | 0.00 | N |
| ATOM | 295 | CA | CYS | A | 22 | −2.593 | 0.876 | 3.026 | 1.00 | 0.00 | C |
| ATOM | 296 | C | CYS | A | 22 | −1.412 | 1.813 | 3.253 | 1.00 | 0.00 | C |
| ATOM | 297 | O | CYS | A | 22 | −1.294 | 2.439 | 4.306 | 1.00 | 0.00 | O |
| ATOM | 298 | CB | CYS | A | 22 | −3.901 | 1.658 | 3.132 | 1.00 | 0.00 | C |
| ATOM | 299 | SG | CYS | A | 22 | −4.104 | 2.941 | 1.855 | 1.00 | 0.00 | S |
| ATOM | 300 | H | CYS | A | 22 | −3.254 | −0.263 | 4.684 | 1.00 | 0.00 | H |
| ATOM | 301 | HA | CYS | A | 22 | −2.519 | 0.461 | 2.035 | 1.00 | 0.00 | H |
| ATOM | 302 | 1HB | CYS | A | 22 | −4.731 | 0.972 | 3.045 | 1.00 | 0.00 | H |
| ATOM | 303 | 2HB | CYS | A | 22 | −3.940 | 2.141 | 4.095 | 1.00 | 0.00 | H |
| ATOM | 304 | N | TYR | A | 23 | −0.542 | 1.906 | 2.253 | 1.00 | 0.00 | N |
| ATOM | 305 | CA | TYR | A | 23 | 0.632 | 2.766 | 2.333 | 1.00 | 0.00 | C |
| ATOM | 306 | C | TYR | A | 23 | 0.475 | 3.975 | 1.414 | 1.00 | 0.00 | C |
| ATOM | 307 | O | TYR | A | 23 | −0.119 | 3.879 | 0.340 | 1.00 | 0.00 | O |
| ATOM | 308 | CB | TYR | A | 23 | 1.891 | 1.978 | 1.958 | 1.00 | 0.00 | C |
| ATOM | 309 | CG | TYR | A | 23 | 3.143 | 2.827 | 1.888 | 1.00 | 0.00 | C |
| ATOM | 310 | CD1 | TYR | A | 23 | 3.810 | 3.214 | 3.043 | 1.00 | 0.00 | C |
| ATOM | 311 | CD2 | TYR | A | 23 | 3.656 | 3.238 | 0.664 | 1.00 | 0.00 | C |
| ATOM | 312 | CE1 | TYR | A | 23 | 4.953 | 3.988 | 2.982 | 1.00 | 0.00 | C |
| ATOM | 313 | CE2 | TYR | A | 23 | 4.799 | 4.012 | 0.595 | 1.00 | 0.00 | C |
| ATOM | 314 | CZ | TYR | A | 23 | 5.444 | 4.383 | 1.756 | 1.00 | 0.00 | C |
| ATOM | 315 | OH | TYR | A | 23 | 6.582 | 5.155 | 1.691 | 1.00 | 0.00 | O |
| ATOM | 316 | H | TYR | A | 23 | −0.695 | 1.383 | 1.439 | 1.00 | 0.00 | H |
| ATOM | 317 | HA | TYR | A | 23 | 0.721 | 3.110 | 3.352 | 1.00 | 0.00 | H |
| ATOM | 318 | 1HB | TYR | A | 23 | 2.056 | 1.207 | 2.695 | 1.00 | 0.00 | H |
| ATOM | 319 | 2HB | TYR | A | 23 | 1.745 | 1.521 | 0.990 | 1.00 | 0.00 | H |
| ATOM | 320 | HD1 | TYR | A | 23 | 3.423 | 2.901 | 4.001 | 1.00 | 0.00 | H |
| ATOM | 321 | HD2 | TYR | A | 23 | 3.150 | 2.947 | −0.244 | 1.00 | 0.00 | H |
| ATOM | 322 | HE1 | TYR | A | 23 | 5.457 | 4.279 | 3.891 | 1.00 | 0.00 | H |
| ATOM | 323 | HE2 | TYR | A | 23 | 5.184 | 4.323 | −0.367 | 1.00 | 0.00 | H |
| ATOM | 324 | HH | TYR | A | 23 | 7.126 | 4.862 | 0.957 | 1.00 | 0.00 | H |
| ATOM | 325 | N | CYS | A | 24 | 1.016 | 5.111 | 1.846 | 1.00 | 0.00 | N |
| ATOM | 326 | CA | CYS | A | 24 | 0.939 | 6.340 | 1.066 | 1.00 | 0.00 | C |
| ATOM | 327 | C | CYS | A | 24 | 2.304 | 6.702 | 0.490 | 1.00 | 0.00 | C |
| ATOM | 328 | O | CYS | A | 24 | 3.292 | 6.786 | 1.220 | 1.00 | 0.00 | O |
| ATOM | 329 | CB | CYS | A | 24 | 0.418 | 7.487 | 1.932 | 1.00 | 0.00 | C |
| ATOM | 330 | SG | CYS | A | 24 | −1.207 | 7.168 | 2.691 | 1.00 | 0.00 | S |
| ATOM | 331 | H | CYS | A | 24 | 1.476 | 5.122 | 2.711 | 1.00 | 0.00 | H |
| ATOM | 332 | HA | CYS | A | 24 | 0.249 | 6.173 | 0.251 | 1.00 | 0.00 | H |
| ATOM | 333 | 1HB | CYS | A | 24 | 1.121 | 7.673 | 2.730 | 1.00 | 0.00 | H |
| ATOM | 334 | 2HB | CYS | A | 24 | 0.329 | 8.375 | 1.325 | 1.00 | 0.00 | H |
| ATOM | 335 | N | ARG | A | 25 | 2.356 | 6.913 | −0.822 | 1.00 | 0.00 | N |
| ATOM | 336 | CA | ARG | A | 25 | 3.604 | 7.265 | −1.486 | 1.00 | 0.00 | C |
| ATOM | 337 | C | ARG | A | 25 | 4.021 | 8.698 | −1.170 | 1.00 | 0.00 | C |
| ATOM | 338 | O | ARG | A | 25 | 5.153 | 9.100 | −1.439 | 1.00 | 0.00 | O |
| ATOM | 339 | CB | ARG | A | 25 | 3.483 | 7.070 | −2.992 | 1.00 | 0.00 | C |
| ATOM | 340 | CG | ARG | A | 25 | 4.456 | 6.045 | −3.554 | 1.00 | 0.00 | C |
| ATOM | 341 | CD | ARG | A | 25 | 5.307 | 6.628 | −4.673 | 1.00 | 0.00 | C |
| ATOM | 342 | NE | ARG | A | 25 | 4.492 | 7.237 | −5.725 | 1.00 | 0.00 | N |
| ATOM | 343 | CZ | ARG | A | 25 | 4.271 | 8.546 | −5.838 | 1.00 | 0.00 | C |
| ATOM | 344 | NH1 | ARG | A | 25 | 4.785 | 9.398 | −4.957 | 1.00 | 0.00 | N |
| ATOM | 345 | NH2 | ARG | A | 25 | 3.530 | 9.007 | −6.836 | 1.00 | 0.00 | N |
| ATOM | 346 | H | ARG | A | 25 | 1.537 | 6.832 | −1.354 | 1.00 | 0.00 | H |
| ATOM | 347 | HA | ARG | A | 25 | 4.361 | 6.607 | −1.118 | 1.00 | 0.00 | H |
| ATOM | 348 | 1HB | ARG | A | 25 | 2.482 | 6.744 | −3.217 | 1.00 | 0.00 | H |
| ATOM | 349 | 2HB | ARG | A | 25 | 3.664 | 8.013 | −3.479 | 1.00 | 0.00 | H |
| ATOM | 350 | 1HG | ARG | A | 25 | 5.108 | 5.711 | −2.761 | 1.00 | 0.00 | H |
| ATOM | 351 | 2HG | ARG | A | 25 | 3.896 | 5.206 | −3.940 | 1.00 | 0.00 | H |
| ATOM | 352 | 1HD | ARG | A | 25 | 5.964 | 7.376 | −4.258 | 1.00 | 0.00 | H |
| ATOM | 353 | 2HD | ARG | A | 25 | 5.899 | 5.834 | −5.105 | 1.00 | 0.00 | H |
| ATOM | 354 | HE | ARG | A | 25 | 4.092 | 6.637 | −6.388 | 1.00 | 0.00 | H |
| ATOM | 355 | 1HH1 | ARG | A | 25 | 5.340 | 9.063 | −4.199 | 1.00 | 0.00 | H |
| ATOM | 356 | 2HH1 | ARG | A | 25 | 4.612 | 10.378 | −5.054 | 1.00 | 0.00 | H |
| ATOM | 357 | 1HH2 | ARG | A | 25 | 3.138 | 8.372 | −7.502 | 1.00 | 0.00 | H |
| ATOM | 358 | 2HH2 | ARG | A | 25 | 3.363 | 9.989 | −6.923 | 1.00 | 0.00 | H |
| ATOM | 359 | N | PHE | A | 26 | 3.103 | 9.457 | −0.593 | 1.00 | 0.00 | N |
| ATOM | 360 | CA | PHE | A | 26 | 3.368 | 10.838 | −0.231 | 1.00 | 0.00 | C |
| ATOM | 361 | C | PHE | A | 26 | 2.610 | 11.196 | 1.043 | 1.00 | 0.00 | C |
| ATOM | 362 | O | PHE | A | 26 | 2.227 | 10.315 | 1.814 | 1.00 | 0.00 | O |
| ATOM | 363 | CB | PHE | A | 26 | 2.964 | 11.769 | −1.380 | 1.00 | 0.00 | C |
| ATOM | 364 | CG | PHE | A | 26 | 4.046 | 12.730 | −1.783 | 1.00 | 0.00 | C |
| ATOM | 365 | CD1 | PHE | A | 26 | 4.284 | 13.002 | −3.120 | 1.00 | 0.00 | C |
| ATOM | 366 | CD2 | PHE | A | 26 | 4.824 | 13.363 | −0.826 | 1.00 | 0.00 | C |
| ATOM | 367 | CE1 | PHE | A | 26 | 5.279 | 13.884 | −3.496 | 1.00 | 0.00 | C |
| ATOM | 368 | CE2 | PHE | A | 26 | 5.820 | 14.246 | −1.195 | 1.00 | 0.00 | C |
| ATOM | 369 | CZ | PHE | A | 26 | 6.047 | 14.508 | −2.532 | 1.00 | 0.00 | C |
| ATOM | 370 | H | PHE | A | 26 | 2.225 | 9.077 | −0.401 | 1.00 | 0.00 | H |
| ATOM | 371 | HA | PHE | A | 26 | 4.427 | 10.938 | −0.049 | 1.00 | 0.00 | H |
| ATOM | 372 | 1HB | PHE | A | 26 | 2.711 | 11.173 | −2.243 | 1.00 | 0.00 | H |
| ATOM | 373 | 2HB | PHE | A | 26 | 2.099 | 12.346 | −1.083 | 1.00 | 0.00 | H |
| ATOM | 374 | HD1 | PHE | A | 26 | 3.684 | 12.516 | −3.875 | 1.00 | 0.00 | H |
| ATOM | 375 | HD2 | PHE | A | 26 | 4.647 | 13.158 | 0.221 | 1.00 | 0.00 | H |
| ATOM | 376 | HE1 | PHE | A | 26 | 5.457 | 14.087 | −4.542 | 1.00 | 0.00 | H |
| ATOM | 377 | HE2 | PHE | A | 26 | 6.419 | 14.732 | −0.439 | 1.00 | 0.00 | H |
| ATOM | 378 | HZ | PHE | A | 26 | 6.825 | 15.198 | −2.822 | 1.00 | 0.00 | H |
| ATOM | 379 | N | PHE | A | 27 | 2.389 | 12.484 | 1.258 | 1.00 | 0.00 | N |
| ATOM | 380 | CA | PHE | A | 27 | 1.674 | 12.953 | 2.425 | 1.00 | 0.00 | C |
| ATOM | 381 | C | PHE | A | 27 | 0.292 | 12.313 | 2.499 | 1.00 | 0.00 | C |
| ATOM | 382 | O | PHE | A | 27 | 0.004 | 11.530 | 3.404 | 1.00 | 0.00 | O |
| ATOM | 383 | CB | PHE | A | 27 | 1.552 | 14.479 | 2.388 | 1.00 | 0.00 | C |
| ATOM | 384 | CG | PHE | A | 27 | 2.188 | 15.144 | 1.197 | 1.00 | 0.00 | C |
| ATOM | 385 | CD1 | PHE | A | 27 | 3.479 | 15.642 | 1.273 | 1.00 | 0.00 | C |
| ATOM | 386 | CD2 | PHE | A | 27 | 1.495 | 15.270 | 0.004 | 1.00 | 0.00 | C |
| ATOM | 387 | CE1 | PHE | A | 27 | 4.067 | 16.253 | 0.182 | 1.00 | 0.00 | C |
| ATOM | 388 | CE2 | PHE | A | 27 | 2.077 | 15.878 | −1.092 | 1.00 | 0.00 | C |
| ATOM | 389 | CZ | PHE | A | 27 | 3.364 | 16.372 | −1.002 | 1.00 | 0.00 | C |
| ATOM | 390 | H | PHE | A | 27 | 2.704 | 13.137 | 0.616 | 1.00 | 0.00 | H |
| ATOM | 391 | HA | PHE | A | 27 | 2.240 | 12.666 | 3.299 | 1.00 | 0.00 | H |
| ATOM | 392 | 1HB | PHE | A | 27 | 0.513 | 14.742 | 2.382 | 1.00 | 0.00 | H |
| ATOM | 393 | 2HB | PHE | A | 27 | 2.015 | 14.880 | 3.269 | 1.00 | 0.00 | H |
| ATOM | 394 | HD1 | PHE | A | 27 | 4.030 | 15.550 | 2.199 | 1.00 | 0.00 | H |
| ATOM | 395 | HD2 | PHE | A | 27 | 0.489 | 14.884 | −0.068 | 1.00 | 0.00 | H |
| ATOM | 396 | HE1 | PHE | A | 27 | 5.073 | 16.637 | 0.254 | 1.00 | 0.00 | H |
| ATOM | 397 | HE2 | PHE | A | 27 | 1.527 | 15.970 | −2.015 | 1.00 | 0.00 | H |
| ATOM | 398 | HZ | PHE | A | 27 | 3.822 | 16.848 | −1.857 | 1.00 | 0.00 | H |
| ATOM | 399 | N | ASN | A | 28 | −0.555 | 12.648 | 1.532 | 1.00 | 0.00 | N |
| ATOM | 400 | CA | ASN | A | 28 | −1.907 | 12.105 | 1.474 | 1.00 | 0.00 | C |
| ATOM | 401 | C | ASN | A | 28 | −2.437 | 12.127 | 0.043 | 1.00 | 0.00 | C |
| ATOM | 402 | O | ASN | A | 28 | −3.223 | 13.002 | −0.322 | 1.00 | 0.00 | O |
| ATOM | 403 | CB | ASN | A | 28 | −2.840 | 12.901 | 2.391 | 1.00 | 0.00 | C |
| ATOM | 404 | CG | ASN | A | 28 | −2.653 | 14.399 | 2.249 | 1.00 | 0.00 | C |
| ATOM | 405 | OD1 | ASN | A | 28 | −1.863 | 15.009 | 2.970 | 1.00 | 0.00 | O |
| ATOM | 406 | ND2 | ASN | A | 28 | −3.381 | 15.001 | 1.317 | 1.00 | 0.00 | N |
| ATOM | 407 | H | ASN | A | 28 | −0.262 | 13.273 | 0.836 | 1.00 | 0.00 | H |
| ATOM | 408 | HA | ASN | A | 28 | −1.868 | 11.081 | 1.815 | 1.00 | 0.00 | H |
| ATOM | 409 | 1HB | ASN | A | 28 | −3.864 | 12.661 | 2.146 | 1.00 | 0.00 | H |
| ATOM | 410 | 2HB | ASN | A | 28 | −2.646 | 12.626 | 3.417 | 1.00 | 0.00 | H |
| ATOM | 411 | 1HD2 | ASN | A | 28 | −3.990 | 14.452 | 0.779 | 1.00 | 0.00 | H |
| ATOM | 412 | 2HD2 | ASN | A | 28 | −3.280 | 15.969 | 1.203 | 1.00 | 0.00 | H |
| ATOM | 413 | N | ALA | A | 29 | −2.002 | 11.166 | −0.766 | 1.00 | 0.00 | N |
| ATOM | 414 | CA | ALA | A | 29 | −2.439 | 11.092 | −2.155 | 1.00 | 0.00 | C |
| ATOM | 415 | C | ALA | A | 29 | −2.410 | 9.657 | −2.673 | 1.00 | 0.00 | C |
| ATOM | 416 | O | ALA | A | 29 | −3.456 | 9.057 | −2.917 | 1.00 | 0.00 | O |
| ATOM | 417 | CB | ALA | A | 29 | −1.571 | 11.985 | −3.028 | 1.00 | 0.00 | C |
| ATOM | 418 | H | ALA | A | 29 | −1.370 | 10.496 | −0.422 | 1.00 | 0.00 | H |
| ATOM | 419 | HA | ALA | A | 29 | −3.453 | 11.460 | −2.205 | 1.00 | 0.00 | H |
| ATOM | 420 | 1HB | ALA | A | 29 | −1.817 | 11.822 | −4.068 | 1.00 | 0.00 | H |
| ATOM | 421 | 2HB | ALA | A | 29 | −0.530 | 11.747 | −2.865 | 1.00 | 0.00 | H |
| ATOM | 422 | 3HB | ALA | A | 29 | −1.748 | 13.020 | −2.773 | 1.00 | 0.00 | H |
| ATOM | 423 | N | PHE | A | 30 | −1.210 | 9.116 | −2.842 | 1.00 | 0.00 | N |
| ATOM | 424 | CA | PHE | A | 30 | −1.053 | 7.753 | −3.336 | 1.00 | 0.00 | C |
| ATOM | 425 | C | PHE | A | 30 | −1.161 | 6.744 | −2.196 | 1.00 | 0.00 | C |
| ATOM | 426 | O | PHE | A | 30 | −0.212 | 6.017 | −1.903 | 1.00 | 0.00 | O |
| ATOM | 427 | CB | PHE | A | 30 | 0.293 | 7.598 | −4.048 | 1.00 | 0.00 | C |
| ATOM | 428 | CG | PHE | A | 30 | 0.239 | 7.930 | −5.513 | 1.00 | 0.00 | C |
| ATOM | 429 | CD1 | PHE | A | 30 | −0.238 | 9.157 | −5.942 | 1.00 | 0.00 | C |
| ATOM | 430 | CD2 | PHE | A | 30 | 0.667 | 7.012 | −6.459 | 1.00 | 0.00 | C |
| ATOM | 431 | CE1 | PHE | A | 30 | −0.288 | 9.465 | −7.289 | 1.00 | 0.00 | C |
| ATOM | 432 | CE2 | PHE | A | 30 | 0.621 | 7.315 | −7.807 | 1.00 | 0.00 | C |
| ATOM | 433 | CZ | PHE | A | 30 | 0.142 | 8.542 | −8.222 | 1.00 | 0.00 | C |
| ATOM | 434 | H | PHE | A | 30 | −0.411 | 9.645 | −2.633 | 1.00 | 0.00 | H |
| ATOM | 435 | HA | PHE | A | 30 | −1.848 | 7.564 | −4.042 | 1.00 | 0.00 | H |
| ATOM | 436 | 1HB | PHE | A | 30 | 1.015 | 8.254 | −3.586 | 1.00 | 0.00 | H |
| ATOM | 437 | 2HB | PHE | A | 30 | 0.627 | 6.575 | −3.950 | 1.00 | 0.00 | H |
| ATOM | 438 | HD1 | PHE | A | 30 | −0.575 | 9.880 | −5.213 | 1.00 | 0.00 | H |
| ATOM | 439 | HD2 | PHE | A | 30 | 1.041 | 6.052 | −6.136 | 1.00 | 0.00 | H |
| ATOM | 440 | HE1 | PHE | A | 30 | −0.662 | 10.426 | −7.610 | 1.00 | 0.00 | H |
| ATOM | 441 | HE2 | PHE | A | 30 | 0.956 | 6.591 | −8.535 | 1.00 | 0.00 | H |
| ATOM | 442 | HZ | PHE | A | 30 | 0.105 | 8.780 | −9.276 | 1.00 | 0.00 | H |
| ATOM | 443 | N | CYS | A | 31 | −2.327 | 6.707 | −1.557 | 1.00 | 0.00 | N |
| ATOM | 444 | CA | CYS | A | 31 | −2.563 | 5.790 | −0.448 | 1.00 | 0.00 | C |
| ATOM | 445 | C | CYS | A | 31 | −3.314 | 4.548 | −0.921 | 1.00 | 0.00 | C |
| ATOM | 446 | O | CYS | A | 31 | −4.529 | 4.582 | −1.107 | 1.00 | 0.00 | O |
| ATOM | 447 | CB | CYS | A | 31 | −3.354 | 6.487 | 0.658 | 1.00 | 0.00 | C |
| ATOM | 448 | SG | CYS | A | 31 | −2.525 | 7.950 | 1.359 | 1.00 | 0.00 | S |
| ATOM | 449 | H | CYS | A | 31 | −3.045 | 7.312 | −1.839 | 1.00 | 0.00 | H |
| ATOM | 450 | HA | CYS | A | 31 | −1.602 | 5.488 | −0.058 | 1.00 | 0.00 | H |
| ATOM | 451 | 1HB | CYS | A | 31 | −4.306 | 6.809 | 0.261 | 1.00 | 0.00 | H |
| ATOM | 452 | 2HB | CYS | A | 31 | −3.525 | 5.788 | 1.463 | 1.00 | 0.00 | H |
| ATOM | 453 | N | TYR | A | 32 | −2.581 | 3.455 | −1.109 | 1.00 | 0.00 | N |
| ATOM | 454 | CA | TYR | A | 32 | −3.182 | 2.203 | −1.560 | 1.00 | 0.00 | C |
| ATOM | 455 | C | TYR | A | 32 | −2.630 | 1.021 | −0.770 | 1.00 | 0.00 | C |
| ATOM | 456 | O | TYR | A | 32 | −1.607 | 1.136 | −0.097 | 1.00 | 0.00 | O |
| ATOM | 457 | CB | TYR | A | 32 | −2.933 | 1.994 | −3.056 | 1.00 | 0.00 | C |
| ATOM | 458 | CG | TYR | A | 32 | −1.568 | 2.454 | −3.520 | 1.00 | 0.00 | C |
| ATOM | 459 | CD1 | TYR | A | 32 | −0.413 | 2.005 | −2.892 | 1.00 | 0.00 | C |
| ATOM | 460 | CD2 | TYR | A | 32 | −1.437 | 3.336 | −4.584 | 1.00 | 0.00 | C |
| ATOM | 461 | CE1 | TYR | A | 32 | 0.837 | 2.423 | −3.314 | 1.00 | 0.00 | C |
| ATOM | 462 | CE2 | TYR | A | 32 | −0.192 | 3.757 | −5.011 | 1.00 | 0.00 | C |
| ATOM | 463 | CZ | TYR | A | 32 | 0.940 | 3.299 | −4.374 | 1.00 | 0.00 | C |
| ATOM | 464 | OH | TYR | A | 32 | 2.182 | 3.717 | −4.797 | 1.00 | 0.00 | O |
| ATOM | 465 | H | TYR | A | 32 | −1.616 | 3.491 | −0.942 | 1.00 | 0.00 | H |
| ATOM | 466 | HA | TYR | A | 32 | −4.246 | 2.268 | −1.389 | 1.00 | 0.00 | H |
| ATOM | 467 | 1HB | TYR | A | 32 | −3.022 | 0.942 | −3.285 | 1.00 | 0.00 | H |
| ATOM | 468 | 2HB | TYR | A | 32 | −3.676 | 2.542 | −3.616 | 1.00 | 0.00 | H |
| ATOM | 469 | HD1 | TYR | A | 32 | −0.497 | 1.318 | −2.062 | 1.00 | 0.00 | H |
| ATOM | 470 | HD2 | TYR | A | 32 | −2.326 | 3.694 | −5.082 | 1.00 | 0.00 | H |
| ATOM | 471 | HE1 | TYR | A | 32 | 1.723 | 2.062 | −2.814 | 1.00 | 0.00 | H |
| ATOM | 472 | HE2 | TYR | A | 32 | −0.112 | 4.445 | −5.842 | 1.00 | 0.00 | H |
| ATOM | 473 | HH | TYR | A | 32 | 2.193 | 4.675 | −4.859 | 1.00 | 0.00 | H |
| ATOM | 474 | N | CYS | A | 33 | −3.317 | −0.116 | −0.854 | 1.00 | 0.00 | N |
| ATOM | 475 | CA | CYS | A | 33 | −2.895 | −1.319 | −0.141 | 1.00 | 0.00 | C |
| ATOM | 476 | C | CYS | A | 33 | −1.444 | −1.671 | −0.460 | 1.00 | 0.00 | C |
| ATOM | 477 | O | CYS | A | 33 | −0.976 | −1.477 | −1.582 | 1.00 | 0.00 | O |
| ATOM | 478 | CB | CYS | A | 33 | −3.810 | −2.497 | −0.489 | 1.00 | 0.00 | C |
| ATOM | 479 | SG | CYS | A | 33 | −3.186 | −4.122 | 0.064 | 1.00 | 0.00 | S |
| ATOM | 480 | H | CYS | A | 33 | −4.127 | −0.145 | −1.406 | 1.00 | 0.00 | H |
| ATOM | 481 | HA | CYS | A | 33 | −2.975 | −1.118 | 0.918 | 1.00 | 0.00 | H |
| ATOM | 482 | 1HB | CYS | A | 33 | −4.773 | −2.344 | −0.029 | 1.00 | 0.00 | H |
| ATOM | 483 | 2HB | CYS | A | 33 | −3.933 | −2.542 | −1.562 | 1.00 | 0.00 | H |
| ATOM | 484 | N | ARG | A | 34 | −0.747 | −2.197 | 0.540 | 1.00 | 0.00 | N |
| ATOM | 485 | CA | ARG | A | 34 | 0.647 | −2.591 | 0.386 | 1.00 | 0.00 | C |
| ATOM | 486 | C | ARG | A | 34 | 0.907 | −3.912 | 1.103 | 1.00 | 0.00 | C |
| ATOM | 487 | O | ARG | A | 34 | 0.720 | −4.018 | 2.316 | 1.00 | 0.00 | O |
| ATOM | 488 | CB | ARG | A | 34 | 1.573 | −1.506 | 0.936 | 1.00 | 0.00 | C |
| ATOM | 489 | CG | ARG | A | 34 | 3.050 | −1.838 | 0.796 | 1.00 | 0.00 | C |
| ATOM | 490 | CD | ARG | A | 34 | 3.891 | −0.582 | 0.635 | 1.00 | 0.00 | C |
| ATOM | 491 | NE | ARG | A | 34 | 5.318 | −0.855 | 0.790 | 1.00 | 0.00 | N |
| ATOM | 492 | CZ | ARG | A | 34 | 6.078 | −1.392 | −0.162 | 1.00 | 0.00 | C |
| ATOM | 493 | NH1 | ARG | A | 34 | 5.551 | −1.715 | −1.337 | 1.00 | 0.00 | N |
| ATOM | 494 | NH2 | ARG | A | 34 | 7.366 | −1.606 | 0.062 | 1.00 | 0.00 | N |
| ATOM | 495 | H | ARG | A | 34 | −1.183 | −2.327 | 1.407 | 1.00 | 0.00 | H |
| ATOM | 496 | HA | ARG | A | 34 | 0.842 | −2.721 | −0.669 | 1.00 | 0.00 | H |
| ATOM | 497 | 1HB | ARG | A | 34 | 1.382 | −0.584 | 0.408 | 1.00 | 0.00 | H |
| ATOM | 498 | 2HB | ARG | A | 34 | 1.357 | −1.361 | 1.984 | 1.00 | 0.00 | H |
| ATOM | 499 | 1HG | ARG | A | 34 | 3.375 | −2.365 | 1.681 | 1.00 | 0.00 | H |
| ATOM | 500 | 2HG | ARG | A | 34 | 3.187 | −2.466 | −0.072 | 1.00 | 0.00 | H |
| ATOM | 501 | 1HD | ARG | A | 34 | 3.717 | −0.172 | −0.349 | 1.00 | 0.00 | H |
| ATOM | 502 | 2HD | ARG | A | 34 | 3.587 | 0.137 | 1.381 | 1.00 | 0.00 | H |
| ATOM | 503 | HE | ARG | A | 34 | 5.731 | −0.626 | 1.648 | 1.00 | 0.00 | H |
| ATOM | 504 | 1HH1 | ARG | A | 34 | 4.580 | −1.557 | −1.513 | 1.00 | 0.00 | H |
| ATOM | 505 | 2HH1 | ARG | A | 34 | 6.128 | −2.118 | −2.048 | 1.00 | 0.00 | H |
| ATOM | 506 | 1HH2 | ARG | A | 34 | 7.767 | −1.363 | 0.946 | 1.00 | 0.00 | H |
| ATOM | 507 | 2HH2 | ARG | A | 34 | 7.937 | −2.009 | −0.653 | 1.00 | 0.00 | H |
| ATOM | 508 | N | LYS | A | 35 | 1.330 | −4.919 | 0.345 | 1.00 | 0.00 | N |
| ATOM | 509 | CA | LYS | A | 35 | 1.609 | −6.237 | 0.908 | 1.00 | 0.00 | C |
| ATOM | 510 | C | LYS | A | 35 | 3.021 | −6.317 | 1.486 | 1.00 | 0.00 | C |
| ATOM | 511 | O | LYS | A | 35 | 3.507 | −7.404 | 1.797 | 1.00 | 0.00 | O |
| ATOM | 512 | CB | LYS | A | 35 | 1.423 | −7.316 | −0.160 | 1.00 | 0.00 | C |
| ATOM | 513 | CG | LYS | A | 35 | 0.015 | −7.886 | −0.206 | 1.00 | 0.00 | C |
| ATOM | 514 | CD | LYS | A | 35 | −0.268 | −8.568 | −1.535 | 1.00 | 0.00 | C |
| ATOM | 515 | CE | LYS | A | 35 | 0.516 | −9.866 | −1.674 | 1.00 | 0.00 | C |
| ATOM | 516 | NZ | LYS | A | 35 | −0.380 | −11.052 | −1.750 | 1.00 | 0.00 | N |
| ATOM | 517 | H | LYS | A | 35 | 1.454 | −4.775 | −0.617 | 1.00 | 0.00 | H |
| ATOM | 518 | HA | LYS | A | 35 | 0.899 | −6.410 | 1.703 | 1.00 | 0.00 | H |
| ATOM | 519 | 1HB | LYS | A | 35 | 1.649 | −6.893 | −1.127 | 1.00 | 0.00 | H |
| ATOM | 520 | 2HB | LYS | A | 35 | 2.110 | −8.125 | 0.039 | 1.00 | 0.00 | H |
| ATOM | 521 | 1HG | LYS | A | 35 | −0.098 | −8.608 | 0.589 | 1.00 | 0.00 | H |
| ATOM | 522 | 2HG | LYS | A | 35 | −0.694 | −7.082 | −0.067 | 1.00 | 0.00 | H |
| ATOM | 523 | 1HD | LYS | A | 35 | −1.322 | −8.790 | −1.598 | 1.00 | 0.00 | H |
| ATOM | 524 | 2HD | LYS | A | 35 | 0.012 | −7.902 | −2.338 | 1.00 | 0.00 | H |
| ATOM | 525 | 1HE | LYS | A | 35 | 1.109 | −9.817 | −2.576 | 1.00 | 0.00 | H |
| ATOM | 526 | 2HE | LYS | A | 35 | 1.169 | −9.973 | −0.820 | 1.00 | 0.00 | H |
| ATOM | 527 | 1HZ | LYS | A | 35 | −0.019 | −11.814 | −1.140 | 1.00 | 0.00 | H |
| ATOM | 528 | 2HZ | LYS | A | 35 | −0.430 | −11.401 | −2.728 | 1.00 | 0.00 | H |
| ATOM | 529 | 3HZ | LYS | A | 35 | −1.340 | −10.797 | −1.436 | 1.00 | 0.00 | H |
| ATOM | 530 | N | LEU | A | 36 | 3.679 | −5.167 | 1.633 | 1.00 | 0.00 | N |
| ATOM | 531 | CA | LEU | A | 36 | 5.025 | −5.128 | 2.175 | 1.00 | 0.00 | C |
| ATOM | 532 | C | LEU | A | 36 | 5.990 | −5.915 | 1.305 | 1.00 | 0.00 | C |
| ATOM | 533 | O | LEU | A | 36 | 5.918 | −7.141 | 1.223 | 1.00 | 0.00 | O |
| ATOM | 534 | CB | LEU | A | 36 | 5.039 | −5.664 | 3.604 | 1.00 | 0.00 | C |
| ATOM | 535 | CG | LEU | A | 36 | 4.725 | −4.611 | 4.663 | 1.00 | 0.00 | C |
| ATOM | 536 | CD1 | LEU | A | 36 | 3.342 | −4.840 | 5.256 | 1.00 | 0.00 | C |
| ATOM | 537 | CD2 | LEU | A | 36 | 5.783 | −4.613 | 5.756 | 1.00 | 0.00 | C |
| ATOM | 538 | H | LEU | A | 36 | 3.251 | −4.329 | 1.374 | 1.00 | 0.00 | H |
| ATOM | 539 | HA | LEU | A | 36 | 5.339 | −4.096 | 2.190 | 1.00 | 0.00 | H |
| ATOM | 540 | 1HB | LEU | A | 36 | 4.308 | −6.458 | 3.678 | 1.00 | 0.00 | H |
| ATOM | 541 | 2HB | LEU | A | 36 | 6.017 | −6.072 | 3.807 | 1.00 | 0.00 | H |
| ATOM | 542 | HG | LEU | A | 36 | 4.730 | −3.640 | 4.190 | 1.00 | 0.00 | H |
| ATOM | 543 | 1HD1 | LEU | A | 36 | 2.594 | −4.694 | 4.490 | 1.00 | 0.00 | H |
| ATOM | 544 | 2HD1 | LEU | A | 36 | 3.176 | −4.138 | 6.060 | 1.00 | 0.00 | H |
| ATOM | 545 | 3HD1 | LEU | A | 36 | 3.276 | −5.848 | 5.638 | 1.00 | 0.00 | H |
| ATOM | 546 | 1HD2 | LEU | A | 36 | 5.340 | −4.294 | 6.687 | 1.00 | 0.00 | H |
| ATOM | 547 | 2HD2 | LEU | A | 36 | 6.581 | −3.934 | 5.486 | 1.00 | 0.00 | H |
| ATOM | 548 | 3HD2 | LEU | A | 36 | 6.183 | −5.609 | 5.870 | 1.00 | 0.00 | H |
| ATOM | 549 | N | GLY | A | 37 | 6.890 | −5.193 | 0.658 | 1.00 | 0.00 | N |
| ATOM | 550 | CA | GLY | A | 37 | 7.871 | −5.823 | −0.206 | 1.00 | 0.00 | C |
| ATOM | 551 | C | GLY | A | 37 | 9.003 | −6.472 | 0.567 | 1.00 | 0.00 | C |
| ATOM | 552 | O | GLY | A | 37 | 10.174 | −6.191 | 0.315 | 1.00 | 0.00 | 0 |
| ATOM | 553 | H | GLY | A | 37 | 6.888 | −4.219 | 0.770 | 1.00 | 0.00 | H |
| ATOM | 554 | 1HA | GLY | A | 37 | 7.376 | −6.578 | −0.800 | 1.00 | 0.00 | H |
| ATOM | 555 | 2HA | GLY | A | 37 | 8.284 | −5.076 | −0.868 | 1.00 | 0.00 | H |
| ATOM | 556 | N | THR | A | 38 | 8.654 | −7.347 | 1.506 | 1.00 | 0.00 | N |
| ATOM | 557 | CA | THR | A | 38 | 9.649 | −8.042 | 2.312 | 1.00 | 0.00 | C |
| ATOM | 558 | C | THR | A | 38 | 10.046 | −9.357 | 1.655 | 1.00 | 0.00 | C |
| ATOM | 559 | O | THR | A | 38 | 9.603 | −10.429 | 2.070 | 1.00 | 0.00 | O |
| ATOM | 560 | CB | THR | A | 38 | 9.110 | −8.298 | 3.720 | 1.00 | 0.00 | C |
| ATOM | 561 | OG1 | THR | A | 38 | 7.895 | −9.024 | 3.669 | 1.00 | 0.00 | O |
| ATOM | 562 | CG2 | THR | A | 38 | 8.854 | −7.029 | 4.505 | 1.00 | 0.00 | C |
| ATOM | 563 | H | THR | A | 38 | 7.707 | −7.534 | 1.658 | 1.00 | 0.00 | H |
| ATOM | 564 | HA | THR | A | 38 | 10.522 | −7.408 | 2.379 | 1.00 | 0.00 | H |
| ATOM | 565 | HB | THR | A | 38 | 9.832 | −8.885 | 4.269 | 1.00 | 0.00 | H |
| ATOM | 566 | HG1 | THR | A | 38 | 7.196 | −8.459 | 3.332 | 1.00 | 0.00 | H |
| ATOM | 567 | 1HG2 | THR | A | 38 | 8.717 | −6.205 | 3.820 | 1.00 | 0.00 | H |
| ATOM | 568 | 2HG2 | THR | A | 38 | 9.698 | −6.827 | 5.147 | 1.00 | 0.00 | H |
| ATOM | 569 | 3HG2 | THR | A | 38 | 7.965 | −7.150 | 5.106 | 1.00 | 0.00 | H |
| ATOM | 570 | N | ALA | A | 39 | 10.878 | −9.267 | 0.625 | 1.00 | 0.00 | N |
| ATOM | 571 | CA | ALA | A | 39 | 11.335 | −10.449 | −0.099 | 1.00 | 0.00 | C |
| ATOM | 572 | C | ALA | A | 39 | 12.107 | −11.402 | 0.810 | 1.00 | 0.00 | C |
| ATOM | 573 | O | ALA | A | 39 | 12.308 | −12.568 | 0.469 | 1.00 | 0.00 | O |
| ATOM | 574 | CB | ALA | A | 39 | 12.191 | −10.040 | −1.288 | 1.00 | 0.00 | C |
| ATOM | 575 | H | ALA | A | 39 | 11.190 | −8.381 | 0.342 | 1.00 | 0.00 | H |
| ATOM | 576 | HA | ALA | A | 39 | 10.464 | −10.959 | −0.474 | 1.00 | 0.00 | H |
| ATOM | 577 | 1HB | ALA | A | 39 | 11.591 | −9.475 | −1.985 | 1.00 | 0.00 | H |
| ATOM | 578 | 2HB | ALA | A | 39 | 12.577 | −10.922 | −1.775 | 1.00 | 0.00 | H |
| ATOM | 579 | 3HB | ALA | A | 39 | 13.014 | −9.429 | −0.946 | 1.00 | 0.00 | H |
| ATOM | 580 | N | MET | A | 40 | 12.536 | −10.904 | 1.963 | 1.00 | 0.00 | N |
| ATOM | 581 | CA | MET | A | 40 | 13.283 | −11.718 | 2.914 | 1.00 | 0.00 | C |
| ATOM | 582 | C | MET | A | 40 | 12.334 | −12.525 | 3.796 | 1.00 | 0.00 | C |
| ATOM | 583 | O | MET | A | 40 | 12.618 | −13.671 | 4.143 | 1.00 | 0.00 | O |
| ATOM | 584 | CB | MET | A | 40 | 14.178 | −10.833 | 3.783 | 1.00 | 0.00 | C |
| ATOM | 585 | CG | MET | A | 40 | 15.537 | −11.448 | 4.081 | 1.00 | 0.00 | C |
| ATOM | 586 | SD | MET | A | 40 | 16.877 | −10.242 | 4.016 | 1.00 | 0.00 | S |
| ATOM | 587 | CE | MET | A | 40 | 16.815 | −9.759 | 2.292 | 1.00 | 0.00 | C |
| ATOM | 588 | H | MET | A | 40 | 12.347 | −9.970 | 2.179 | 1.00 | 0.00 | H |
| ATOM | 589 | HA | MET | A | 40 | 13.902 | −12.400 | 2.352 | 1.00 | 0.00 | H |
| ATOM | 590 | 1HB | MET | A | 40 | 14.336 | −9.892 | 3.277 | 1.00 | 0.00 | H |
| ATOM | 591 | 2HB | MET | A | 40 | 13.679 | −10.645 | 4.722 | 1.00 | 0.00 | H |
| ATOM | 592 | 1HG | MET | A | 40 | 15.512 | −11.882 | 5.069 | 1.00 | 0.00 | H |
| ATOM | 593 | 2HG | MET | A | 40 | 15.733 | −12.222 | 3.353 | 1.00 | 0.00 | H |
| ATOM | 594 | 1HE | MET | A | 40 | 16.197 | −8.880 | 2.186 | 1.00 | 0.00 | H |
| ATOM | 595 | 2HE | MET | A | 40 | 16.398 | −10.566 | 1.708 | 1.00 | 0.00 | H |
| ATOM | 596 | 3HE | MET | A | 40 | 17.814 | −9.541 | 1.943 | 1.00 | 0.00 | H |
| ATOM | 597 | N | ASN | A | 41 | 11.206 | −11.920 | 4.149 | 1.00 | 0.00 | N |
| ATOM | 598 | CA | ASN | A | 41 | 10.211 | −12.581 | 4.986 | 1.00 | 0.00 | C |
| ATOM | 599 | C | ASN | A | 41 | 8.806 | −12.072 | 4.664 | 1.00 | 0.00 | C |
| ATOM | 600 | O | ASN | A | 41 | 8.174 | −11.402 | 5.481 | 1.00 | 0.00 | O |
| ATOM | 601 | CB | ASN | A | 41 | 10.529 | −12.356 | 6.467 | 1.00 | 0.00 | C |
| ATOM | 602 | CG | ASN | A | 41 | 11.147 | −13.578 | 7.117 | 1.00 | 0.00 | C |
| ATOM | 603 | OD1 | ASN | A | 41 | 11.058 | −14.688 | 6.591 | 1.00 | 0.00 | O |
| ATOM | 604 | ND2 | ASN | A | 41 | 11.780 | −13.382 | 8.268 | 1.00 | 0.00 | N |
| ATOM | 605 | H | ASN | A | 41 | 11.035 | −11.007 | 3.837 | 1.00 | 0.00 | H |
| ATOM | 606 | HA | ASN | A | 41 | 10.253 | −13.639 | 4.775 | 1.00 | 0.00 | H |
| ATOM | 607 | 1HB | ASN | A | 41 | 11.223 | −11.534 | 6.559 | 1.00 | 0.00 | H |
| ATOM | 608 | 2HB | ASN | A | 41 | 9.618 | −12.114 | 6.994 | 1.00 | 0.00 | H |
| ATOM | 609 | 1HD2 | ASN | A | 41 | 11.812 | −12.471 | 8.629 | 1.00 | 0.00 | H |
| ATOM | 610 | 2HD2 | ASN | A | 41 | 12.189 | −14.156 | 8.710 | 1.00 | 0.00 | H |
| ATOM | 611 | N | PRO | A | 42 | 8.299 | −12.384 | 3.459 | 1.00 | 0.00 | N |
| ATOM | 612 | CA | PRO | A | 42 | 6.966 | −11.956 | 3.028 | 1.00 | 0.00 | C |
| ATOM | 613 | C | PRO | A | 42 | 5.855 | −12.784 | 3.665 | 1.00 | 0.00 | C |
| ATOM | 614 | O | PRO | A | 42 | 6.027 | −13.974 | 3.927 | 1.00 | 0.00 | O |
| ATOM | 615 | CB | PRO | A | 42 | 7.002 | −12.182 | 1.518 | 1.00 | 0.00 | C |
| ATOM | 616 | CG | PRO | A | 42 | 7.954 | −13.313 | 1.332 | 1.00 | 0.00 | C |
| ATOM | 617 | CD | PRO | A | 42 | 8.987 | −13.177 | 2.420 | 1.00 | 0.00 | C |
| ATOM | 618 | HA | PRO | A | 42 | 6.800 | −10.909 | 3.235 | 1.00 | 0.00 | H |
| ATOM | 619 | 1HB | PRO | A | 42 | 6.013 | −12.435 | 1.165 | 1.00 | 0.00 | H |
| ATOM | 620 | 2HB | PRO | A | 42 | 7.351 | −11.289 | 1.023 | 1.00 | 0.00 | H |
| ATOM | 621 | 1HG | PRO | A | 42 | 7.431 | −14.252 | 1.428 | 1.00 | 0.00 | H |
| ATOM | 622 | 2HG | PRO | A | 42 | 8.422 | −13.243 | 0.362 | 1.00 | 0.00 | H |
| ATOM | 623 | 1HD | PRO | A | 42 | 9.265 | −14.149 | 2.800 | 1.00 | 0.00 | H |
| ATOM | 624 | 2HD | PRO | A | 42 | 9.857 | −12.655 | 2.051 | 1.00 | 0.00 | H |
| ATOM | 625 | N | CYS | A | 43 | 4.715 | −12.147 | 3.909 | 1.00 | 0.00 | N |
| ATOM | 626 | CA | CYS | A | 43 | 3.582 | −12.799 | 4.505 | 1.00 | 0.00 | C |
| ATOM | 627 | C | CYS | A | 43 | 2.788 | −13.563 | 3.458 | 1.00 | 0.00 | C |
| ATOM | 628 | O | CYS | A | 43 | 3.287 | −14.505 | 2.841 | 1.00 | 0.00 | O |
| ATOM | 629 | CB | CYS | A | 43 | 2.717 | −11.741 | 5.173 | 1.00 | 0.00 | C |
| ATOM | 630 | SG | CYS | A | 43 | 3.620 | −10.655 | 6.325 | 1.00 | 0.00 | S |
| ATOM | 631 | H | CYS | A | 43 | 4.627 | −11.204 | 3.684 | 1.00 | 0.00 | H |
| ATOM | 632 | HA | CYS | A | 43 | 3.923 | −13.485 | 5.246 | 1.00 | 0.00 | H |
| ATOM | 633 | 1HB | CYS | A | 43 | 2.274 | −11.119 | 4.409 | 1.00 | 0.00 | H |
| ATOM | 634 | 2HB | CYS | A | 43 | 1.942 | −12.227 | 5.718 | 1.00 | 0.00 | H |
| ATOM | 635 | N | SER | A | 44 | 1.554 | −13.151 | 3.270 | 1.00 | 0.00 | N |
| ATOM | 636 | CA | SER | A | 44 | 0.670 | −13.781 | 2.308 | 1.00 | 0.00 | C |
| ATOM | 637 | C | SER | A | 44 | 0.936 | −13.263 | 0.900 | 1.00 | 0.00 | C |
| ATOM | 638 | O | SER | A | 44 | 0.285 | −12.326 | 0.439 | 1.00 | 0.00 | O |
| ATOM | 639 | CB | SER | A | 44 | −0.791 | −13.539 | 2.688 | 1.00 | 0.00 | C |
| ATOM | 640 | OG | SER | A | 44 | −1.145 | −14.269 | 3.851 | 1.00 | 0.00 | O |
| ATOM | 641 | H | SER | A | 44 | 1.231 | −12.404 | 3.801 | 1.00 | 0.00 | H |
| ATOM | 642 | HA | SER | A | 44 | 0.869 | −14.835 | 2.337 | 1.00 | 0.00 | H |
| ATOM | 643 | 1HB | SER | A | 44 | −0.943 | −12.488 | 2.879 | 1.00 | 0.00 | H |
| ATOM | 644 | 2HB | SER | A | 44 | −1.429 | −13.852 | 1.874 | 1.00 | 0.00 | H |
| ATOM | 645 | HG | SER | A | 44 | −1.908 | −14.821 | 3.663 | 1.00 | 0.00 | H |
| ATOM | 646 | N | ARG | A | 45 | 1.898 | −13.880 | 0.219 | 1.00 | 0.00 | N |
| ATOM | 647 | CA | ARG | A | 45 | 2.250 | −13.481 | −1.138 | 1.00 | 0.00 | C |
| ATOM | 648 | C | ARG | A | 45 | 2.882 | −14.639 | −1.904 | 1.00 | 0.00 | C |
| ATOM | 649 | O | ARG | A | 45 | 3.804 | −14.443 | −2.697 | 1.00 | 0.00 | O |
| ATOM | 650 | CB | ARG | A | 45 | 3.211 | −12.291 | −1.108 | 1.00 | 0.00 | C |
| ATOM | 651 | CG | ARG | A | 45 | 3.124 | −11.405 | −2.341 | 1.00 | 0.00 | C |
| ATOM | 652 | CD | ARG | A | 45 | 4.407 | −11.447 | −3.153 | 1.00 | 0.00 | C |
| ATOM | 653 | NE | ARG | A | 45 | 5.470 | −10.655 | −2.540 | 1.00 | 0.00 | N |
| ATOM | 654 | CZ | ARG | A | 45 | 6.607 | −10.337 | −3.156 | 1.00 | 0.00 | C |
| ATOM | 655 | NH1 | ARG | A | 45 | 6.833 | −10.743 | −4.399 | 1.00 | 0.00 | N |
| ATOM | 656 | NH2 | ARG | A | 45 | 7.520 | −9.611 | −2.526 | 1.00 | 0.00 | N |
| ATOM | 657 | H | ARG | A | 45 | 2.382 | −14.621 | 0.639 | 1.00 | 0.00 | H |
| ATOM | 658 | HA | ARG | A | 45 | 1.343 | −13.184 | −1.643 | 1.00 | 0.00 | H |
| ATOM | 659 | 1HB | ARG | A | 45 | 2.988 | −11.685 | −0.241 | 1.00 | 0.00 | H |
| ATOM | 660 | 2HB | ARG | A | 45 | 4.222 | −12.661 | −1.027 | 1.00 | 0.00 | H |
| ATOM | 661 | 1HG | ARG | A | 45 | 2.307 | −11.746 | −2.960 | 1.00 | 0.00 | H |
| ATOM | 662 | 2HG | ARG | A | 45 | 2.939 | −10.387 | −2.028 | 1.00 | 0.00 | H |
| ATOM | 663 | 1HD | ARG | A | 45 | 4.737 | −12.473 | −3.230 | 1.00 | 0.00 | H |
| ATOM | 664 | 2HD | ARG | A | 45 | 4.206 | −11.061 | −4.142 | 1.00 | 0.00 | H |
| ATOM | 665 | HE | ARG | A | 45 | 5.330 | −10.342 | −1.622 | 1.00 | 0.00 | H |
| ATOM | 666 | 1HH1 | ARG | A | 45 | 6.148 | −11.291 | −4.880 | 1.00 | 0.00 | H |
| ATOM | 667 | 2HH1 | ARG | A | 45 | 7.688 | −10.501 | −4.856 | 1.00 | 0.00 | H |
| ATOM | 668 | 1HH2 | ARG | A | 45 | 7.356 | −9.303 | −1.590 | 1.00 | 0.00 | H |
| ATOM | 669 | 2HH2 | ARG | A | 45 | 8.375 | −9.373 | −2.989 | 1.00 | 0.00 | H |
| ATOM | 670 | N | THR | A | 46 | 2.382 | −15.846 | −1.661 | 1.00 | 0.00 | N |
| ATOM | 671 | CA | THR | A | 46 | 2.899 | −17.036 | −2.328 | 1.00 | 0.00 | C |
| ATOM | 672 | C | THR | A | 46 | 2.368 | −17.133 | −3.754 | 1.00 | 0.00 | C |
| ATOM | 673 | O | THR | A | 46 | 2.909 | −17.946 | −4.533 | 1.00 | 0.00 | O |
| ATOM | 674 | CB | THR | A | 46 | 2.522 | −18.293 | −1.543 | 1.00 | 0.00 | C |
| ATOM | 675 | OG1 | THR | A | 46 | 2.802 | −18.127 | −0.165 | 1.00 | 0.00 | O |
| ATOM | 676 | CG2 | THR | A | 46 | 3.250 | −19.534 | −2.014 | 1.00 | 0.00 | C |
| ATOM | 677 | OXT | THR | A | 46 | 1.413 | −16.397 | −4.081 | 1.00 | 0.00 | O |
| ATOM | 678 | H | THR | A | 46 | 1.648 | −15.940 | −1.019 | 1.00 | 0.00 | H |
| ATOM | 679 | HA | THR | A | 46 | 3.976 | −16.954 | −2.363 | 1.00 | 0.00 | H |
| ATOM | 680 | HB | THR | A | 46 | 1.461 | −18.470 | −1.656 | 1.00 | 0.00 | H |
| ATOM | 681 | HG1 | THR | A | 46 | 2.011 | −17.825 | 0.289 | 1.00 | 0.00 | H |
| ATOM | 682 | 1HG2 | THR | A | 46 | 3.933 | −19.271 | −2.809 | 1.00 | 0.00 | H |
| ATOM | 683 | 2HG2 | THR | A | 46 | 2.534 | −20.255 | −2.378 | 1.00 | 0.00 | H |
| ATOM | 684 | 3HG2 | THR | A | 46 | 3.803 | −19.960 | −1.191 | 1.00 | 0.00 | H |
| TER | 685 | | THR | A | 46 | | | | | | |

### Example 3

### Screening of Mini-AGGRPs for Melanocortin Receptor Activity

The mini-AGRP, given by the sequence: Ac-CVRLHESCLGQQVPCCDPAATCYCRFFNAFCYCR-NH₂ (SEQ ID NO:5) was synthesized using Fmoc solid phase chemistry (where Ac is an acetyl group and —NH₂ denotes a C-terminal amide). The peptide was prepared as a single chain and then partially purified by HPLC. The peptide was then folded under oxidizing conditions to form the disulfide bonds, and then repurified. HPLC indicated a uniform product and mass spectrometry demonstrated loss of 8.0 AMUs consistent with formation of four disulfide bonds. The folding yield was greater than 80% as determined by HPLC.

Amino acid analysis verified the correct omposition and allowed for the preparation of stock solutions of known mini-AGRP concentration (to within 5%).

The mini-AGRP binding was tested by examining its ability to displace NDP-MSH, a well established high affinity MCR agonist. To within experimental error, the mini-AGRP displaced NDP-MSH at each of the melanocortin receptors (MC IR, MC3R, MC4R and MC5R) with potency equivalent to that of AGRP(87-132) (MARP). Antagonist activity was then determined using a cAMP assay. With both brain receptors MC3R and MC4R, mini-AGRP exhibited potent activity as determined by its ability to suppress α-MSH stimulated cAMP production. Taken together, these studies demonstrate that this mini-AGRP is readily synthesized, folded to a uniform product, and exhibits MCR binding, selectivity and antagonist activity comparable to that of the AGRP C-terminal domain (AGRP(87-132); MARP).

### Example 4

### Synthesis and Assay of Melanocortin Receptor Binding Ligands (Peptidomimetics)

A) Synthesis of Non-peptide Compounds

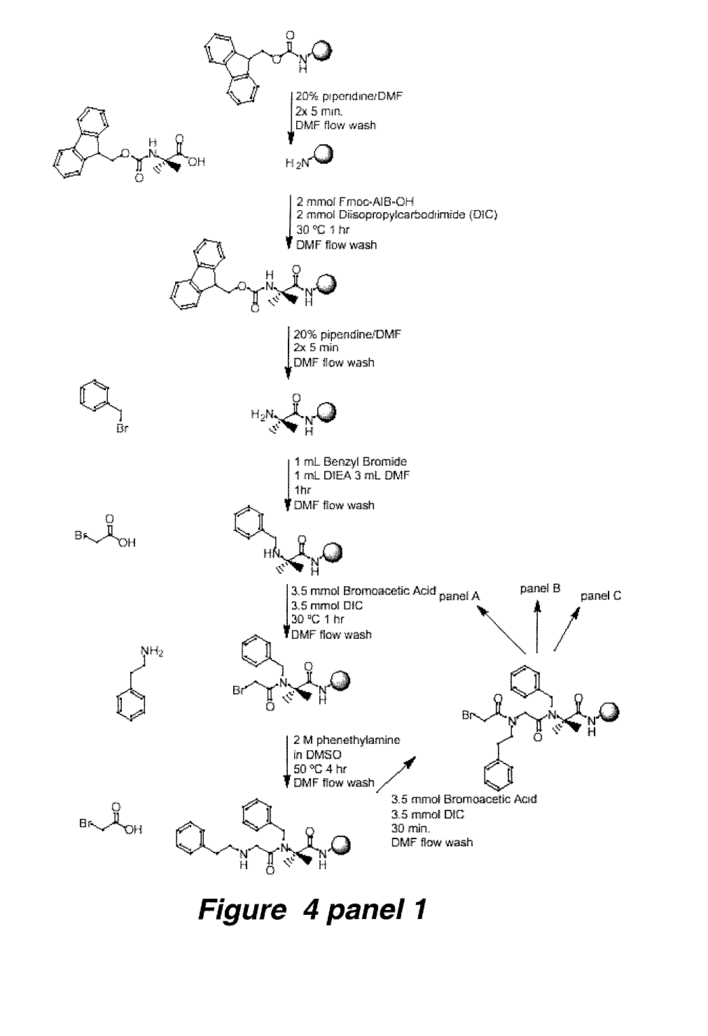

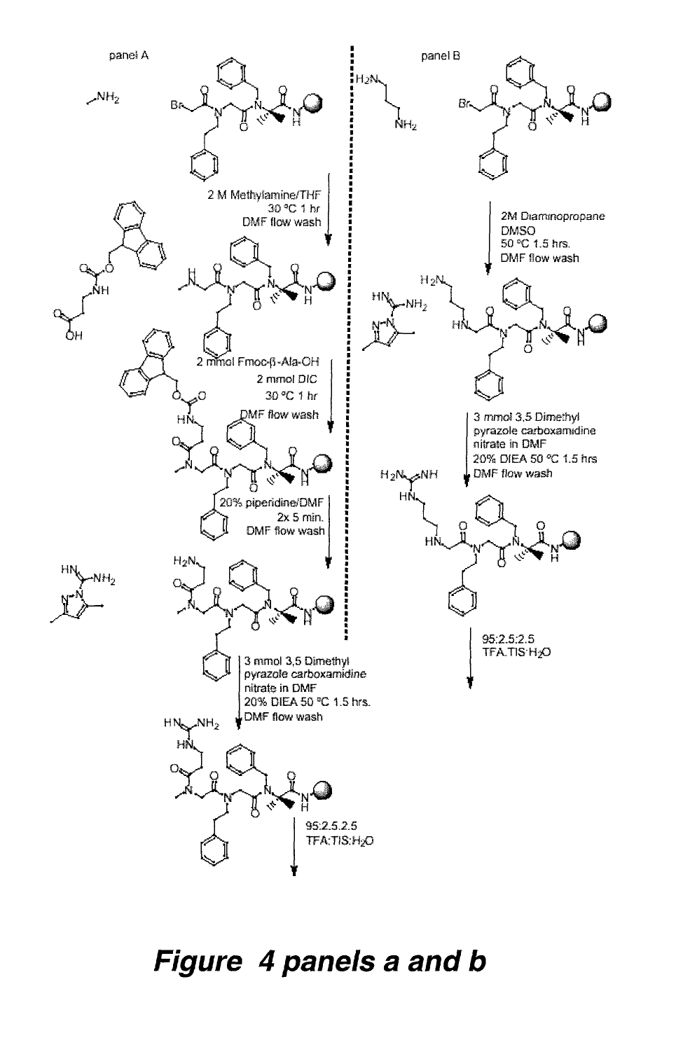

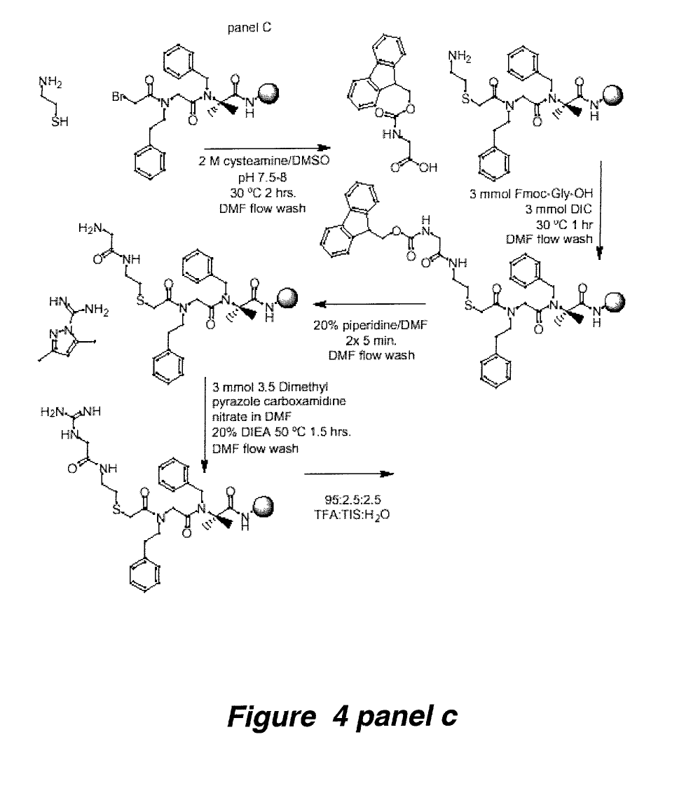

A synthesis scheme for the peptidomimetics is illustrated in FIG. 4. To 20% Piperidine/DMF 2×5 min treated and DMF washed Rink Amide-MBHA resin (Novabiochem, Switzerland), 5 equivalents of Fmoc-Aminoisobutyric acid (Fmoc-Aib) or 7 of Bromoacetic acid (Aldrich, USA) in DMF, activated as the symmetric anhydride by incubating 30 min. with Diisopropylcarbodiimide (DIC), is reacted for 20 min., then washed with DMF. The washed resin, in the case of Fmoc-Aib treated with 20% Piperidine/DMF 2×5 min. and DMF washed, is reacted with 20-40 equivalents, concentration 2-4 M, of primary or secondary amine or bromide of choice in DMSO or DMF with the appropriate tertiary amine (note: methylamine is only available as a 2M solution in various solvents). This is known as the submonomer approach (Zuckermann et al., J Amer Chem Soc 1992).

The process is repeated 1-2 times, until yielding a complete molecule, or intermediate. To the intermediate, 7 equivalents of bromoacetic acid or 5-8 equivalents of a carboxylic acid of choice, activated as the symmetric anhydride by incubating 30 min. with Diisopropylcarbodiimide (DIC) or as the assymetric anhydride, in the case of an enantiomeric carboxylic acid, is reacted for 0.5-2 hr., then washed with DMF. If the intermidiate is brominated then reacted with 20 equivalents of the thiol or 2-4 M of the primary or secondary amine of choice, if the thiol or amine contains one or more accessible amine then the carboxylic acid of choice is coupled or the amine is guanylated. If the intermediate has an accessible amine then one of the following may happen: 1) alkylation by the primary or secondary bromide of choice 2) guanylation 3) a carboxylic acid of choice is coupled. The reaction of amines with 3,5-Dimethylpyrazole-1-carboxamidine nitrate or O-methylisourea is well known in the art. The resulting non-peptides are cleaved, usually, but not limited to, by exposing the resin bound molecules to 95:5 TFA:H₂O. The cleavage from Resin is done in 95:2.5:2.5 TFA:TIS:water.

B) Activity Assay

HEK 293 cells stably expressing the human melanocortin receptors (hMCR's) 1-5 are grown in media. After removal from media, cells are washed twice with MEM (Life Technologies, Gaithersberg, Md.) and then preincubated with AGRP (87-132) in 0.5 ml MEM 0.2% BSA for 30 min. before incubation with ¹²⁵I-NDP-MSH or 125 I-AGRP (87-132). Concentrations of cold ligand are added from 100 mM -1 nM. Binding reactions are terminated by washing cells twice with MEM 0.2% BSA. The cells are lysed with 0.1 N NaOH 1% Triton X, and radioactivity of the lysate is quantified with a gamma-analytical counter. AGRP (87-132) hMC4R IC₅₀=9 nM±1.7 nM. IC₅₀<100 mM is considered a specific binder

It is apparent from the above results and discussion that the subject invention provides an important new class of MCR ligands, where the ligands have broad ranging applicability as either MCR antagonists and agonists and therefore find use in a variety of therapeutic and related applications.

It is understood that the examples and embodiments described herein are for illustrative purposes only and that various modifications or changes in light thereof will be suggested to persons skilled in the art and are to be included within the purview of this application and scope of the appended claims. All publications, patents, and patent applications cited herein are hereby incorporated by reference in their entirety for all purposes.

What is claimed is:

1. A method of modulating the activity of a melanocortin receptor, said method comprising contacting said receptor with a peptide having the formula:
CX¹X²X³X⁴X⁵X⁶CX⁷X⁸X⁹X¹⁰X¹¹X¹²CCDPX¹³ATCYCX¹⁴X¹⁵X¹⁶NAFCYCRₙ
wherein
X¹, X², X³, X⁴, X⁵, X⁶, X⁷, X⁸, X⁹, X¹⁰, X¹¹, X¹², X¹³, X¹⁴, X¹⁵, and X¹⁶ are independently selected amino acids, and
n is zero or one.

2. The method of claim 1, wherein X¹, X², X³, X⁴, X^{5, X}⁶, X⁷, X⁸, X⁹, X¹⁰, X¹¹, X¹², X¹³, X¹⁴, X¹⁵, and X¹⁶ are independently selected from the group consisting of alanine, asparagine, arginine,aspartic acid, cysteine, glutamine, glutamic acid, glycine, histidine, isoleucine, leucine, lysine, methionine, phenylalanine, proline, serine, threonine, tryptophan, tyrosine, and valine.

3. The method of claim 1, wherein said peptide is not CVRLHESCLGQQVPCCDPAATCYCRFFNAFCYC (SEQ ID NO:3).

4. The method of claim 1, wherein X¹X²X³X⁴X⁵X⁶ is VRLHES or conservative substitutions thereof.

5. The method of claim 4, wherein X¹X²X³X⁴X⁵X⁶ is VRLHES.

6. The method of claim 1, wherein X⁷X⁸X⁹X¹⁰X¹¹X¹² is LGQQVP or conservative substitutions thereof.

7. The method of claim 6, wherein X⁷X⁸X⁹X¹⁰X¹¹X¹² is LGQQVP.

8. The method of claim 7, wherein X⁷X⁸X⁹X¹⁰X¹¹X¹² is LGQQVP or conservative substitutions thereof.

9. The method of claim 8, wherein X⁷X⁸X⁹X¹⁰X¹¹X¹² is LGQQVP.

10. The method of claim 1, wherein X¹³ is not a cysteine.

11. The method of claim 1, wherein X¹³ is A.

12. The method of claim 1, wherein X¹⁴X¹⁵X¹⁶ is RFF or conservative substitutions thereof.

13. The method of claim 4, wherein X¹⁴X¹⁵X¹⁶ is RFF or conservative substitutions thereof.

14. The method of claim 8, wherein X¹⁴X¹⁵X¹⁶ is RFF or conservative substitutions thereof.

15. The method of claim 1, wherein said receptor is in a cell culture.

16. The method of claim 1, wherein said receptor is in vivo culture.

17. The method of claim 1, wherein said receptor is an MC3 receptor.

18. The method of claim 1, wherein said receptor is an MC4 receptor.

19. A library for screening for modulators of a melanocortin receptor, said library comprising a plurality of polypeptide members wherein said members have the formula:
CX¹X²X³X⁴X⁵X⁶CX⁷X⁸X⁹X¹⁰X¹¹X¹²CCDPX¹³ATCYCX¹⁴X¹⁵X¹⁶NAFCYCRₙ
wherein
X¹, X², X³, X⁴, X⁵, X⁶, X⁷, X⁸, X⁹, X¹⁰, X¹¹, X¹², X¹³, X¹⁴, X¹⁵, and X¹⁶ are independently selected amino acids, and
n is zero or one.

20. The library of claim 19, wherein X¹, X², X³, X⁴, X⁵, X⁶, X⁷, X⁸, X⁹, X¹⁰, X¹¹, X¹², X¹³, X¹⁴, X¹⁵, and X¹⁶ are independently selected from the group consisting of aspartic acid, alanine, asparagine, arginine,cysteine, glutamine, glutamic acid, glycine, histidine, isoleucine, leucine, lysine, methionine, phenylalanine, proline, serine, threonine, tryptophan, tyrosine, and valine.

21. The method of claim 19, wherein X¹X²X³X⁴X⁵X⁶ is VRLHES or conservative substitutions thereof.

22. The method of claim 21, wherein X¹X²X³X⁴X⁵X⁶ is VRLHES.

23. The method of claim 19, wherein X⁷X⁸X⁹X¹⁰X¹¹X¹² is LGQQVP or conservative substitutions thereof.

24. The method of claim 23, wherein X⁷X⁸X⁹X¹⁰X¹¹X¹² is LGQQVP.

25. The method of claim 24, wherein X⁷X⁸X⁹X¹⁰X¹¹X¹² is LGQQVP or conservative substitutions thereof.

26. The method of claim 25, wherein X⁷X⁸X⁹X¹⁰X¹¹X¹² is LGQQVP.

27. The method of claim 19, wherein X¹³ is not a cysteine.

28. The method of claim 19, wherein X¹³ is A.

29. The method of claim 19, wherein X¹⁴X¹⁵X¹⁶ is RFF or conservative substitutions thereof.

30. The method of claim 21, wherein X¹⁴X¹⁵X¹⁶ is RFF or conservative substitutions thereof.

31. The method of claim 25, wherein X¹⁴X¹⁵X¹⁶ is RFF or conservative substitutions thereof.

32. A method of prescreening for a modulator of a melanocortin receptor, said method comprising:
i) contacting a melanocortin receptor a peptide having the formula:
CX¹X²X³X⁴X⁵X⁶CX⁷X⁸X⁹X¹⁰X¹¹X¹²CCDPX¹³ATCYCX¹⁴X¹⁵X¹⁶NAFCYCRₙ
wherein
X¹, X², X³, X⁴, X⁵, X⁶, X⁷, X⁸, X⁹, X¹⁰, X¹¹, X¹², X¹³, X¹⁴, X¹⁵, and X¹⁶ are independently selected amino acids, and
n is zero or one; and
ii) detecting binding of said peptide to said melanocortin receptor wherein specific binding of said peptide to said melanocortin receptor indicates that said peptide is a potential modulator of said melanocortin receptor.

33. The method of claim 32, wherein said peptide is not CVRLHESCLGQQVPCCDPAATCYCRFFNAFCYC (SEQ ID NO:3).

34. The method of claim 32, wherein said melanocortin receptor is selected from the group consisting of MC3r, and MC4r.

35. A method of screening for a modulator of melanocortin receptor activity, said method comprising:
i) contacting a melanocortin receptor with a peptide having the formula:
CX¹X²X³X⁴X⁵X⁶CX⁷X⁸X⁹X¹⁰X¹¹X¹²CCDPX¹³ATCYCX¹⁴X¹⁵X¹⁶NAFCYCRₙ
wherein
X¹, X², X³, X⁴, X⁵, X⁶, X⁷, X⁸, X⁹, X¹⁰, X¹¹, X¹², X¹³, X¹⁴, X¹⁵, and X¹⁶ are independently selected amino acids, and
n is zero or one; and
ii) detecting activity of said melanocortin receptor wherein a difference in activity of said receptor, as compared to a control, indicates that said peptide is a modulator of melanocortin receptor activity.

36. The method of claim 35, wherein said control is a negative control comprising the same assay without said peptide.

37. The method of claim 35, wherein said peptide is not CVRLHESCLGQQVPCCDPAATCYCRFFNAFCYC (SEQ ID NO:3).

38. The method of claim 35, wherein said melanocortin receptor is selected from the group consisting of MC3r, and MC4r.

39. A polypeptide comprising a peptide sequence having the formula 35 wherein said polypeptide is not AGRP and said polypeptide is not MARP.

40. The polypeptide of claim 39, wherein said polypeptide excludes one or more of the final 13 residues of MARP (residues 34-46 of MARP).

41. The polypeptide of claim 39, wherein said polyypeptide has the formula:
CX¹X²X³X⁴X⁵X⁶CX⁷X⁸X⁹X¹⁰X¹¹X¹²CCDPX¹³ATCYCX¹⁴X¹⁵X¹⁶NAFCYCRₙ
wherein
X¹, X², X³, X⁴, X⁵, X⁶, X⁷, X⁸, X⁹, X¹⁰, X¹¹, X¹², X¹³, X¹⁴, X¹⁵, and X¹⁶ are independently selected amino acids, and
n is zero or one.

42. The polypeptide of claim 41, wherein said polypeptide is not CVRLHESCLGQQVPCCDPAATCYCRFFNAFCYC (SEQ ID NO:3).

43. A pharmaceutical composition comprising:
a pharmaceutically acceptable excipient; and
a polypeptide having the formula:
CX¹X²X³X⁴X⁵X⁶CX⁷X⁸X⁹X¹⁰X¹¹X¹²CCDPX¹³ATCYCX¹⁴X¹⁵X¹⁶NAFCYCRₙ
wherein
X¹, X², X³, X⁴, X⁵, X⁶, X⁷, X⁸, X⁹, X¹⁰, X¹¹, X¹², X¹³, X¹⁴, X¹⁵, and X¹⁶ are independently selected amino acids, and
n is zero or one.

44. The composition of claim 43, wherein said polypeptide is not CVRLHESCLGQQVPCCDPAATCYCRFFNAFCYC (SEQ ID NO:3).

45. A method of identifying a compound that modulates ligand binding to a melanocortin receptor, said method comprising: modeling test compounds that fit spatially into a melanocortin receptor ligand binding site of interest using an atomic structural model of a melanocortin receptor binding region or portion thereof; screening said test compounds in an assay characterized by binding of a test compound to a melanocortin receptor ligand binding site; and identifying a test compound that modulates ligand binding to said melanocortin receptor.

46. The method of claim 45, wherein said melanocortin receptor binding region comprises the minimized agouti related protein receptor binding region or portion thereof.

47. The method of claim 45, wherein said atomic structural model comprises atomic coordinates of amino acid residues corresponding to residues 1-18 of the N-terminal loop of the minimized agouti related protein (residues 1-18 of SEQ ID NO:2), residues 19-13 of the central loop of the minimized agouti related protein (residues 19-34 of SEQ ID NO:2), and residues 35-46 of the C-terminal loop of the minimized agouti related protein (residues 35-46 of SEQ ID NO:2).

48. The method of claim 45, wherein said atomic structural model comprises atomic coordinates of amino acid residues corresponding to residues 19-34 of the central loop of the minimized agouti related protein (residues 19-34 of SEQ ID NO:2) and at least residues 15-18 of the N-terminal loop of the minimized agouti related protein (residues 15-18 of SEQ ID NO:2).

49. The method of claim 45, wherein said atomic structural model comprises atomic coordinates of amino acid residues corresponding to residues 19-34 of the central loop of the minimized agouti related protein (residues 19-34 of SEQ ID NO:2) and at least 20% of the contiguous or non-contiguous residues or their atoms are selected from residues 1-18 of the N-terminal loop of the minimized agouti related protein (residues 1-18 of SEQ ID NO:2).

50. The method of claim 45, wherein said atomic structural model comprises atomic coordinates of amino acid residues corresponding to residues 24-31 of the active loop of the minimized agouti related protein (residues 24-31 of SEQ ID NO:2).

51. The method of claim 45, wherein said atomic structural model comprises atomic coordinates of amino acid residues corresponding to residues 25-27 of the active loop of the minimized agouti related protein (residues 25-27 of SEQ ID NO:2).

52. The method of claim 45, wherein said screening is in vitro.

53. The method of claim 52, wherein said screening is high throughput screening.

54. The method of claim 45, wherein said assay is a biological assay.

55. The method of claim 45, wherein said test compound is from a library of compounds.

56. The method of claim 45, wherein said test compound is an agonist or antagonist of ligand binding.

57. The method of claim 56, wherein said test compound is a small organic molecule, a peptide, or peptidomimetic.

58. A method for identifying an agonist or antagonist of ligand binding to a melanocortin receptor, said method comprising the steps of: providing the atomic coordinates of a melanocortin receptor binding region or portion thereof to a computerized modeling system; modeling compounds which match or mimic the receptor binding region and thus fit spatially into the melanocortin receptor ligand binding site; and identifying in an assay for melanocortin receptor activity a compound that increases or decreases the activity of said melanocortin receptor by binding the ligand binding site of said melanocortin receptor, whereby an agonist or antagonist of ligand binding is identified.

59. The method of claim 58, wherein said melanocortin receptor binding region comprises the minimized agouti related protein receptor binding region or portion thereof.

60. A machine-readable data storage medium, comprising a data storage material encoded with machine readable data which, when using a machine programmed with instructions for using said data, is capable of displaying a graphical three-dimensional representation of a molecule that binds a melanocortin receptor comprising structure coordinates of amino acid residues corresponding to residues 1-18 of the N-terminal loop of the minimized agouti related protein (residues 1-18 of SEQ ID NO:2), residues 19-13 of the central loop of the minimized agouti related protein (residues 19-34 of SEQ ID NO:2), and residues 35-46 of the C-terminal loop of the minimized agouti related protein (residues 35-46 of SEQ ID NO:2), or a homologue of said molecule.

61. The machine readable storage medium of claim 60, wherein said molecule is a melanocortin receptor agonist.

62. The machine readable storage medium of claim 60, wherein said molecule is a melanocortin receptor antagonist.

63. The machine-readable data storage medium according to claim 60 herein said molecule is defined by the set of structure coordinates depicted in Table 4 or Table 5, or a homologue of said molecule, said homologue having a root mean square deviation from the backbone atoms of said amino acids of not more than 2.54 Å.

64. A machine-readable data storage medium comprising a data storage material encoded with a first set of machine readable data which, when combined with a second set of machine readable data, using a machine programmed with instructions for using said first set of data and said second set of data, can determine at least a portion of the structure coordinates corresponding to the second set of machine readable data, wherein: said first set of data comprises a Fourier transform of at least a portion of the structural coordinates selected from the group consisting of coordinates depicted in Table 4 or Table 5; and said second set of data comprises an X-ray diffraction pattern of a molecule.

65. An NMR structure of the minimized agouti related protein, embodied in a computer readable media.

66. A polypeptide comprising the amino acid sequence:
CVRLHESCLGQQVPCCDPAATCYCRFFNAFCYC (SEQ ID NO:3)
or a modified form thereof, wherein said polypeptide is not a full-length AGRP and said polypeptide is not a MARP.

67. The polypeptide of claim 66, wherein the polypeptide is chemically synthesized.

68. A method of treating a disease state in mammals that is alleviated by treatment with a polypeptide having an amino acid sequence:
CVRLHESCLGQQVPCCDPAATCYCRFFNAFCYC (SEQ ID NO:3)
which method comprises administering to a mammal in need of such a treatment a therapeutically effective amount of said polypeptide, or a pharmaceutically acceptable salt thereof.

69. The method of claim 68, wherein said disease state is a wasting syndrome.

70. A pharmaceutical composition comprising a therapeutically effective amount of a polypeptide of the sequence:
CVRLHESCLGQQVPCCDPAATCYCRFFNAFCYC (SEQ ID NO:3)
or a pharmaceutically acceptable salt thereof.

71. A non-peptide melanocortin receptor ligand of the structural formula: 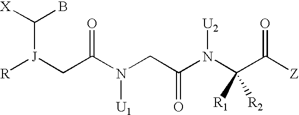
wherein
B, U₁, U₂, R, R₁, and R₂ are independently selected from the group consisting of: hydrogen, alkyl, derivatized alkyl, cycloalkyl, derivatized cycloalkyl, halocycloalkyl, aloxycycloalkyl, heterocycloalkyl, aryl, arylalkyl, heteroaryl, and heteroarylalkyl;
J is selected from the group consisting of carbon, nitrogen, silicon, and sulfur;
X is selected from the group consisting of hydrogen, carbon, nitrogen, oxygen, silicon, and sulfur; and
Z is selected from the group consisting of a continuing peptide bond, a hydroxyl; —NH₂—, —NH-(n), and —N-(n,n′), and —O-(y), where where n and n′ are independently selected from the group consisting of alkyl, alkenyl, alkynyl, cycloalkyl, aryl, arylalkyl, heteroaryl, heteroarylalkyl, and a derivatized form thereof, and y is selected from the group consisting of alkyl, alkenyl, alkynyl, cycloalkyl, aryl, arylalkyl, heteroaryl heteroarylalkyl, and a derivatized form thereof.

72. The non-peptide melanocortin receptor ligand according to claim 71, wherein said ligand is a ligand for a melanocortin receptor selected from the group consisting of MC3r and MC4r.

73. The non-peptide melanocortin receptor ligand according to claim 71, wherein said ligand has a molecular weight ranging from about 200 to 1000 daltons.

74. The non-peptide melanocortin receptor ligand according to claim 71, wherein said ligand has a structure that mimics the backbone of the AGRP active loop.

75. The non-peptide melanocortin receptor ligand according to claim 71, wherein said ligand comprises a terminal gaunidino moiety.

76. The non-peptide melanocortin receptor ligand according to claim 71, wherein said ligand comprises at least one methylbenzyl moiety.

77. The non-peptide melanocortin receptor ligand according to claim 71, wherein said ligand has the structural formula:

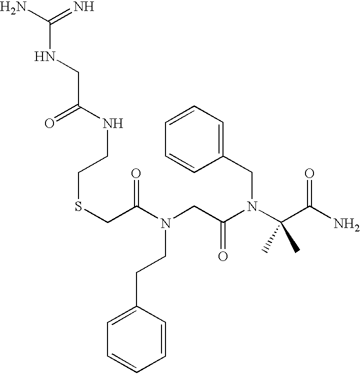

78. The non-peptide melanocortin receptor ligand according to claim 71, wherein said ligand has the structural formula:

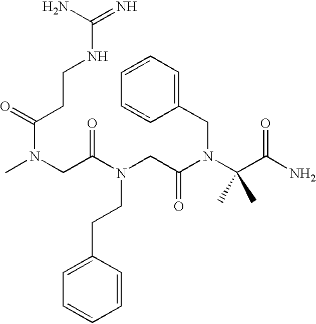

79. The non-peptide melanocortin receptor ligand according to claim 71, wherein said ligand has the structural formula:

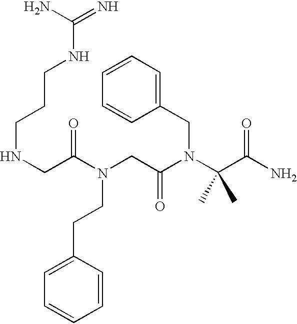

80. The non-peptide melanocortin receptor ligand according to claim 71, wherein said ligand has the structural formula:

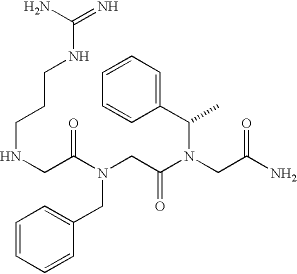

81. A pharmaceutical preparation of a non-peptide melanocortin receptor ligand according to claim 71.

82. The pharmaceutical preparation according to claim 81, wherein said ligand is a melanocortin receptor antagonist.

83. The pharmaceutical preparation according to claim 82, wherein said ligand is a melanocortin receptor agonist.

84. A method for modulating a melanocortin receptor mediated physiological process, said method comprising:
contacting said melanocortin receptor with a non-peptide melanocortin receptor ligand according to claim 71.

85. The method according to claim 84, wherein said ligand is a melanocortin receptor agonist.

86. The method according to claim 84, wherein said ligand is a melanocortin receptor antagonist.
